SEQUENCE LISTING (176 sequences)

SEQ ID NO: 1 - protein, 87 aa, Listeria monocytogenes
MNINDLIREIKNKDYTVKLSGTDSNSITQLIIRVNNDGNEYVISESENESIVEKFISAFKNGWNQEYEDEEEFYNDMQTITLKSELN

SEQ ID NO: 2 - DNA, 261 bp, Listeria monocytogenes
atgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaac

SEQ ID NO: 3 - protein, 7 aa, Simian virus 40
PKKKRKV

SEQ ID NO: 4 - DNA, 21 bp, Simian virus 40
ccaaagaagaagcggaaggtc

SEQ ID NO: 5 - protein, 1367 aa, Artificial
  Cas9
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - DNA, 4101 bp, Artificial
  Cas9 encoding
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 7 - protein, 8 aa, Listeria monocytogenes
  MISC_FEATURE (5)..(6): site of receptor domain insertion
GWNQEYED

SEQ ID NO: 8 - protein, 10 aa, Artificial
  fusion site corresponding to amino acids 62 to 69 of ACRIIA4, variant #2
  MISC_FEATURE (6)..(7): site of receptor domain insertion
GWNQEGGYED

SEQ ID NO: 9 - protein, 12 aa, Artificial
  fusion site corresponding to amino acids 62 to 69 of ACRIIA4, variant #3
  MISC_FEATURE (7)..(8): site of receptor domain insertion
GWNQESGGSYED

SEQ ID NO: 10 - protein, 7 aa, Artificial
  fusion site corresponding to amino acids 62 to 69 of ACRIIA4, variant #4
  MISC_FEATURE (4)..(5): site of receptor domain insertion
GWNQYED

SEQ ID NO: 11 - protein, 9 aa, Artificial
  fusion site corresponding to amino acids 62 to 69 of ACRIIA4, variant #5
  MISC_FEATURE (5)..(6): site of receptor domain insertion
GWNQGGYED

SEQ ID NO: 12 - protein, 11 aa, Artificial
  fusion site corresponding to amino acids 62 to 69 of ACRIIA4, variant #6
  MISC_FEATURE (6)..(7): site of receptor domain insertion
GWNQSGGSYED

SEQ ID NO: 13 - protein, 6 aa, Artificial
  fusion site corresponding to amino acids 62 to 69 of ACRIIA4, variant #7
  MISC_FEATURE (3)..(4): site of receptor domain insertion
GWNYED

SEQ ID NO: 14 - protein, 8 aa, Artificial
  fusion site corresponding to amino acids 62 to 69 of ACRIIA4, variant #8
  MISC_FEATURE (4)..(5): site of receptor domain insertion
GWNGGYED

SEQ ID NO: 15 - protein, 10 aa, Artificial
  fusion site corresponding to amino acids 62 to 69 of ACRIIA4, variant #9
  MISC_FEATURE (5)..(6): site of receptor domain insertion
GWNSGGSYED

SEQ ID NO: 16 - protein, 12 aa, Artificial
  fusion site corresponding to amino acids 62 to 69 of ACRIIA4, variant #10
  MISC_FEATURE (6)..(7): site of receptor domain insertion
GWNSGGGGSYED

SEQ ID NO: 17 - protein, 12 aa, Artificial
  fusion site corresponding to amino acids 62 to 69 of ACRIIA4, variant #11
  MISC_FEATURE (6)..(7): site of receptor domain insertion
GWNGSGGSGYED

SEQ ID NO: 18 - protein, 20 aa, Artificial
  fusion site corresponding to amino acids 62 to 69 of ACRIIA4, variant #12
  MISC_FEATURE (10)..(11): site of receptor domain insertion
GWNGGSGGSGGSGGSGGYED

SEQ ID NO: 19 - protein, 21 aa, Artificial
  fusion site corresponding to amino acids 62 to 69 of ACRIIA4, variant #13
  MISC_FEATURE (11)..(12): site of receptor domain insertion
GWNQGGSGGSGGSGGSGGYED

SEQ ID NO: 20 - protein, 10 aa, Artificial
  fusion site corresponding to amino acids 62 to 69 of ACRIIA4, variant #Rap4
  MISC_FEATURE (5)..(6): site of receptor domain insertion
GWNSGGSYED

SEQ ID NO: 21 - protein, 11 aa, Artificial
  fusion site corresponding to amino acids 62 to 69 of ACRIIA4, variant #Rap5
  MISC_FEATURE (6)..(7): site of receptor domain insertion
GWNQSGGSYED

SEQ ID NO: 22 - protein, 14 aa, Artificial
  fusion site corresponding to amino acids 62 to 69 of ACRIIA4, variant #Rap6
  MISC_FEATURE (8)..(9): site of receptor domain insertion
GWNQEGSGGSGYED

SEQ ID NO: 23 - protein, 22 aa, Artificial
  N-terminal fusion including amino acids 2 to 13 of ACRIIA4, variant #8
  HELIX (1)..(9): LOV2
  PEPTIDE (10)..(22): AcrIIA4 aa2 to 14
NIDEAAKELNINDLIREIKNKD

SEQ ID NO: 24 - protein, 22 aa, Artificial
  N-terminal fusion including amino acids 2 to 13 of ACRIIA4, variant #41
  HELIX (1)..(9): LOV2
  PEPTIDE (10)..(22): AcrIIA4 aa2 to 14
NIDELAKALNINDLIREIKNKD

SEQ ID NO: 25 - protein, 22 aa, Artificial
  N-terminal fusion including amino acids 2 to 13 of ACRIIA4, variant #51
  HELIX (1)..(9): LOV2
  HELIX (10)..(22): AcrIIA4 aa2 to 14
NIEELLKRLNINDLIREIKNKD

SEQ ID NO: 26 - protein, 20 aa, Artificial
  N-terminal fusion including amino acids 4 to 13 of ACRIIA4, variant #53-41
  HELIX (1)..(9): LOV2
  PEPTIDE (10)..(20)
NIDELAKALNDLIREIKNKD

SEQ ID NO: 27 - protein, 23 aa, Artificial
  N-terminal fusion including amino acids 2 to 13 of ACRIIA4, variant #69-41
  HELIX (1)..(9): LOV2
  MISC_FEATURE (10)..(10): Linker
  PEPTIDE (11)..(23): AcrIIA4 aa2 to 14
NIDELAKALANINDLIREIKNKD

SEQ ID NO: 28 - protein, 24 aa, Artificial
  N-terminal fusion including amino acids 2 to 13 of ACRIIA4, variant #71-51
  HELIX (1)..(9): LOV2
  MISC_FEATURE (10)..(11): Linker
  PEPTIDE (12)..(24): AcrIIA4 aa2 to 14
NIEELLKRLGPNINDLIREIKNKD

SEQ ID NO: 29 - DNA, 21 bp, Artificial
  CFTR guideRNA
gaatggtgccaggcataatcc

SEQ ID NO: 30 - DNA, 21 bp, Artificial
  CFTR_fw primer
gcacatagaacagcactcgac

SEQ ID NO: 31 - DNA, 23 bp, Artificial
  CFTR_re primer
gatccattcacagtagcttaccc

SEQ ID NO: 32 - protein, 7 aa, Artificial
  GGSGGSG linker
GGSGGSG

SEQ ID NO: 33 - protein, 7 aa, Artificial
  GSGGSGG linker
GSGGSGG

SEQ ID NO: 34 - protein, 143 aa, Avena sativa
LATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKEL

SEQ ID NO: 35 - DNA, 429 bp, Avena sativa
cttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttg

SEQ ID NO: 36 - protein, 198 aa, Artificial
  UniRapB domain
TCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLE

SEQ ID NO: 37 - DNA, 594 bp, Artificial
  UniRapB domain encoding sequence
acctgcgtggtgcattacacgggaatgctcgaggacggcaaaaagttcgattctagtcgcgacagaaataagccttttaagtttatgcttggaaagcaagaagtcatccgcgggtgggaggaaggcgttgctcaaatgtctgtaggtcaacgagctaaacttaccatttctccagattacgcatacggtgcgacggggcacggatctggctcaggatctggtgtaaaggatctgctccaagcctgggatctttattatcatgtgtttcgccgaatcagtggccctccgggcccaggaagcggactgtggcatgaaatgtggcacgagggtcttgaggaggcgagtagactgtatttcggtgagagaaatgttaaggggatgttcgaggtgctggagccgctgcacgccatgatggaaaggggcccacaaacgcttaaagaaaccagcttcaatcaggcatacggcagggatttgatggaggctcaggaatggtgtcgaaaatacatgaaatcaggtagctcaggtgggagcgggagtgggataattcctccccacgcgactctcgtgttcgacgtagaattgcttaaactggaa

SEQ ID NO: 38 - DNA, 738 bp, Artificial
  AsLOV2 AcrIIA4 insertion between G62/W63, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtggaaccaggagtacgaagatgaggaagagttctacaatgatatgcaaactatcacactgaaatctgagctgaactag

SEQ ID NO: 39 - DNA, 738 bp, Artificial
  AsLOV2 AcrIIA4 insertion between W63/N64, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgaaccaggagtacgaagatgaggaagagttctacaatgatatgcaaactatcacactgaaatctgagctgaactag

SEQ ID NO: 40 - DNA, 738 bp, Artificial
  AsLOV2 AcrIIA4 insertion between N64/Q65, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgcaggagtacgaagatgaggaagagttctacaatgatatgcaaactatcacactgaaatctgagctgaactag

SEQ ID NO: 41 - DNA, 738 bp, Artificial
  AsLOV2 AcrIIA4 insertion between Q65/E66, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccagcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttggagtacgaagatgaggaagagttctacaatgatatgcaaactatcacactgaaatctgagctgaactag

SEQ ID NO: 42 - DNA, 738 bp, Artificial
  AsLOV2 AcrIIA4 insertion between E66/Y67, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 43 - DNA, 738 bp, Artificial
  AsLOV2 AcrIIA4 insertion between Y67/E68, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtaccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttggaagatgaggaagagttctacaatgatatgcaaactatcacactgaaatctgagctgaactag

SEQ ID NO: 44 - DNA, 738 bp, Artificial
  AsLOV2 AcrIIA4 insertion between E68/D69, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaacttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttggatgaggaagagttctacaatgatatgcaaactatcacactgaaatctgagctgaactag

SEQ ID NO: 45 - DNA, 738 bp, Artificial
  AsLOV2 AcrIIA4 insertion between E85/L86, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgctgaactag

SEQ ID NO: 46 - DNA, 738 bp, Artificial
  AsLOV2 AcrIIA4 insertion between L86/N87, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgaactag

SEQ ID NO: 47 - DNA, 738 bp, Artificial
  AsLOV2 AcrIIA4 insertion after N87, encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaaccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtag

SEQ ID NO: 48 - DNA, 738 bp, Artificial; identical to SEQ ID NO: 42
  GWNQE[LOV2]YED - (#1), encoding

SEQ ID NO: 49 - DNA, 744 bp, Artificial
  GWNQEG[LOV2]GYED - (#2), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggctacgaagatgaggaagagttctacaatgatatgcaaactatcacactgaaatctgagctgaactag

SEQ ID NO: 50 - DNA, 750 bp, Artificial
  GWNQESG[LOV2]GSYED - (#3), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagctacgaagatgaggaagagttctacaatgatatgcaaactatcacactgaaatctgagctgaactag

SEQ ID NO: 51 - DNA, 735 bp, Artificial
  GWNQ[LOV2]YED - (#4), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccagcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 52 - DNA, 741 bp, Artificial
  GWNQG[LOV2]GYED - (#5), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccagggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggctacgaagatgaggaagagttctacaatgatatgcaaactatcacactgaaatctgagctgaactag

SEQ ID NO: 53 - DNA, 747 bp, Artificial
  GWNQSG[LOV2]GSYED - (#6), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccagagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagctacgaagatgaggaagagttctacaatgatatgcaaactatcacactgaaatctgagctgaactag

SEQ ID NO: 54 - DNA, 732 bp, Artificial
  GWN[LOV2]YED  - (#7), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 55 - DNA, 738 bp, Artificial
  GWNG[LOV2]GYED - (#8), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggctacgaagatgaggaagagttctacaatgatatgcaaactatcacactgaaatctgagctgaactag

SEQ ID NO: 56 - DNA, 744 bp, Artificial
  GWNSG[LOV2]GSYED - (#9), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagctacgaagatgaggaagagttctacaatgatatgcaaactatcacactgaaatctgagctgaactag

SEQ ID NO: 57 - DNA, 750 bp, Artificial
  GWNSGG[LOV2]GGSYED - (#10), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacagcggcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcggcagctacgaagatgaggaagagttctacaatgatatgcaaactatcacactgaaatctgagctgaactag

SEQ ID NO: 58 - DNA, 750 bp, Artificial
  GWNGSG[LOV2]GSGYED - (#11), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggctacgaagatgaggaagagttctacaatgatatgcaaactatcacactgaaatctgagctgaactag

SEQ ID NO: 59 - DNA, 774 bp, Artificial
  GWNGGSGGSG[LOV2]GSGGSGGYED - (#12), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacggcggcagcggcggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggcagcggcggctacgaagatgaggaagagttctacaatgatatgcaaactatcacactgaaatctgagctgaactag

SEQ ID NO: 60 - DNA, 777 bp, Artificial
  GWNQGGSGGSG[LOV2]GSGGSGGYED - (#13), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccagggcggcagcggcggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggcagcggcggctacgaagatgaggaagagttctacaatgatatgcaaactatcacactgaaatctgagctgaactag

SEQ ID NO: 61 - DNA, 903 bp, Artificial
  GWNQE[UniRap]YED - (#Rap1), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagacctgcgtggtgcattacacgggaatgctcgaggacggcaaaaagttcgattctagtcgcgacagaaataagccttttaagtttatgcttggaaagcaagaagtcatccgcgggtgggaggaaggcgttgctcaaatgtctgtaggtcaacgagctaaacttaccatttctccagattacgcatacggtgcgacggggcacggatctggctcaggatctggtgtaaaggatctgctccaagcctgggatctttattatcatgtgtttcgccgaatcagtggccctccgggcccaggaagcggactgtggcatgaaatgtggcacgagggtcttgaggaggcgagtagactgtatttcggtgagagaaatgttaaggggatgttcgaggtgctggagccgctgcacgccatgatggaaaggggcccacaaacgcttaaagaaaccagcttcaatcaggcatacggcagggatttgatggaggctcaggaatggtgtcgaaaatacatgaaatcaggtagctcaggtgggagcgggagtgggataattcctccccacgcgactctcgtgttcgacgtagaattgcttaaactggaatacgaagatgaggaagagttctacaatgatatgcaaactatcacactgaaatctgagctgaactag

SEQ ID NO: 62 - DNA, 900 bp, Artificial
  GWNQ[UniRap]YED - (#Rap2), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccagacctgcgtggtgcattacacgggaatgctcgaggacggcaaaaagttcgattctagtcgcgacagaaataagccttttaagtttatgcttggaaagcaagaagtcatccgcgggtgggaggaaggcgttgctcaaatgtctgtaggtcaacgagctaaacttaccatttctccagattacgcatacggtgcgacggggcacggatctggctcaggatctggtgtaaaggatctgctccaagcctgggatctttattatcatgtgtttcgccgaatcagtggccctccgggcccaggaagcggactgtggcatgaaatgtggcacgagggtcttgaggaggcgagtagactgtatttcggtgagagaaatgttaaggggatgttcgaggtgctggagccgctgcacgccatgatggaaaggggcccacaaacgcttaaagaaaccagcttcaatcaggcatacggcagggatttgatggaggctcaggaatggtgtcgaaaatacatgaaatcaggtagctcaggtgggagcgggagtgggataattcctccccacgcgactctcgtgttcgacgtagaattgcttaaactggaatacgaagatgaggaagagttctacaatgatatgcaaactatcacactgaaatctgagctgaactag

SEQ ID NO: 63 - DNA, 897 bp, Artificial
  GWN[UniRap]YED - (#Rap3), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacacctgcgtggtgcattacacgggaatgctcgaggacggcaaaaagttcgattctagtcgcgacagaaataagccttttaagtttatgcttggaaagcaagaagtcatccgcgggtgggaggaaggcgttgctcaaatgtctgtaggtcaacgagctaaacttaccatttctccagattacgcatacggtgcgacggggcacggatctggctcaggatctggtgtaaaggatctgctccaagcctgggatctttattatcatgtgtttcgccgaatcagtggccctccgggcccaggaagcggactgtggcatgaaatgtggcacgagggtcttgaggaggcgagtagactgtatttcggtgagagaaatgttaaggggatgttcgaggtgctggagccgctgcacgccatgatggaaaggggcccacaaacgcttaaagaaaccagcttcaatcaggcatacggcagggatttgatggaggctcaggaatggtgtcgaaaatacatgaaatcaggtagctcaggtgggagcgggagtgggataattcctccccacgcgactctcgtgttcgacgtagaattgcttaaactggaatacgaagatgaggaagagttctacaatgatatgcaaactatcacactgaaatctgagctgaactag

SEQ ID NO: 64 - DNA, 909 bp, Artificial
  GWNSG[UniRap]GSYED - (#Rap4), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacagcggcacctgcgtggtgcattacacgggaatgctcgaggacggcaaaaagttcgattctagtcgcgacagaaataagccttttaagtttatgcttggaaagcaagaagtcatccgcgggtgggaggaaggcgttgctcaaatgtctgtaggtcaacgagctaaacttaccatttctccagattacgcatacggtgcgacggggcacggatctggctcaggatctggtgtaaaggatctgctccaagcctgggatctttattatcatgtgtttcgccgaatcagtggccctccgggcccaggaagcggactgtggcatgaaatgtggcacgagggtcttgaggaggcgagtagactgtatttcggtgagagaaatgttaaggggatgttcgaggtgctggagccgctgcacgccatgatggaaaggggcccacaaacgcttaaagaaaccagcttcaatcaggcatacggcagggatttgatggaggctcaggaatggtgtcgaaaatacatgaaatcaggtagctcaggtgggagcgggagtgggataattcctccccacgcgactctcgtgttcgacgtagaattgcttaaactggaaggcagctacgaagatgaggaagagttctacaatgatatgcaaactatcacactgaaatctgagctgaactag

SEQ ID NO: 65 - DNA, 912 bp, Artificial
  GWNQSG[UniRap]GSYED - (#Rap5), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccagagcggcacctgcgtggtgcattacacgggaatgctcgaggacggcaaaaagttcgattctagtcgcgacagaaataagccttttaagtttatgcttggaaagcaagaagtcatccgcgggtgggaggaaggcgttgctcaaatgtctgtaggtcaacgagctaaacttaccatttctccagattacgcatacggtgcgacggggcacggatctggctcaggatctggtgtaaaggatctgctccaagcctgggatctttattatcatgtgtttcgccgaatcagtggccctccgggcccaggaagcggactgtggcatgaaatgtggcacgagggtcttgaggaggcgagtagactgtatttcggtgagagaaatgttaaggggatgttcgaggtgctggagccgctgcacgccatgatggaaaggggcccacaaacgcttaaagaaaccagcttcaatcaggcatacggcagggatttgatggaggctcaggaatggtgtcgaaaatacatgaaatcaggtagctcaggtgggagcgggagtgggataattcctccccacgcgactctcgtgttcgacgtagaattgcttaaactggaaggcagctacgaagatgaggaagagttctacaatgatatgcaaactatcacactgaaatctgagctgaactag

SEQ ID NO: 66 - DNA, 921 bp, Artificial
  GWNQEGSG[UniRap]GSGYED - (#Rap6), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagggcagcggcacctgcgtggtgcattacacgggaatgctcgaggacggcaaaaagttcgattctagtcgcgacagaaataagccttttaagtttatgcttggaaagcaagaagtcatccgcgggtgggaggaaggcgttgctcaaatgtctgtaggtcaacgagctaaacttaccatttctccagattacgcatacggtgcgacggggcacggatctggctcaggatctggtgtaaaggatctgctccaagcctgggatctttattatcatgtgtttcgccgaatcagtggccctccgggcccaggaagcggactgtggcatgaaatgtggcacgagggtcttgaggaggcgagtagactgtatttcggtgagagaaatgttaaggggatgttcgaggtgctggagccgctgcacgccatgatggaaaggggcccacaaacgcttaaagaaaccagcttcaatcaggcatacggcagggatttgatggaggctcaggaatggtgtcgaaaatacatgaaatcaggtagctcaggtgggagcgggagtgggataattcctccccacgcgactctcgtgttcgacgtagaattgcttaaactggaaggcagcggctacgaagatgaggaagagttctacaatgatatgcaaactatcacactgaaatctgagctgaactag

SEQ ID NO: 67 - DNA, 915 bp, Artificial
  GWNGSG[UniRap]GSGYED - (#Rap7), encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacggcagcggcacctgcgtggtgcattacacgggaatgctcgaggacggcaaaaagttcgattctagtcgcgacagaaataagccttttaagtttatgcttggaaagcaagaagtcatccgcgggtgggaggaaggcgttgctcaaatgtctgtaggtcaacgagctaaacttaccatttctccagattacgcatacggtgcgacggggcacggatctggctcaggatctggtgtaaaggatctgctccaagcctgggatctttattatcatgtgtttcgccgaatcagtggccctccgggcccaggaagcggactgtggcatgaaatgtggcacgagggtcttgaggaggcgagtagactgtatttcggtgagagaaatgttaaggggatgttcgaggtgctggagccgctgcacgccatgatggaaaggggcccacaaacgcttaaagaaaccagcttcaatcaggcatacggcagggatttgatggaggctcaggaatggtgtcgaaaatacatgaaatcaggtagctcaggtgggagcgggagtgggataattcctccccacgcgactctcgtgttcgacgtagaattgcttaaactggaaggcagcggctacgaagatgaggaagagttctacaatgatatgcaaactatcacactgaaatctgagctgaactag

SEQ ID NO: 68 - DNA, 1452 bp, Artificial
  (mCherry-) LOV2Jalpha-AcrIIA4 (#8), encoding
atggccgcagcaaagcgcgtgaagctggacgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggatctggaggttcaggtggaagtttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgacgaagcggcgaaggagttgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaactag

SEQ ID NO: 69 - DNA, 1452 bp, Artificial
  (mCherry-) LOV2Jalpha-AcrIIA4A(#41), encoding
atggccgcagcaaagcgcgtgaagctggacgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggatctggaggttcaggtggaagtttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgatgaactggctaaagcacttaatataaatgacctgataagggaaataaagaataaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaactag

SEQ ID NO: 70 - DNA, 1452 bp, Artificial
  (mCherry-) LOV2Jalpha-AcrIIA4  (#51), encoding
atggccgcagcaaagcgcgtgaagctggacgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggatctggaggttcaggtggaagtttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgaagaactcctgaagagacttaacataaacgatctcataagagagataaagaataaagactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaactag

SEQ ID NO: 71 - DNA, 1446 bp, Artificial
  (mCherry-) LOV2Jalpha-AcrIIA4(#53-41), encoding
atggccgcagcaaagcgcgtgaagctggacgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggatctggaggttcaggtggaagtttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgatgaactggctaaagcacttaacgacctcatccgcgagatcaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaactag

SEQ ID NO: 72 - DNA, 1455 bp, Artificial
  (mCherry-) LOV2Jalpha-AcrIIA4A  (#69-41), encoding
atggccgcagcaaagcgcgtgaagctggacgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggatctggaggttcaggtggaagtttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgatgaactggctaaagcacttgccaacattaacgacctcatccgcgagatcaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaactag

SEQ ID NO: 73 - DNA, 1458 bp, Artificial
  (mCherry-) LOV2Jalpha-AcrIIA4A  (#71-51), encoding
atggccgcagcaaagcgcgtgaagctggacgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggatctggaggttcaggtggaagtttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgaagaactcctgaagagacttgggcctaacatcaatgatttgattagagaaattaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaactag

SEQ ID NO: 74 - DNA, 1500 bp, Artificial
  cMyc NLS-AcrIIA4-mCherry-LEXY (pl76), encoding
atgcccgcagcaaagcgcgtgaagctggacatgggtggatcaaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaacggcgggtctggtggttctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggatctggaggttcaggtggaagtttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgacgagctgctgaaggagctggccgacctgaacctggactag

SEQ ID NO: 75 - DNA, 309 bp, Artificial
  SV40 NLS AcrIIA4 , encoding
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaactag

SEQ ID NO: 76 - DNA, 1023 bp, Artificial
  cMycP1A NLS-mCherry-AcrIIA4, encoding
atggccgcagcaaagcgcgtgaagctggacgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggatctggaggttcaggtggaagtaatattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccaggagtacgaagatgaggaagagttctacaatgatatgcaaactatcacactgaaatctgagctgaactag

SEQ ID NO: 77 - DNA, 4848 bp, Artificial
  dCas9-VP64 transactivation luciferase reporter plasmid
gacgatatccctcgatcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacgctatggcatgcatgtgtcgcgacctgcaggccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagagctagccctatataagcagagctcgtttagtgaaccgtcagatcgcctggagacgccatccacgctgttttgacctccatagaagacaccgggaccgatccagcctccgcggccccggtaccgaattcgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagtaaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgtc

SEQ ID NO: 78 - protein, 245 aa, Artificial
  AsLOV2 AcrIIA4 insertion between G62/W63
MAPKKKRKVGGSGGSMNINDLIREIKNKDYTVKLSGTDSNSITQLIIRVNNDGNEYVISESENESIVEKFISAFKNGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELWNQEYEDEEEFYNDMQTITLKSELN

SEQ ID NO: 79 - protein, 245 aa, Artificial
  AsLOV2 AcrIIA4 insertion between W63/N64
MAPKKKRKVGGSGGSMNINDLIREIKNKDYTVKLSGTDSNSITQLIIRVNNDGNEYVISESENESIVEKFISAFKNGWLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELNQEYEDEEEFYNDMQTITLKSELN

SEQ ID NO: 80 - protein, 245 aa, Artificial
  AsLOV2 AcrIIA4 insertion between N64/Q65
MAPKKKRKVGGSGGSMNINDLIREIKNKDYTVKLSGTDSNSITQLIIRVNNDGNEYVISESENESIVEKFISAFKNGWNLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELQEYEDEEEFYNDMQTITLKSELN

SEQ ID NO: 81 - protein, 245 aa, Artificial
  AsLOV2 AcrIIA4 insertion between Q65/E66
MAPKKKRKVGGSGGSMNINDLIREIKNKDYTVKLSGTDSNSITQLIIRVNNDGNEYVISESENESIVEKFISAFKNGWNQLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELEYEDEEEFYNDMQTITLKSELN

SEQ ID NO: 82 - protein, 245 aa, Artificial
  AsLOV2 AcrIIA4 insertion between E66/Y67
MAPKKKRKVGGSGGSMNINDLIREIKNKDYTVKLSGTDSNSITQLIIRVNNDGNEYVISESENESIVEKFISAFKNGWNQELATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELYEDEEEFYNDMQTITLKSELN

SEQ ID NO: 83 - protein, 245 aa, Artificial
  AsLOV2 AcrIIA4 insertion between Y67/E68
MAPKKKRKVGGSGGSMNINDLIREIKNKDYTVKLSGTDSNSITQLIIRVNNDGNEYVISESENESIVEKFISAFKNGWNQEYLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELEDEEEFYNDMQTITLKSELN

SEQ ID NO: 84 - protein, 245 aa, Artificial
  AsLOV2 AcrIIA4 insertion between E68/D69
MAPKKKRKVGGSGGSMNINDLIREIKNKDYTVKLSGTDSNSITQLIIRVNNDGNEYVISESENESIVEKFISAFKNGWNQEYELATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELDEEEFYNDMQTITLKSELN

SEQ ID NO: 85 - protein, 245 aa, Artificial
  AsLOV2 AcrIIA4 insertion between E85/L86
MAPKKKRKVGGSGGSMNINDLIREIKNKDYTVKLSGTDSNSITQLIIRVNNDGNEYVISESENESIVEKFISAFKNGWNQEYEDEEEFYNDMQTITLKSELATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELLN

SEQ ID NO: 86 - protein, 245 aa, Artificial
  AsLOV2 AcrIIA4 insertion between L86/N87
MAPKKKRKVGGSGGSMNINDLIREIKNKDYTVKLSGTDSNSITQLIIRVNNDGNEYVISESENESIVEKFISAFKNGWNQEYEDEEEFYNDMQTITLKSELLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELN

SEQ ID NO: 87 - protein, 245 aa, Artificial
  AsLOV2 AcrIIA4 insertion after N87
MAPKKKRKVGGSGGSMNINDLIREIKNKDYTVKLSGTDSNSITQLIIRVNNDGNEYVISESENESIVEKFISAFKNGWNQEYEDEEEFYNDMQTITLKSELNLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKEL

SEQ ID NO: 88 - protein, 245 aa, Artificial; identical to SEQ ID NO: 82
  GWNQE[LOV2]YED - (#1)

SEQ ID NO: 89 - protein, 247 aa, Artificial
  GWNQEG[LOV2]GYED - (#2)
MAPKKKRKVGGSGGSMNINDLIREIKNKDYTVKLSGTDSNSITQLIIRVNNDGNEYVISESENESIVEKFISAFKNGWNQEGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGYEDEEEFYNDMQTITLKSELN

SEQ ID NO: 90 - protein, 249 aa, Artificial
  GWNQESG[LOV2]GSYED - (#3)
MAPKKKRKVGGSGGSMNINDLIREIKNKDYTVKLSGTDSNSITQLIIRVNNDGNEYVISESENESIVEKFISAFKNGWNQESGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGSYEDEEEFYNDMQTITLKSELN

SEQ ID NO: 91 - protein, 244 aa, Artificial
  GWNQ[LOV2]YED - (#4)
MAPKKKRKVGGSGGSMNINDLIREIKNKDYTVKLSGTDSNSITQLIIRVNNDGNEYVISESENESIVEKFISAFKNGWNQLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELYEDEEEFYNDMQTITLKSELN

SEQ ID NO: 92 - protein, 246 aa, Artificial
  GWNQG[LOV2]GYED - (#5)
MAPKKKRKVGGSGGSMNINDLIREIKNKDYTVKLSGTDSNSITQLIIRVNNDGNEYVISESENESIVEKFISAFKNGWNQGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGYEDEEEFYNDMQTITLKSELN

SEQ ID NO: 93 - protein, 248 aa, Artificial
  GWNQSG[LOV2]GSYED - (#6)
MAPKKKRKVGGSGGSMNINDLIREIKNKDYTVKLSGTDSNSITQLIIRVNNDGNEYVISESENESIVEKFISAFKNGWNQSGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGSYEDEEEFYNDMQTITLKSELN

SEQ ID NO: 94 - protein, 243 aa, Artificial
  GWN[LOV2]YED  - (#7)
MAPKKKRKVGGSGGSMNINDLIREIKNKDYTVKLSGTDSNSITQLIIRVNNDGNEYVISESENESIVEKFISAFKNGWNLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELYEDEEEFYNDMQTITLKSELN

SEQ ID NO: 95 - protein, 245 aa, Artificial
  GWNG[LOV2]GYED - (#8)
MAPKKKRKVGGSGGSMNINDLIREIKNKDYTVKLSGTDSNSITQLIIRVNNDGNEYVISESENESIVEKFISAFKNGWNGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGYEDEEEFYNDMQTITLKSELN

SEQ ID NO: 96 - protein, 247 aa, Artificial
  GWNSG[LOV2]GSYED - (#9)
MAPKKKRKVGGSGGSMNINDLIREIKNKDYTVKLSGTDSNSITQLIIRVNNDGNEYVISESENESIVEKFISAFKNGWNSGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGSYEDEEEFYNDMQTITLKSELN

SEQ ID NO: 97 - protein, 249 aa, Artificial
  GWNSGG[LOV2]GGSYED - (#10)
MAPKKKRKVGGSGGSMNINDLIREIKNKDYTVKLSGTDSNSITQLIIRVNNDGNEYVISESENESIVEKFISAFKNGWNSGGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGGSYEDEEEFYNDMQTITLKSELN

SEQ ID NO: 98 - protein, 249 aa, Artificial
  GWNGSG[LOV2]GSGYED - (#11)
MAPKKKRKVGGSGGSMNINDLIREIKNKDYTVKLSGTDSNSITQLIIRVNNDGNEYVISESENESIVEKFISAFKNGWNGSGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGSGYEDEEEFYNDMQTITLKSELN

SEQ ID NO: 99 - protein, 257 aa, Artificial
  GWNGGSGGSG[LOV2]GSGGSGGYED - (#12)
MAPKKKRKVGGSGGSMNINDLIREIKNKDYTVKLSGTDSNSITQLIIRVNNDGNEYVISESENESIVEKFISAFKNGWNGGSGGSGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGSGGSGGYEDEEEFYNDMQTITLKSELN

SEQ ID NO: 100 - protein, 258 aa, Artificial
  GWNQGGSGGSG[LOV2]GSGGSGGYED - (#13)
MAPKKKRKVGGSGGSMNINDLIREIKNKDYTVKLSGTDSNSITQLIIRVNNDGNEYVISESENESIVEKFISAFKNGWNQGGSGGSGLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELGSGGSGGYEDEEEFYNDMQTITLKSELN

SEQ ID NO: 101 - protein, 300 aa, Artificial
  GWNQE[UniRap]YED - (#Rap1)
MAPKKKRKVGGSGGSMNINDLIREIKNKDYTVKLSGTDSNSITQLIIRVNNDGNEYVISESENESIVEKFISAFKNGWNQETCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLEYEDEEEFYNDMQTITLKSELN

SEQ ID NO: 102 - protein, 299 aa, Artificial
  GWNQ[UniRap]YED - (#Rap2)
MAPKKKRKVGGSGGSMNINDLIREIKNKDYTVKLSGTDSNSITQLIIRVNNDGNEYVISESENESIVEKFISAFKNGWNQTCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLEYEDEEEFYNDMQTITLKSELN

SEQ ID NO: 103 - protein, 298 aa, Artificial
  GWN[UniRap]YED - (#Rap3)
MAPKKKRKVGGSGGSMNINDLIREIKNKDYTVKLSGTDSNSITQLIIRVNNDGNEYVISESENESIVEKFISAFKNGWNTCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLEYEDEEEFYNDMQTITLKSELN

SEQ ID NO: 104 - protein, 302 aa, Artificial
  GWNSG[UniRap]GSYED - (#Rap4)
MAPKKKRKVGGSGGSMNINDLIREIKNKDYTVKLSGTDSNSITQLIIRVNNDGNEYVISESENESIVEKFISAFKNGWNSGTCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLEGSYEDEEEFYNDMQTITLKSELN

SEQ ID NO: 105 - protein, 303 aa, Artificial
  GWNQSG[UniRap]GSYED - (#Rap5)
MAPKKKRKVGGSGGSMNINDLIREIKNKDYTVKLSGTDSNSITQLIIRVNNDGNEYVISESENESIVEKFISAFKNGWNQSGTCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLEGSYEDEEEFYNDMQTITLKSELN

SEQ ID NO: 106 - protein, 306 aa, Artificial
  GWNQEGSG[UniRap]GSGYED - (#Rap6)
MAPKKKRKVGGSGGSMNINDLIREIKNKDYTVKLSGTDSNSITQLIIRVNNDGNEYVISESENESIVEKFISAFKNGWNQEGSGTCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLEGSGYEDEEEFYNDMQTITLKSELN

SEQ ID NO: 107 - protein, 304 aa, Artificial
  GWNGSG[UniRap]GSGYED - (#Rap7)
MAPKKKRKVGGSGGSMNINDLIREIKNKDYTVKLSGTDSNSITQLIIRVNNDGNEYVISESENESIVEKFISAFKNGWNGSGTCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLEGSGYEDEEEFYNDMQTITLKSELN

SEQ ID NO: 108 - protein, 483 aa, Artificial
  (mCherry-) LOV2Jalpha-AcrIIA4Aalpha (#8)
MAAAKRVKLDVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSGGSGGSLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELNINDLIREIKNKDYTVKLSGTDSNSITQLIIRVNNDGNEYVISESENESIVEKFISAFKNGWNQEYEDEEEFYNDMQTITLKSELN

SEQ ID NO: 109 - protein, 483 aa, Artificial
  (mCherry-) LOV2Jalpha-AcrIIA4Aalpha (#41)
MAAAKRVKLDVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSGGSGGSLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDELAKALNINDLIREIKNKDYTVKLSGTDSNSITQLIIRVNNDGNEYVISESENESIVEKFISAFKNGWNQEYEDEEEFYNDMQTITLKSELN

SEQ ID NO: 110 - protein, 483 aa, Artificial
  (mCherry-) LOV2Jalpha-AcrIIA4Aalpha (#51)
MAAAKRVKLDVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSGGSGGSLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIEELLKRLNINDLIREIKNKDYTVKLSGTDSNSITQLIIRVNNDGNEYVISESENESIVEKFISAFKNGWNQEYEDEEEFYNDMQTITLKSELN

SEQ ID NO: 111 - protein, 481 aa, Artificial
  (mCherry-) LOV2Jalpha-AcrIIA4Aalpha (#53-41)
MAAAKRVKLDVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSGGSGGSLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDELAKALNDLIREIKNKDYTVKLSGTDSNSITQLIIRVNNDGNEYVISESENESIVEKFISAFKNGWNQEYEDEEEFYNDMQTITLKSELN

SEQ ID NO: 112 - protein, 484 aa, Artificial
  (mCherry-) LOV2Jalpha-AcrIIA4Aalpha (#69-41)
MAAAKRVKLDVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSGGSGGSLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDELAKALANINDLIREIKNKDYTVKLSGTDSNSITQLIIRVNNDGNEYVISESENESIVEKFISAFKNGWNQEYEDEEEFYNDMQTITLKSELN

SEQ ID NO: 113 - protein, 485 aa, Artificial
  (mCherry-) LOV2Jalpha-AcrIIA4Aalpha (#71-51)
MAAAKRVKLDVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSGGSGGSLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIEELLKRLGPNINDLIREIKNKDYTVKLSGTDSNSITQLIIRVNNDGNEYVISESENESIVEKFISAFKNGWNQEYEDEEEFYNDMQTITLKSELN

SEQ ID NO: 114 - protein, 499 aa, Artificial
  cMyc NLS-AcrIIA4-mCherry-LEXY (pl76)
MPAAKRVKLDMGGSNINDLIREIKNKDYTVKLSGTDSNSITQLIIRVNNDGNEYVISESENESIVEKFISAFKNGWNQEYEDEEEFYNDMQTITLKSELNGGSGGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSGGSGGSLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDELLKELADLNLD

SEQ ID NO: 115 - protein, 102 aa, Artificial
  SV40 NLS AcrIIA4
MAPKKKRKVGGSGGSMNINDLIREIKNKDYTVKLSGTDSNSITQLIIRVNNDGNEYVISESENESIVEKFISAFKNGWNQEYEDEEEFYNDMQTITLKSELN

SEQ ID NO: 116 - protein, 340 aa, Artificial
  cMycP1A NLS-mCherry-AcrIIA4
MAAAKRVKLDVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSGGSGGSNINDLIREIKNKDYTVKLSGTDSNSITQLIIRVNNDGNEYVISESENESIVEKFISAFKNGWNQEYEDEEEFYNDMQTITLKSELN

SEQ ID NO: 117 - DNA, 14547 bp, Artificial
  dCas9-VP64-2A-GFP reporter plasmid
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgagccccaagaagaagagaaaggtggaggccagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacgctagcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcggggggggggggggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 118 - DNA, 3226 bp, Artificial
  TetO-targeting gRNA for dCas9-VP64 reporter plasmid
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttatcgtaacttgaaagtatttcgatttcttggctttatatatcttgtggaaaggacgaaacaccgtctctatcactgatagggaggttttagagctagaaatagcaagttaaaataaggctagtccgttatcaacttgaaaaagtggcaccgagtcggtgctttttttccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 119 - DNA, 7601 bp, Artificial
  Cas9 Luciferase Cleavage Reporter Plasmid
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctaggcgatcgctcgagcccgggaattcgtttaaacctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatactagtgcaaaaaaagcaccgactcggtgccactttttcaagttgataacggactagccttatttaaacttgctatgctgtttccagcatagctcttaaacggtagtcggtcttagagtccggtgtaccgtggtctcatacagaacttataagattcccaaatccaaagacatttcacgtttatggtgatttcccagaacacatagcgacatgcaaatatgggcatcgataaactagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 120 - DNA, 429 bp, Artificial
  LOV2 encoding alternative codon usage
ttggctactacacttgaacgtattgagaagaactttgtcattactgacccaagattgccagataatcccattatattcgcgtccgatagtttcttgcagttgacagaatatagccgtgaagaaattttgggaagaaactgcaggtttctacaaggtcctgaaactgatcgcgcgacagtgagaaaaattagagatgccatagataaccaaacagaggtcactgttcagctgattaattatacaaagagtggtaaaaagttctggaacctctttcacttgcagcctatgcgagatcagaagggagatgtccagtactttattggggttcagttggatggaactgagcatgtccgagatgctgccgagagagagggagtcatgctgattaagaaaactgcagaaaatattgacgaagcggcgaaggagttg

SEQ ID NO: 121 - protein, 5 aa, Artificial
  fusion site corresponding to amino acids 62 to 69 of ACRIIA4, variant #4-Fig.7
  MISC_FEATURE (2)..(3): site of receptor domain insertion
GWYED

SEQ ID NO: 122 - protein, 11 aa, Artificial
  fusion site corresponding to amino acids 62 to 69 of ACRIIA4, variant #5-Fig.7
  MISC_FEATURE (1)..(2): site of receptor domain insertion
GGSGGSGGYED

SEQ ID NO: 123 - protein, 7 aa, Artificial
  fusion site corresponding to amino acids 62 to 69 of ACRIIA4, variant #6-Fig.7
  MISC_FEATURE (3)..(4): site of receptor domain insertion
GWGGYED

SEQ ID NO: 124 - protein, 9 aa, Artificial
  fusion site corresponding to amino acids 62 to 69 of ACRIIA4, variant #7-Fig.7
  MISC_FEATURE (4)..(5): site of receptor domain insertion
GWSGGSYED

SEQ ID NO: 125 - protein, 11 aa, Artificial
  fusion site corresponding to amino acids 62 to 69 of ACRIIA4, variant #8-Fig.7
  MISC_FEATURE (5)..(6): site of receptor domain insertion
GWGSGGSGYED

SEQ ID NO: 126 - protein, 13 aa, Artificial
  fusion site corresponding to amino acids 62 to 69 of ACRIIA4, variant #9-Fig.7
  MISC_FEATURE (6)..(7): site of receptor domain insertion
GWGGSGGSGGYED

SEQ ID NO: 127 - protein, 15 aa, Artificial
  fusion site corresponding to amino acids 62 to 69 of ACRIIA4, variant #10-Fig.7
  MISC_FEATURE (7)..(8): site of receptor domain insertion
GWSGGSGGSGGSYED

SEQ ID NO: 128 - protein, 17 aa, Artificial
  fusion site corresponding to amino acids 62 to 69 of ACRIIA4, variant #11-Fig.7
  MISC_FEATURE (8)..(9): site of receptor domain insertion
GWGSGGSGGSGGSGYED

SEQ ID NO: 129 - protein, 19 aa, Artificial
  fusion site corresponding to amino acids 62 to 69 of ACRIIA4, variant #12-Fig.7
  MISC_FEATURE (9)..(10): site of receptor domain insertion
GWGGSGGSGGSGGSGGYED

SEQ ID NO: 130 - protein, 8 aa, Artificial
  fusion site corresponding to amino acids 62 to 69 of ACRIIA4, variant #13-Fig.7
  MISC_FEATURE (4)..(5): site of receptor domain insertion
GWNGGYED

SEQ ID NO: 131 - protein, 10 aa, Artificial
  fusion site corresponding to amino acids 62 to 69 of ACRIIA4, variant #14-Fig.7
  MISC_FEATURE (5)..(6): site of receptor domain insertion
GWNSGGSYED

SEQ ID NO: 132 - protein, 12 aa, Artificial
  fusion site corresponding to amino acids 62 to 69 of ACRIIA4, variant #15-Fig.7
  MISC_FEATURE (6)..(7): site of receptor domain insertion
GWNGSGGSGYED

SEQ ID NO: 133 - protein, 13 aa, Artificial
  fusion site corresponding to amino acids 62 to 69 of ACRIIA4, variant #16-Fig.7
  MISC_FEATURE (7)..(8): site of receptor domain insertion
GWNQGSGGSGYED

SEQ ID NO: 134 - DNA, 738 bp, Artificial; identical to SEQ ID NO: 42
  AcrIIA4-LOV2 hybrid 1

SEQ ID NO: 135 - DNA, 735 bp, Artificial; identical to SEQ ID NO: 51
  AcrIIA4-LOV2 hybrid 2

SEQ ID NO: 136 - DNA, 732 bp, Artificial; identical to SEQ ID NO: 54
  AcrIIA4-LOV2 hybrid 3

SEQ ID NO: 137 - DNA, 729 bp, Artificial
  AcrIIA4-LOV2 hybrid 4
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 138 - DNA, 747 bp, Artificial
  AcrIIA4-LOV2 hybrid 5
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggcagcggcggctacgaagatgaggaagagttctacaatgatatgcaaactatcacactgaaatctgagctgaactag

SEQ ID NO: 139 - DNA, 735 bp, Artificial
  AcrIIA4-LOV2 hybrid 6
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggctacgaagatgaggaagagttctacaatgatatgcaaactatcacactgaaatctgagctgaactag

SEQ ID NO: 140 - DNA, 741 bp, Artificial
  AcrIIA4-LOV2 hybrid 7
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagctacgaagatgaggaagagttctacaatgatatgcaaactatcacactgaaatctgagctgaactag

SEQ ID NO: 141 - DNA, 747 bp, Artificial
  AcrIIA4-LOV2 hybrid 8
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggctacgaagatgaggaagagttctacaatgatatgcaaactatcacactgaaatctgagctgaactag

SEQ ID NO: 142 - DNA, 753 bp, Artificial
  Atggccccgaagaaaaagcgaaaggtgggcggaagcggaggttccat gaacattaacgacctgattcgggaaataaagaacaaggactacacggtcaagctgtcaggcaccga ctccaactctataacacagctcattataagggttaataatgatggcaacgaatatgtcatctctga atccgagaatgaatcaatcgtggagaaattcatcagtgcatttaaaaacggctggggcggcagcgg ccttgctacaacttt
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggggcggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggctacgaagatgaggaagagttctacaatgatatgcaaactatcacactgaaatctgagctgaactag

SEQ ID NO: 143 - DNA, 759 bp, Artificial
  AcrIIA4-LOV2 hybrid 10
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggagcggcggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggcagctacgaagatgaggaagagttctacaatgatatgcaaactatcacactgaaatctgagctgaactag

SEQ ID NO: 144 - DNA, 765 bp, Artificial
  AcrIIA4-LOV2 hybrid 11
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggggcagcggcggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggcagcggctacgaagatgaggaagagttctacaatgatatgcaaactatcacactgaaatctgagctgaactag

SEQ ID NO: 145 - DNA, 771 bp, Artificial
  AcrIIA4-LOV2 hybrid 12
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggggcggcagcggcggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggcagcggcggctacgaagatgaggaagagttctacaatgatatgcaaactatcacactgaaatctgagctgaactag

SEQ ID NO: 146 - DNA, 738 bp, Artificial; identical to SEQ ID NO: 55
  AcrIIA4-LOV2 hybrid 13

SEQ ID NO: 147 - DNA, 744 bp, Artificial; identical to SEQ ID NO: 56
  AcrIIA4-LOV2 hybrid 14

SEQ ID NO: 148 - DNA, 750 bp, Artificial; identical to SEQ ID NO: 58
  AcrIIA4-LOV2 hybrid 15

SEQ ID NO: 149 - DNA, 753 bp, Artificial
  AcrIIA4-LOV2 hybrid 16
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccagggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggctacgaagatgaggaagagttctacaatgatatgcaaactatcacactgaaatctgagctgaactag

SEQ ID NO: 150 - DNA, 729 bp, Artificial
  CASANOVA (LOVC450A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaatgctagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 151 - DNA, 732 bp, Artificial
  Acr-LOV hybrid 3 (LOVC450A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccttgctacaactttggagcggattgagaagaatttcgtcataactgatcccaggttgcctgataatcctataatattcgcctccgatagttttcttcaacttacggagtactccagagaggagatactgggaaggaatgctagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 152 - DNA, 735 bp, Artificial
  Acr-LOV hybrid 2 (LOVC450A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaaccagcttgctacaactttggagcggattgagaagaatttcgtcataactgatcccaggttgcctgataatcctataatattcgcctccgatagttttcttcaacttacggagtactccagagaggagatactgggaaggaatgctagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 153 - DNA, 771 bp, Artificial
  Acr-LOV hybrid 12 (LOVC450A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggggcggcagcggcggcagcggccttgctacaactttggagcggattgagaagaatttcgtcataactgatcccaggttgcctgataatcctataatattcgcctccgatagttttcttcaacttacggagtactccagagaggagatactgggaaggaatgctagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggcagcggcggctacgaagatgaggaagagttctacaatgatatgcaaactatcacactgaaatctgagctgaactag

SEQ ID NO: 154 - DNA, 753 bp, Artificial
  Acr-LOV hybrid 9 (LOVC450A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggggcggcagcggccttgctacaactttggagcggattgagaagaatttcgtcataactgatcccaggttgcctgataatcctataatattcgcctccgatagttttcttcaacttacggagtactccagagaggagatactgggaaggaatgctagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggctacgaagatgaggaagagttctacaatgatatgcaaactatcacactgaaatctgagctgaactag

SEQ ID NO: 155 - DNA, 759 bp, Artificial
  Acr-LOV hybrid 10 (LOVC450A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggagcggcggcagcggccttgctacaactttggagcggattgagaagaatttcgtcataactgatcccaggttgcctgataatcctataatattcgcctccgatagttttcttcaacttacggagtactccagagaggagatactgggaaggaatgctagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggcggcagctacgaagatgaggaagagttctacaatgatatgcaaactatcacactgaaatctgagctgaactag

SEQ ID NO: 156 - DNA, 729 bp, Artificial
  CASANOVA (LOVG528A, N538E)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaagcagtgatgctgattaagaaaacagcagaggagatcgacgaagcggcgaaagagttgtacgaagatgaggaagagttctacaatgatatgcaaactatcacactgaaatctgagctgaactag

SEQ ID NO: 157 - DNA, 729 bp, Artificial
  CASANOVA (LOVI532A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctggcaaagaaaacagcagagaatatcgacgaagcggcgaaagagttgtacgaagatgaggaagagttctacaatgatatgcaaactatcacactgaaatctgagctgaactag

SEQ ID NO: 158 - DNA, 729 bp, Artificial
  CASANOVA (LOVT406/407A,G528A,N538E)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctgcagcgttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaagcagtgatgctgattaagaaaacagcagaggagatcgacgaagcggcgaaagagttgtacgaagatgaggaagagttctacaatgatatgcaaactatcacactgaaatctgagctgaactag

SEQ ID NO: 159 - DNA, 729 bp, Artificial
  CASANOVA (LOVT406/407A,I532A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctgcagcgttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctggcaaagaaaacagcagagaatatcgacgaagcggcgaaagagttgtacgaagatgaggaagagttctacaatgatatgcaaactatcacactgaaatctgagctgaactag

SEQ ID NO: 160 - DNA, 729 bp, Artificial
  CASANOVA (LOVT406/407A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctgcagcgttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 161 - DNA, 750 bp, Artificial
  Acr-LOV hybrid 15 (LOVG528A, N538E)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaagcagtgatgctgattaagaaaacagcagaggagatcgacgaagcggcgaaagagttgggcagcggctacgaagatgaggaagagttctacaatgatatgcaaactatcacactgaaatctgagctgaactag

SEQ ID NO: 162 - DNA, 750 bp, Artificial
  Acr-LOV hybrid 15 (LOVI532A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctggcaaagaaaacagcagagaatatcgacgaagcggcgaaagagttgggcagcggctacgaagatgaggaagagttctacaatgatatgcaaactatcacactgaaatctgagctgaactag

SEQ ID NO: 163 - DNA, 750 bp, Artificial
  Acr-LOV hybrid 15 (LOVT406/407A,G528A,N538E)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacggcagcggccttgctgcagcgttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaagcagtgatgctgattaagaaaacagcagaggagatcgacgaagcggcgaaagagttgggcagcggctacgaagatgaggaagagttctacaatgatatgcaaactatcacactgaaatctgagctgaactag

SEQ ID NO: 164 - DNA, 750 bp, Artificial
  Acr-LOV hybrid 15 (LOVT406/407A,I532A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacggcagcggccttgctgcagcgttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctggcaaagaaaacagcagagaatatcgacgaagcggcgaaagagttgggcagcggctacgaagatgaggaagagttctacaatgatatgcaaactatcacactgaaatctgagctgaactag

SEQ ID NO: 165 - DNA, 750 bp, Artificial
  Acr-LOV hybrid 15 (LOVT406/407A)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaacggcagcggccttgctgcagcgttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggctacgaagatgaggaagagttctacaatgatatgcaaactatcacactgaaatctgagctgaactag

SEQ ID NO: 166 - DNA, 750 bp, Artificial
  Acr-LOV hybrid 15 (AcrN64K)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggaagggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggctacgaagatgaggaagagttctacaatgatatgcaaactatcacactgaaatctgagctgaactag

SEQ ID NO: 167 - DNA, 750 bp, Artificial
  Acr-LOV hybrid 15 (AcrN64R)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaatccgagaatgaatcaatcgtggagaaattcatcagtgcatttaaaaacggctggagaggcagcggccttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgggcagcggctacgaagatgaggaagagttctacaatgatatgcaaactatcacactgaaatctgagctgaactag

SEQ ID NO: 168 - DNA, 729 bp, Artificial
  CASANOVA (AcrE45K)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctaagtccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 169 - DNA, 729 bp, Artificial
  CASANOVA (AcrK18Q)
atggccccgaagaaaaagcgaaaggtgggcggaagcggaggttccatgaacattaacgacctgattcgggaaataaagaacaaggactacacggtccagctgtcaggcaccgactccaactctataacacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 170 - DNA, 729 bp, Artificial
  CASANOVA (AcrS46D)
atggccccgaagaaaaagcgaaaggtgggcggaagcggaggttccatgaacattaacgacctgattcgggaaataaagaacaaggactacacggtcaagctgtcaggcaccgactccaactctataacacagctcattataagggttaataatgatggcaacgaatatgtcatctctgaagacgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 171 - DNA, 729 bp, Artificial
  CASANOVA (AcrT16F)
atggccccgaagaaaaagcgaaaggtgggcggaagcggaggttccatgaacattaacgacctgattcgggaaataaagaacaaggactacttcgtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 172 - DNA, 729 bp, Artificial
  CASANOVA (AcrT16Y)
atggccccgaagaaaaagcgaaaggtgggcggaagcggaggttccatgaacattaacgacctgattcgggaaataaagaacaaggactactatgtcaagctgtcaggcaccgactccaactctataacacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 173 - DNA, 729 bp, Artificial
  CASANOVA (AcrT22H)
atggccccgaagaaaaagcgaaaggtgggcggaagcggaggttccatgaacattaacgacctgattcgggaaataaagaacaaggactacacggtcaagctgtcaggccacgactccaactctataacacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 174 - DNA, 729 bp, Artificial
  CASANOVA (AcrT28E)
atggccccgaagaaaaagcgaaaggtgggcggaagcggaggttccatgaacattaacgacctgattcgggaaataaagaacaaggactacacggtcaagctgtcaggcaccgactccaactctatagaacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 175 - DNA, 729 bp, Artificial
  CASANOVA (AcrT28N)
atggccccgaagaaaaagcgaaaggtgggcggaagcggaggttccatgaacattaacgacctgattcgggaaataaagaacaaggactacacggtcaagctgtcaggcaccgactccaactctataaaccagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

SEQ ID NO: 176 - DNA, 729 bp, Artificial
  CASANOVA (AcrT28Q)
atggccccgaagaaaaagcgaaaggtgggcggaagcggaggttccatgaacattaacgacctgattcgggaaataaagaacaaggactacacggtcaagctgtcaggcaccgactccaactctatacaacagctcattataagggttaataatgatggcaacgaatatgtcatctctgaatccgagaatgaatcaatcgtggagaaattcatcagtgcatttaaaaacggctggcttgctacaactttggagcggattgagaagaatttcgtcataactgatcccaggttgcctgataatcctataatattcgcctccgatagttttcttcaacttacggagtactccagagaggagatactgggaaggaattgcagattcctccaaggtccagaaactgatagagctactgtgaggaagatacgggatgccattgacaatcaaactgaagtgacagtgcaacttatcaattacaccaagtcagggaaaaaattctggaacctgttccatttgcaacccatgagggaccaaaagggcgatgtgcaatatttcataggcgttcaacttgatggtactgaacacgtcagagatgcggcggagcgagaaggagtgatgctgattaagaaaacagcagagaatatcgacgaagcggcgaaagagttgtacgaagatgaggaagagttctacaatgatatgcaaactatcacactgaaatctgagctgaactag

(19) United States
(12) Patent Application Publication
(10) Pub. No.: US 2021/0198328 A1
(43) Pub. Date: Jul. 1, 2021

(54) MODULATED CAS-INHIBITORS

(71) Applicants: Universität Heidelberg, Heidelberg (DE); Deutsches Krebsforschungszentrum, Heidelberg (DE)

(72) Inventors: Dominik Niopek, Heidelberg (DE); Roland Eils, Schriesheim (DE); Felix Bubeck, Heidelberg (DE); Dirk Grimm, Heidelberg (DE); Mareike Daniela Hoffmann, Dossenheim (DE); Max Christian Waldhauer, Heidelberg (DE); Laura Dietz, Heidelberg (DE); Julia Fakhiri, Heidelberg (DE); Andreas Bietz, Heidelberg (DE)

(21) Appl. No.: 16/756,887

(22) PCT Filed: Oct. 5, 2018
(86) PCT No.: PCT/EP2018/077164

§ 371 Date: Apr. 17, 2020

(30) Foreign Application Priority Data
Oct. 17, 2017 (EP) 17196813.4

(51) Int. Cl. C07K 14/47; C12N 9/22; C12N 15/113

(52) U.S. Cl. CPC C07K 14/47; C12N 9/22; A61K 38/00; C07K 2319/70; C12N 15/113

(57) ABSTRACT

The present invention relates to a polynucleotide encoding a fusion polypeptide comprising an anti-CRISPR (Acr) polypeptide, wherein said fusion polypeptide further comprises a receptor domain changing conformation upon reception of a stimulus. The present invention also relates to a vector comprising the polynucleotide of the present invention, to a bipartite Acr polypeptide comprising a first partial Acr polypeptide comprising amino acids corresponding to amino acids 10 to 62 of SEQ ID NO: 1, and a second partial Acr polypeptide comprising amino acids corresponding to amino acids 67 to 77 of SEQ ID NO: 1, and to a host cell comprising the aforesaid polynucleotide compounds. The present invention also relates to the said compounds for use in medicine, in particular for use in treatment and/or prevention of genetic disease, neurodegenerative disease, cancer, and/or infectious disease. Moreover, the present invention also relates to a kit, methods, and uses related thereto.

The present invention relates to a polynucleotide encoding a fusion polypeptide comprising an anti-CRISPR (Acr) polypeptide, wherein stud fusion polypeptide further comprises a receptor domain changing conformation upon reception of a stimulus. The present invention also relates to a vector comprising the polynucleotide of the present invention, to a bipartite Acr polypeptide comprising a first partial Acr polypeptide comprising amino acids corresponding to amino acids 10 to 62 of SEQ ID NO: 1, and a second partial Acr polypeptide comprising amino acids corresponding to amino acids 67 to 77 of SEQ ID NO: 1, and to a host cell comprising the aforesaid polynucleotide compounds. The present invention also relates to the said compounds for use in medicine, in particular for use in treatment and/or prevention of genetic disease, neurodegenerative disease, cancer, and/or infectious disease. Moreover, the present invention also relates to kits, methods, and uses related thereto.

CRISPR (Clustered, Regularly Interspaced Short Palindromic Repeats) systems in bacteria and archaea mediate specific degradation of foreign, invading nucleic acids (Barrangou et al., 2007; Bhaya et al., 2011; Terns and Terns, 2011: Wiedenheft et al., 2012). They comprise a CRISPR-associated (Cas) nuclease which can be programmed by short guideRNAs (gRNAs) to induce double-strand breaks at specific, sequence-complementary DNA bet (Jinek et al., 2012). Diverse CRISPR-Cas systems have been adopted for genome engineering in mammalian cells and animals, most prominently the CRISPR-Cas9 system from Streptococcus pyogenes (SpyCas9) (Cong et al., 2013; Jinek et al., 2013; Mali et al., 2013). CRISPR-Cas9 systems have also been successfully applied for genome editing in embryonic stem cells (Wang et al., 2013) as well as in animals. For instance, transgenic mice were reported that stably express SpyCas9 and thus enable in vivo gene knockout screens (Platt et al., 2014). Alternatively, transient and efficient in vivo delivery of the Cas protein and gRNA components via e.g. hydrodynamic plasmid DNA injection (Yin et al., 2014) or Adeno-associated viral (AAV) vectors (Senis et al., 2014; Ran et al., 2015) has also been achieved. Not surprisingly, the potential of CRISPR-Cas-based human gene therapy is considered to be enormous and motivates an ever increasing number of preclinical studies for treatment of genetic diseases (Schmidt and Grimm, 2015: Dai et al., 2016; Xue et al., 2016).

To enable activation or inactivation by providing an exogenous stimulus to a cell, tissue, or individual, a number of engineered SpyCas9 variants dependent on exogenous triggers have been reported. These include variants dependent on chemical triggers such as rapamycin and 4-hydroxytamoxifen (Zetsche et al., 2015; Oakes et al., 2016; Maji et al., 2017) or light (Nihongaki et al., 2015a; Nihongaki et al., 2015b; Polstein and Gersbach, 2015). In these systems, the SpyCas9 itself is modified either by insertion of a receptor or by splitting Cas9 into two pans fused to inducible dimerization domains. As alternatives, guideRNAs protected by photocleavable groups have been developed, which are activated upon exposure to 365 nm UV light (Jain et al., 2016). GuideRNAs can also be expressed front Tet operator-dependent Pol-III promoter variants to control Cas9 activity with doxycycline (de Solis et al., 2016). However, each of these aforementioned strategies requires that users adapt their particular CRISPR-Cas-based system, e.g. by exchanging the “regular” Cas9 with the engineered, inducible Cas9 variants. Depending on the robustness of the underlying approach tor Cas9 conditional activation, this can be laborious, time-consuming and expensive. Furthermore, it is at least very hard if not impossible to apply any of the existing tools for Cas9 control by exogenous triggers to previously generated, established CRISPR-Cas9 transgenic cell lines or animals expressing in which a (d)Cas9-based system has already been adapted to the particular setting/experimental condition.

There is, thus, a need in the art for improved means and methods for providing Cas nuclease activity in a cell- and/or tissue-specific manner. This problem is solved by the means and methods disclosed herein.

Accordingly, the present invention relates to a polynucleotide encoding a fusion polypeptide, said fusion polypeptide comprising an anti-CRISPR (Acr) polypeptide and a receptor domain, wherein said receptor domain changes conformation upon reception of a stimulus.

As used in the following, the terms “have”, “comprise” or “include” or any arbitrary grammatical variations thereof arc used in a non-exclusive way. Thus, these terms may both refer to a situation in which, besides the feature introduced by these terms, no further features arc present in the entity described in this context and to a situation in which one or more further features are present. As an example, the expressions “A has B”, “A comprises B” and “A includes B” may both refer to a situation in which, besides B, no other element Is present in A (i.e. a situation in which A solely and exclusively consists of B) and to a situation in which, besides B. one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms arc optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding further embodiments of the invention, without airy restrictions regarding the scope of the invention and without any restriction regarding the possibility of combining the features introduced in such way with other optional or non-optional features of the invention.

Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably 5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like, Preferably, a composition consisting essentially of a set of components will comprise less than 5% by weight, more preferably less than 3% by weight, even more preferably less than 1%, most preferably less than 0.1% by weight of non-specified components). In the context of nucleic acid sequences, the term “essentially identical” indicates a % identity value of at least 80%, preferably at least 90%, more preferably at least 98%, most preferably at least 99%. M will be understood, the term “essentially identical” includes 100%. identity. The aforesaid applies to the term “essentially complementary” mutates mutandis.

The term “polynucleotide”, as used herein, refers to a linear or circular nucleic acid molecule. The term encompasses single as well as partially or completely double-stranded polynucleotides. Preferably, the polynucleotide is RNA or DNA, including cDNA. Moreover, comprised are also chemically modified polynucleotides including naturally occurring modified polynucleotides such as glycosylated or methylated polynucleotides or artificially modified derivatives such as biotinylated polynucleotides. The polynucleotide of the present invention shall be provided, preferably, either as an isolated polynucleotide (i.e. Isolated from its natural context) or in genetically modified form.

The polynucleotide of the invention, preferably, comprises at least one heterologous sequence, i.e. comprises sequences from at least two different species. Preferably, said sequences from two different species are the sequence encoding an Acr polypeptide as specified elsewhere herein and the receptor domain. Also preferably, the polynucleotide comprises at least one heterologous sequence relative to a mammalian, preferably human, cell, i.e. comprises at least one nucleic acid sequence not known to occur or not occurring in a mammalian, preferably human, cell. Preferably, said heterologous sequence relative to a mammalian cell is at least the sequence encoding an Acr polypeptide. T he polynucleotide of the present invention has the activity of encoding a fusion polypeptide as specified elsewhere herein. Preferably, the polynucleotide comprises the nucleic acid sequence one of SHQ ID NOs: 38 to 74, more preferably 48 to 67, preferably encoding a fusion polypeptide comprising the amino acid sequence of one of SEQ ID NOs: 78 to 114, more preferably 88 to 107. As used herein, the term polynucleotide, preferably, includes variants of the specifically indicated polynucleotides. More preferably, the term polynucleotide relates to the specific polynucleotides indicated. The skilled person knows how to select a polynucleotide encoding a polypeptide having a specific amino acid sequence and also knows how to optimize the codons used in the polynucleotide according to the codon usage of the organism used for expressing said polynucleotide. The term “polynucleotide variant”, as used herein, relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the activities as specified for the specific polynucleotide. Thus, it is to be understood that a polynucleotide variant as referred to in accordance with the present invention shall have a nucleic acid sequence which differs due to at least one nucleotide substitution, deletion and/or addition. Preferably, said polynucleotide variant comprises an ortholog. a paralog or another homolog of the specific polynucleotide or of a functional subsequence thereof, e.g. of the sequence encoding an Acr polypeptide. Also preferably, said polynucleotide variant comprises a naturally occurring allele of the specific polynucleotide or of a functional subsequence thereof in particular of the sequence encoding an Acr polypeptide and/or of the receptor domain. In the context of polynucleotide variants, the term “functional subsequence”, as used herein, relates to a part of a sequence of the polynucleotide of the present invention mediating the activity as specified herein above. Polynucleotide variants also encompass polynucleotides comprising a nucleic acid sequence which is capable of hybridizing to the aforementioned specific polynucleotides or functional subsequences thereof, preferably, under stringent hybridization conditions. These stringent conditions are known to the skilled worker and can be found in Current Protocols in Molecular Biology, John Wiley & Sons, N.Y. (1989). 6.3.1-6.3.6. A preferred example for stringent hybridization conditions arc hybridization conditions in 6× sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 38° C. in aqueous buffer with a concentration of 0.1× to 5× SSC (pH 7.2). If organic solvent is present in the abovementioned buffer, for example 50% formamide. the temperature under standard conditions is approximately 42° C. The hybridization conditions for DNA:DNA hybrids are preferably, for example, 0.1× SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined, for example, for a nucleic acid with approximately 100 bp (=base pairs) in length and a G+C content of 50% in the absence of formamide. The skilled worker knows how to determine the hybridization conditions required by referring to .standard textbooks. Alternatively, polynucleotide variants are obtainable by PCR-based techniques such as mixed oligonucleotide primer-based amplification of DNA, i.e. using degenerated primers against conserved domains of a polypeptide of the present invention. Conserved domains of a polypeptide may be identified by a sequence comparison of the nucleic acid sequence of the polynucleotide or the amino acid sequence of the polypeptide of the present invention with sequences of other organisms. As a template, DNA or cDNA from bacteria, fungi, plants or, preferably, from animals may be used. Further, variants include polynucleotides comprising nucleic acid sequences which arc at least 70%, at least 75%. at least 80%. at least 85%, at least 90%, at least 95%, at least 98% or at least 99% identical to the specifically indicated nucleic acid sequences or functional subsequence thereof. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are. preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program Pilelip (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970)) and Smith and Waterman (Adv. Appl. Math. 2; 482-489 (1981))], which arc part of the GCG software packet (Genetics Computer Group, 575 Science Drive. Madison, Wisconsin, USA 53711 (1991)), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard .settings for sequence alignments. A polynucleotide comprising a fragment of any of the specifically indicated nucleic acid sequences, said polynucleotide retaining the indicated activity or activities, is also encompassed as a variant polynucleotide of the present invention. A fragment as meant herein, preferably, comprises at least 100, preferably at least 200, more preferably at least 250 consecutive nucleotides of any one of the specific nucleic acid sequences or encodes an amino acid sequence comprising at least 50, preferably at least 60, more preferably at least 75 consecutive amino acids of any one of the specific amino acid sequences.

The polynucleotides of the present invention either consist of, essentially consist of, or comprise the aforementioned nucleic acid sequences. Thus, they may contain further nucleic acid sequences as well. Specifically, the polynucleotides of the present invention may encode fusion proteins comprising further fusion partners. Such fusion proteins may comprise as additional pan polypeptides for monitoring expression (e.g.. green, yellow, blue or red fluorescent proteins, alkaline phosphatase and the like) or so called “tags” which may serve as a detectable marker or as an auxiliary measure for purification purposes. Tags for the different purposes are well known in the art and are described elsewhere herein. Preferably, the polynucleotide encodes a fusion polypeptide fused to a nuclear localization sequence (NLS) or encodes an Acr polypeptide fused to a NLS. Preferably, the polynucleotide further comprises a nucleic acid sequence encoding at least a fragment of a Cas nuclease, preferably as specified elsewhere herein; also preferably, the polynucleotide does not comprise a nucleic acid sequence encoding at least a fragment of a Cas nuclease.

Preferably, the polynucleotide is an RNA. More preferably, the polynucleotide is a DNA comprising a nucleic acid sequence expressible as a continuous RNA comprising said sequence encoding a fusion polypeptide. Preferably, in case the polynucleotide is DNA, the polynucleotide is operatively linked to expression control sequences allowing expression in prokaryotic or eukaryotic, preferably in eukaryotic host cells or isolated fractions thereof. Expression of said polynucleotide comprises transcription of the polynucleotide, preferably into a translatable mRNA, Regulatory elements ensuring expression in eukaryotic cells, preferably mammalian cells, a re well known in the art. They, preferably, comprise regulatory sequences ensuring initiation of transcription and, optionally. poly-A signals ensuring termination of transcription and stabilization of the transcript. Additional regulatory elements may include transcriptional as well as translational enhancers. Examples for regulatory elements permitting expression in eukaryotic host cells are the AOXI or GAU promoter in yeast or the SMVP-, CMV- EPS-, SV40-, or RSV-promoter (Rous sarcoma virus), CMV-enhancer. SV40-enhancer or a globin intron in mammalian and other animal cells. Moreover, inducible or cell type-specific expression control sequences may be comprised in a polynucleotide of the present invention. Inducible expression control sequences may comprise tet or lac operator sequences or sequences inducible by heat shock or other environmental factors. Suitable expression control sequences are well known in the art. Besides elements which are responsible for the initiation of transcription, such regulatory elements may also comprise transcription termination signals, such as the SV40-poly-A site or the tk-poly-A site, downstream of the polynucleotide.

In a preferred embodiment, the polynucleotide comprises, more preferably consists of, a nucleic acid sequence of any one of SEQ ID NOs:134 to 176, more preferably any one of SEQ ID NO: 137, 150, 156 to 160, and 169 to 176, or a nucleic acid sequence at least 80%, preferably at least 90%, more preferably at least 95%, even more preferably at least 98%, most preferably at least 99% identical to any of the aforesaid SEQ ID NOs. In a more preferred embodiment, the polynucleotide comprises, more preferably consists of, a nucleic acid sequence of any one of SEQ ID NOs:134 to 176, more preferably any one of SEQ ID NO: 137, 150, 156 to 160, and 169 to 176.

The “clustered regularly interspaced short palindromic repeats” or “CRISPR” systems arc known to the skilled person, as described herein above. As is understood by the skilled person, the CRISPR system requires a “guidcRNA” (“gRNA”) to confer sequence specificity to the Cas nuclease.

The terms “CRISPR-associated endonuclease” and “Cas nuclease”, as used herein, both equally relate to an endonuclease, preferably an endo-DNase or ondo-RNase, more preferably an endo-DNase. recognizing a gRNA as specified herein, which is, in principle, known in the art. Preferably, the Cas nuclease is a type II CRISPR endonuclease. Preferably, the Cas nuclease is a CRISPR endonuclease from Prevotella and Francisella endonuclease, i.e. a Cpf1 endonuclease. More preferably, the CRISPR endonuclease is a Cas9 endonuclease. Preferably, the Cas9 nuclease is a Cas9 endonuclease from Staphylococcus aureus or is a Cas9 endonuclease from Streptococcus pyogenes, more preferably is a Cas9 endonuclease from Streptococcus pyogenes. Preferably, the Cas nuclease has an amino acid sequence as shown in SEQ ID NO: 5, preferably encoded by a nucleic acid sequence as shown in SEQ ID NO: 6. The term “fragment of a Cas nuclease”, as used herein, relates to a polypeptide fragment of a Cas nuclease which by itself is not catalytically active as a nuclease, however can be reconstituted to form a catalytically active nuclease by contacting said fragment with a second fragment which by itself is not catalytically active as well. Thus, a fragment of a Cas nuclease, as referred to herein, is a reconstitutable fragment. Preferably, reconstitution of Cas activity is accomplished by fusion of two non-identical fragments of a Cas polypeptide to auxiliary peptide sequences mediating either binding between the two fragments or covalent fusion due to split intein-mediated trans-splicing. Also preferably included by the term “Cas nuclease” is a variant of a Cas nuclease which is not catalytically active as an endonuclease, but has the activity of sequence-specific binding to a target polynucleotide in the presence of a gRNA (binding-only variant).

The terms “anti-CRISPR polypeptide” and “Acr polypeptide” are known to the skilled person and relate equally to a polypeptide having the activity of inhibiting at least one Cas nuclease, preferably a Cas9 nuclease. Acr polypeptides and methods for their identification are known in the art e.g. from Pawluk et al. (2016), Rauch et al. (2017), and Hynes et al. (2017). The inhibitory activity of a polypeptide to inhibit a Cas nuclease can be determined by determining the activity of said Cas nuclease in the presence of the suspected Acr polypeptide, preferably as specified herein in the Example of FIG. 2. Preferably, a polypeptide is identified as an Acr polypeptide if the activity of at least one Cas nuclease is inhibited significantly, preferably by at least 10%, more preferably by at least 20%, even more preferably by at least 30%, yet more preferably by at least 40%, most preferably by at least 50%, in an assay as specified above. More preferably, a polypeptide is identified as an Acr polypeptide if the activity of at least one Cas9 nuclease, more preferably of Cas9 endonuclease from Streptococcus pyogenes, is inhibited significantly, preferably by at least 10%, more preferably by at least 20%. even more preferably by at least 30%, yet more preferably by at least 40%, most preferably by at least 50%. in an assay as specified above. However, as will be understood by the skilled person, it is also within the capabilities of the skilled person to establish whether a polypeptide of interest is an Acr polypeptide for another Cas nuclease of interest. As will also be understood, preferably, the Acr polypeptide of the present invention is selected such that it inhibits the Cas nuclease intended to be used. Thus, e.g. in case the Cas9 endonuclease from Streptococcus pyogenes is intended to be used for genetic modification and shall be inhibited in at least a part of cells contacted therewith, an Acr polypeptide of a Listeria monocytogenes prophage or a Streptococcus thermophilus virulent phage may be used. Thus, preferably, the Acr polypeptide is an Acr polypeptide of a Listeria monocytogenes prophage, more preferably is an AcrIIA2 or AcrIIA4 polypeptide, most preferably an AcrIIA4 polypeptide. Preferably, the Acr polypeptide comprises, preferably consists of, an amino acid sequence as shown in SEQ ID NO: 1, more preferably encoded by a nucleic sequence comprising, preferably consisting of, the sequence as shown in SEQ ID NO: 2.

The term “stimulus” is used herein in a broad sense relating to any chemical or physical stimulus capable of acting on a cell and for which a receptor polypeptide is known. Thus, the stimulus may e.g. be radiation, in particular light, a chemical compound, a magnetic field, heat, cold, salinity, osmotic pressure, and the like. Preferably, the stimulus is light, preferably blue light. Also preferably, the stimulus is a chemical compound. Receptors for a variety of chemical compounds arc known in the art: preferred receptors tor chemical compounds are described herein below: in accordance, the stimulus preferably is a hormone, preferably estrogen; or is an antibiotic, preferably tetracycline or rapamycin. As will be understood, “reception” of a stimulus, preferably, is absorption of at least one photon in case the stimulus is light, and binding of the chemical compound to the receptor in case the stimulus is a chemical compound. The dosing of the stimulus will depend on the application, as is understood and as can be established by the skilled person. E.g. in case blue light is used as the stimulus in cell culture or on a body surface, an irradiance of from 0.1 W/m² to 25 W/m², preferably of from 0.5 W/m² to 10 W/m², more preferably of from 1 W/m² to 5 W/m² is preferred. Rapamycin, preferably, is used at a concentration of from 2 nM to 2 mM, more preferably of 10 nM to 1 mM, most preferably of from 20 nM to 500 nM.

The term “receptor domain”, is, in principle, understood by the skilled person to relate to a polypeptide or domain thereof reacting to a stimulus by changing conformation. Titus, the receptor domain preferably is a ligand-receptor domain or a sensor domain, more preferably a light receptor domain. Preferably, the conformational change induced by the stimulus causes relocalization of the fusion polypeptide to or from the nucleus of a host cell. Thus, preferably, a cognate stimulus, in particular binding of a cognate ligand to the receptor domain, causes the receptor domain, and. optionally, the fusion polypeptide comprising the same, present in the cytosol of a host cell to translocate into the nucleus, as is the case with e.g. the mammalian estrogen receptor. Also preferably, a cognate stimulus, in particular binding of a cognate ligand to the receptor domain, causes the receptor domain, and, optionally, the fusion polypeptide comprising the same, present in the nucleus of a host cell to translocate into the cytosol, as is the case with e.g. the LEXY domain as specified herein below and in the Examples. More preferably, the receptor domain is a conformational switch domain, i.e. preferably, the conformational change induced by the stimulus causes the distance between the N-terminus and the C-terminus of the receptor domain to decrease to at most 3 nm, more preferably at most 2.5 nm, still more preferably at most 2 nm, even more preferably at most 1.5 nm, most preferably at most 1 nm; or the conformational change induced by the stimulus causes the distance between the N-terminus and the C-terminus of the receptor domain to increase to at least 1.5 nm, more preferably at least 2 nm, still mote preferably at least 2.5 nm, most preferably at least 3 nm. Thus, preferably, the confonnational change induced by the stimulus causes the distance between the N-terminus and the C-terminus of the receptor domain to be of from 0.1 nm to 3 nm, preferably of from 0.2 nm to 2 nm, even more preferably of from 0.5 nm to 2 nm, still more preferably of from 0.75 to 1.5 nm, most preferably of about 1 nm; or the conformational change induced by the stimulus causes the distance between the N-terminus and the C-terminus of the receptor dot rut in to exceed of from 0.1 nm to 3 nm, preferably of from 0.2 nm to 2 nm, even more preferably of from 0.5 nm to 2 nm, still more preferably of from 0.75 to 1.5 nm, most preferably of about 1 nm. As will be understood, preferably, the receptor domain may be a conformational switch domain which is at the same time relocated to or from the nucleus upon binding a cognate ligand. In accordance with the above, the receptor domain preferably is selected from a light-oxygen-or-voltage (LOV) domain, a rapamycin binding domain, a phytochrome (Phy) domain, a cryptochrome (Cry) domain, a steroid receptor domain, and tetracycline binding domain. Preferably, the steroid receptor domain is an estrogen receptor domain, more preferably a ligand-binding domain of human estrogen receptor-o; also preferably, the tetracycline binding domain is a tetracycline domain of a tet repressor; also preferably, the rapamycin binding domain is an engineered FRB-iFKBP fusion domain, more preferably is a UniRapR domain as described in Dagliyan et al. (2015); also preferably, the LOV domain is a LOV2 domain, preferably from Arena saliva or Arabidopsis thaliana, more preferably from Avena sativa. Thus, more preferably, the receptor domain is a LOV domain or a rapamycin-binding domain, more preferably a LOV or a UniRapR domain. Also more preferably, the receptor domain is a LOV domain, most preferably a LOV2 domain; also more preferably, the receptor domain is a rapamycin-binding domain, most preferably a UniRapR domain. Preferably, the receptor domain comprises, preferably consists of, the amino acid sequence of SEQ ID NO: 34, preferably encoded by the nucleic acid sequence of SEQ ID NO: 35 or 120; also preferably, the receptor domain comprises, preferably consists of, the amino acid sequence of SEQ ID NO: 36, preferably encoded by the nucleic acid sequence of SEQ ID NO: 37.

The term “fusion polypeptide” is known to the skilled person to relate to a polypeptide wherein all components, i.e. in particular the Acr polypeptide and the receptor domain, are covalently linked and. preferably, are produced as a contiguous polypeptide chain. Thus, preferably, the fusion polypeptide of the present invention is expressed from a single gene, preferably a single open reading frame. Preferably, the fusion polypeptide comprises, more preferably consists of the amino acid sequence of one of SEQ ID NOs: 78 to 114, more preferably 88 to 107; preferably, said fusion polypeptide is encoded by a polynucleotide comprising, more preferably consisting of a nucleic acid sequence of SEQ I D NOs: 38 to 74, more preferably 48 to 67. The fusion polypeptide, preferably, has the activity of mediating stimulus-modulated inhibition of a Cas nuclease; thus, preferably, the fusion polypeptide mediates inhibition of a Cas nuclease in a host cell in the presence of a stimulus, but not in its absence; or mediates inhibition of a Cas nuclease in a host cell In the absence of a stimulus, but not in its presence. Thus, preferably, the fusion polypeptide has the activity of inhibiting a Cas nuclease and being relocated inside the cell in dependence of the presence of a stimulus; and/or, preferably, the fusion polypeptide has the activity of inhibiting a Cas nuclease in dependence of the presence or absence of a stimulus.

Preferably, the receptor domain is fused to the N-terminus of the Acr polypeptide in the fusion polypeptide. As used herein, the term “fused to the N-terminus of the Acr polypeptide” relates to being fused to one of the N-terminal amino acids of the Acr polypeptide, wherein the N-terminal amino acids are the first ten, preferably the first five amino acids; thus, the receptor domain may preferably be fused to the first, the second, the third, the fourth, or the fifth amino acid of the Acr polypeptide. As will be understood, the N-terminal amino acids of the Acr polypeptide preceding the fusion point may be included to the N-terminus of the receptor domain (i.e. the receptor domain may be inserted into the amino acid sequence of the Acr polypeptide close to the N-terminus), or, more preferably, they may be omitted (i.e. the receptor domain may be added at or close to the N-terminus of the amino acid sequence of the Acr polypeptide). Preferably, the receptor domain fused to the N-terminus of the Acr polypeptide is a LOV2 domain as specified elsewhere herein; in such case, preferably, the fusion polypeptide comprises an amino acid sequence selected from SEQ ID NOs: 23 to 28, more preferably comprises, preferably consists of, an amino acid sequence selected from SEQ ID NOs: 108 to 113, preferably encoded by a nucleic acid sequence selected from SEQ ID NOs: 68 to 73.

Also preferably, the receptor domain is fused to the C-terminus of the Acr polypeptide in the fusion polypeptide. As used herein, the term “fused to the C-terminus of the Acr polypeptide” relates to being fused to one of the C-terminal amino acids of the Acr polypeptide, wherein the C-terminal amino acids arc the last ten, preferably the last five amino acids; thus, the receptor domain may preferably be fused to the last, the penultimate, the third lust, the fourth last, or the fifth last amino acid of the Acr polypeptide. As will be understood, the C-terminal amino acids of the Acr polypeptide following the fusion point may be included to the C-terminus of the receptor domain (i.e. the receptor domain may be inserted into the amino acid sequence of the Acr polypeptide close to the C-terminus). or they may be omitted (i.e. the receptor domain may be added at or close to the C-terminus of the amino acid sequence of the Acr polypeptide). Preferably, the receptor domain fused to the C-terminus of the Acr polypeptide is a light-inducible nuclear export system domain (LEXY); in such case, preferably, the fusion polypeptide comprises, preferably consists of, the amino acid sequence of SEQ ID NO: 114, preferably encoded by SEQ ID NO: 74.

More preferably, the receptor domain is inserted into a surface-exposed bop of the Acr, preferably at an insertion site corresponding to one of amino acids 62 to 69 of an AcrIIA4 polypeptide, i.e. preferably, corresponding to one of amino acids 62 to 69 of SEQ ID NO: 1. As used herein, the expression “insertion site corresponding to amino acid X” relates to an insertion after amino acid X in the conventional N-terminus to C-terminus notation: tints, e.g. an insertion site corresponding to amino acid 63 relates to an insertion between amino acids 63 and 64. Still more preferably, the receptor domain is inserted into the Acr replacing at least one amino acid corresponding to one of amino acids 62 to 69 of the AcrIIA4 polypeptide. Preferably, at least one, two, three, four, or five amino acids are replaced, more preferably at least two amino acids are replaced. More preferably, at least one. two, three, or four amino acids corresponding to amino acids 64 to 67 of SEQ ID NO: 1 are replaced, more preferably at least two amino acids corresponding to amino acids 64 to 67 of SEQ ID NO: 1 are replaced. Even more preferably, the receptor domain is inserted into the Acr replacing one or two amino acids corresponding to amino acids 64 to 67 of SEQ ID NO: 1. Most preferably, the receptor domain is inserted into the Acr replacing the amino acid corresponding to amino acid 66 or replacing the amino acids corresponding to amino acids 65 and 66 of SEQ ID NO: 1.

Preferably, the fusion polypeptide comprises at least one linker peptide intervening the Acr polypeptide sequence and the receptor domain sequence at the fusion site. More preferably, in particular in case the receptor domain is inserted into a surface-exposed loop of the Acr, the fusion polypeptide comprises at least one linker peptide intervening the Acr polypeptide sequence and the receptor domain sequence at both fusion sites, i.e. at the transition from the Acr sequence to the receptor domain sequence and at the transition from the receptor domain sequence to the Acr sequence, wherein said linker sequences may be identical or may be different. Suitable linker sequences are known in the art. Preferably, the linker has a length of from 1 to 7 amino acids, more preferably, the linker consists of 2 or 3 amino acids comprising serine (S), glycine (G), alanine (A) and/or proline (P) residues, more preferably S and/or G residues. Preferably, said 1 to 7 amino acids comprised by said linker peptide are selected from the group consisting of serine (S), glycine (G), alanine (A) and proline (P). Preferred linker peptides are linker peptides comprising, preferably consisting of: the amino acid or amino acid sequence G, SG, SGG, GSG, GGSGGSG (SEQ ID NO: 32), or the inverse sequences GSGGSGG (SEQ ID NO: 33), GGS, or GS. More preferably, the linker is G, SG, SGG, or GSG for insertion at the junction Acr sequence to receptor domain sequence, and is GSG, GGS, GS, or G at the junction receptor domain sequence to Acr sequence. Preferably, the fusion polypeptide comprises one of SEQ ID NOs: 7 to 22 as a sequence into which the receptor domain is inserted.

Preferably, the fusion polypeptide comprises further domains and or peptides, e.g. preferably monitoring peptides and/or tags as specified herein below. Preferably, the fusion polypeptide further comprises a nuclear localization sequence (NLS), or a nuclear export sequence (NES). Preferably, the NLS is an SV40 NLS, a cMyc NLS, a nucleoplasmin NLS or a variant thereof (e.g. a cMyc^{PIA} NLS), which are known in the art. More preferably, the NLS is a SV40 NLS, a cMyc NLS. or a nucleoplasmin NLS. As will be understood, the NLS or NES is preferably included to improve potential leakiness of the fusion polypeptide and. accordingly, the decision whether to include an NLS or an NES will depend on the cellular localization of the fusion polypeptide lacking the additional element. Thus, in case e.g. the receptor domain is an estrogen receptor domain, which is located outside the nucleus in the absence of estrogen, preferably an NES would be included. In contrast, for constructs in winch modulation is not mediated by cellular re localization, an NLS is preferably included, e.g. preferably to ensure that the fusion polypeptide is located in the nucleus. Preferably, the fusion polypeptide comprises more than one receptor domain. More preferably, at least one of the further domains us essentially identical to the first receptor domain comprised in the fusion polypeptide; e.g., preferably, one receptor domain may be fused to the N-terminus of the Acr polypeptide, and an essentially identical receptor domain may be fused to the C-terminus of the Acr polypeptide. It is, however, also envisaged that the fusion polypeptide comprises two different receptor domains, e.g. preferably one mediating re localization and a second one being a conformational switch.

The terms “polypeptide” and “fusion polypeptide”, as used herein, preferably encompass variants of said polypeptides and fusion polypeptides, the terms “polypeptide variant” and “fusion polypeptide variant” relating to any chemical molecule comprising at least one polypeptide or fusion polypeptide as specified elsewhere herein, having the indicated activity, bur differing in primary structure from said polypeptide or fusion polypeptide indicated above. Thus, the polypeptide variant, preferably, is a mutein having the indicated activity. Preferably, the polypeptide variant comprises a peptide having an amino acid sequence corresponding to an amino acid sequence of 20 to 1000, more preferably 50 to 500, even more preferably 100 to 250 consecutive amino acids comprised in a polypeptide as specified above. Moreover, also encompassed arc further (fusion) polypeptide variants of the aforementioned polypeptides. Such (fusion) polypeptide variants have at least essentially the same biological activity as the specific polypeptides. Moreover, it is to be understood that a (fusion) polypeptide variant as referred to in accordance with the present invention shall have an amino acid sequence which differs due to at least one amino acid substitution, deletion and/or addition, wherein the amino acid sequence of the variant is still, preferably, at least 50%, 60%, 70%, 80%, 85%, 90%, 92%, 95%, 97%, 98%, or 99% identical with the amino acid sequence of the specific (fusion) polypeptide. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the sequence it is compared to for optimal alignment. The percentage is calculated by determining, preferably over the whole length of the polypeptide, the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search tor similarity method of Pearson and Lipman (1988), by computerized implementations of these algorithms (GAP, BHSTFIT, BLAST, PASTA, and TFASTA in tire Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and. thus, the degree of identity. Preferably, the default values of 5.00 for gap weight and 0.30 for gap weight length are used. (Fusion) polypeptide variants referred to herein may be allelic variants or any other species specific homologs, paralogs, or orthologs. Moreover, the (fusion) polypeptide variants referred to herein include fragments of the specific polypeptides or the aforementioned types of (fusion) polypeptide variants as long as these fragments and/or variants have the biological activity as referred to above. Such fragments may be or be derived from, e.g., degradation products or splice variants of the polypeptides, further included are variants which differ due to postranslational modifications such as phosphorylation, glycosylation, ubiquitinylation, sumoylation, or myristylation, by including non-natural amino acids, and/or by being peptidomimetics. Preferably, variants of the fusion polypeptide include circularly permutated variants of the fusion polypeptide in which e.g. at least one N- and/or C-terminal domain was shifted to a different position within the fusion polypeptide, e.g. based on the secondary structure of the Acr polypeptide as shown in the Examples.

Advantageously, it was found in the work underlying the present invention that a Cas nuclease can be conditionally inhibited by the fusion polypeptides proposed herein. Surprisingly, it was found that also inserting a conformational switch polypeptide into an Acr polypeptide enables conditional Cas inhibition. Underlying this finding is the identification of a surface-exposed loop of the Acr allowing insertion of additional domains without losing Cas-inhibitory activity.

The definitions made above apply mutatis mutandis to the following. Additional definitions and explanations made further below also apply for all embodiments described in this specification mutatis mutandis.

The present invention further relates to a vector comprising the polynucleotide according to the present invention.

As used herein, the term “vector” relates to a polynucleotide comprising structural determinants required for delivering into and/or stably maintaining and/or propagating the polynucleotide of the present invention in a cell said structural determinants optionally including the elements of an outer shell of a self-propagating entity, e.g. a virus. The term, preferably, encompasses phage, plasmid, and viral vectors as well as artificial chromosomes, such as bacterial or yeast artificial chromosomes. Thus, the vector may be or comprise RNA or DNA. Moreover, the term also relates to targeting constructs which allow for random or site-directed integration of the targeting construct into genomic DNA. Such target constructs, preferably, comprise DNA of sufficient length for either homologous or heterologous recombination as described in detail below. Hie vector encompassing the polynucleotide of the present invention, preferably, further comprises selectable markers for propagation and/or selection in a host. The vector may be delivered into a host cell by various techniques well known in the art. For example, a plasmid vector can he introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Retroviral vectors may be replication-competent or replication-defective. In the latter case, viral propagation generally will occur only in complementing host/cells. Preferably, the vector is an adeno-associated virus, preferably a replication-incompetent adeno-associated virus. Targeted delivery, i.e. delivery of a polynucleotide or vector into one or more cell population(s) or tissue(s) with high specificity may be achieved by viral vectors, which may have a natural tropism for edits) and/or tissue(s) of interest or may be retargeted thereto: however, also non-viral targeting methods are known to the skilled person, e.g. from Harris et al. (2010), Biomaterials 31(5): 998.

Preferably, in the vector of the invention the polynucleotide is operatively linked to expression control sequences as specified herein above. Thus, preferably, the vector is an expression vector. In this context, suitable expression vectors are known in the art such as Okayama-Berg cDNA expression vector pcDVI (Pharmacia), pBlucscript (Stratagene), pCDM8, pRc/CMV, pcDNA1, pcDNA3 (ThermoFisher) or pSPORT1 (Invitrogen). Analogous expression control vectors are also known for RNA vectors such as retroviruses. Preferably, the vector is an expression vector and a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which arc well known to those skilled in the art from standard text books can be used to construct recombinant viral vectors.

The present invention also relates to a bipartite anti-CRISPR (Acr) polypeptide comprising a first partial Acr polypeptide comprising amino acids corresponding to amino acids 10 to 62 of SEQ ID NO: 1, and a second partial Acr polypeptide comprising amino acids corresponding to amino acids 67 to 77 of SEQ ID NO: 1.

The term “bipartite polypeptide”, as used herein, relates to a polypeptide consisting of two partial polypeptides, having the activity of the polypeptide indicated: thus. the bipartite Acr polypeptide has the activity of inhibiting a Cas nuclease as specified above. Preferably, the bipartite Acr polypeptide is a non-naturally occurring polypeptide. The first partial Acr polypeptide comprises, preferably consists of, amino acids corresponding to amino acids 10 to 62 of SEQ ID NO: 1, preferably amino acids 5 to 64 of SEQ ID NO: 1, more preferably amino acids 1 to 64 of SEQ ID NO: 2 or a sequence at least 70% identical thereto. Preferably, the first partial Acr polypeptide comprises, preferably consists of, amino acids 10 to 62 of SEQ ID NO: 1, more preferably amino acids 5 to 64 of SEQ ID NO: 1, most preferably amino acids 1 to 64, of SEQ ID NO: 1. The second partial Acr polypeptide comprises, preferably consists of, amino acids corresponding to amino acids 69 to 77 of SEQ ID NO: 1, preferably amino acids 67 to 82 of SEQ ID NO: 1, more preferably amino acids 67 to 87 of SEQ ID NO: 1 or a sequence at least 70% identical thereto. Preferably, the second partial Acr polypeptide comprises, preferably consists of, amino acids 69 to 77 of SEQ ID NO: 1, more preferably amino acids 67 to 82 of SEQ ID NO:1, most preferably amino acids 67 to 87 of SEQ ID NO:1.

Preferably, the two partial polypeptides are covalently connected by insertion of at least 5, more preferably at least 8, more preferably at least 10, most preferably at least 25 amino acids between the two partial peptides. More preferably, the two partial polypeptides are covalently connected by insertion of a receptor domain as specified herein above. Thus, preferably, the bipartite Acr polypeptide is a fusion polypeptide of the present invention comprising a receptor domain inserted into the Acr at an insert ion she corresponding to one of amino acids 62 to 69 of an AcrIIA4 polypeptide (SEQ ID NO:1) as specified herein above. Also preferably, the two partial polypeptides are not covalently connected; tints, preferably, the two partial polypeptides are exclusively connected by at least one of ionic interactions, van der Waals interactions, and hydrophobic interactions. Preferably, the first and second partial Acr polypeptide are separately fused to the components of a receptor/ligand pair, e.g., preferably, the first partial polypeptide may be fused to biotin or a strep tag. and the second partial polypeptide may be fused to a streptavidin or a strep-tactin.

The present invention further relates to a fusion polypeptide encoded by a polynucleotide according to the present invention. In a preferred embodiment, the polypeptide is encoded by a nucleic acid sequence comprising, more preferably consisting of. any one of SEQ ID NOs:134 to 176, more preferably any one of SEQ ID NO: 137, 150, 156 to 160, and 160 to 176, or a nucleic acid sequence at least 80%, preferably at least 90%, more preferably at least 95%, even more preferably at least 98%, most preferably at least 99% identical to any of the aforesaid SEQ ID NOs. In a more preferred embodiment, the polypeptide is encoded by a nucleic acid sequence comprising, more preferably consisting of, a nucleic acid sequence of any one of SEQ ID NOs: 134 to 176, more preferably anyone of SEQ ID NO: 137, 150, 156 to 160, and 169 to 176.

Also, the present invention relates to a host cell comprising the polynucleotide according to the present invention, the vector according to the present invention, and/or the polypeptide according to the present invention.

As used herein, the term “host cell” relates to any cell capable of receiving, and optionally maintaining and/or propagating, the polynucleotide and/or the vector and/or the (fusion or bipartite) polypeptide of the present invention. Preferably, the cell is a bacterial cell, more preferably a cell of a common laboratory bacterial strain known in the art, most preferably an Escherichia strain, in particular an E. coli strain. Also preferably, the host cell is a eukaryotic cell, preferably a plant or yeast cell e.g. a cell of a strain of baker's yeast, or is an animal cell. More preferably, the host cell is an insect cell or a mammalian cell, in particular a mouse or rat cell. Even more preferably, the host cell is a mammalian cell, most preferably is a human cell.

Furthermore, the present invention relates to a polynucleotide according to the present invention, a vector according to the present invention, a polypeptide according to the present invention and/or a host cell according to the present invention for use in medicine and/or for use in treatment and/or prevention of genetic disease, neurodegenerative disease, cancer, and/or infectious disease.

The means and methods of the present invention are, in principle, usable in treatment and/or prevention of each and every disease for which genetic or epigenetic modification of a cell, preferably a specific type of cell is considered beneficial. Such is the case in particular in genetic disease, neurodegenerative disease, cancer, and infectious disease. As used herein, the term “genetic modification”, preferably, includes modification of any kind of nucleic acid comprised in a host cell at a given time, including nuclear DNA, organelle DNA (mitochondrial DNA or plastid DNA). but also nucleic acid from an infectious agent, either as free nucleic acid or covalently connected to the DNA of the host cell. Preferably, genetic modification is modification of nucleic acid, preferably DNA, present in the nucleus of a host cell.

The term “treatment” refers to an amelioration of the diseases or disorders referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the an using various well known statistic evaluation tools, e.g.. determination of confidence intervals, p-value determination. Student's i-test, Mann-Whitney test etc. Preferred confidence intervals arc at least 90%. at least 95%, at least 97%, at least 98% or at least 99 %. The p-values are, preferably, 0.1, 0.05, 0.01, 0.005, or 0.001. Preferably, the treatment shall be effective for at least 10%, at least 20%, at least 50%, at least 60%, at least 70%, at least 80%, or at least 90% of the subjects of a given cohort or population.

The term “preventing” refers to retaining health with respect to the diseases or disorders referred to herein for a certain period of time in a subject. It will be understood that said period of time is dependent on a variety of individual factors of the subject and the specific preventive treatment. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification.

The term “genetic disease”, as used herein, relates to a disease causally linked to one or more modifications, preferably mutations in the genome of an individual. Thus, preferably, the genetic disease is causally linked to one or more epigenetic changes, more preferably is causally linked to one or more genetic mutations. As will be understood, symptoms of a genetic disease often arc caused by expression of a mutated gene and/or lack of expression of a gene providing normal function of the gene product in one or more specific tissue(s) and/or cell type(s). Thus, it may be preferable to genetically modify by Cas activity only those cells in which the mutation contributes to disease. Preferably, the genetic disease is Duchenne muscular dystrophy, Huntington's disease. Hemophilia A/B, cystic fibrosis, myotubular myopathy, a glycogen storage disorder, or sickle cell anemia, the causes and symptoms of which are known to the skilled person from textbooks of medicine.

The term “neurodegenerative disease” relates to a disease caused by progressive loss of structure and/or function of neurons in the peripheral and/or central nervous system of an individual Preferably, the neurodegenerative disease is a neurodegenerative disease of motoneurons and/or a neurodegenerative disease of the central nervous system. Preferably, the neurodegenerative disease is Alzheimer's disease, amyotrophic lateral sclerosis (ALS), Parkinson's disease, or a spinocerebellar ataxia, preferably spinocerebellar ataxia type 1 (SCA1). As will be understood, many neurodegenerative diseases arc genetic diseases.

The term “cancer” is. in principle, understood by the skilled person and relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body. Preferably, also included by the term cancer is a relapse. Thus, preferably, the cancer is a non-solid cancer, e.g. a leukemia, or is a tumor of a solid cancer, a metastasis, or a relapse thereof, in particular is hepatocellular carcinoma, pancreatic cancer, osteosarcoma, leukemia or colorectal cancer. As is known to the skilled person, cancer cells accumulate mutations in particular in oncogenes or in tumor-suppressor genes, which may be amenable to correction by genetic modification. Moreover, the means and methods of the present invention may be used to induce cell death, e.g. via apoptosis, specifically in cancer cells. Preferably, treating cancer is reducing tumor and/or cancer cell burden in a subject. As will be understood by the skilled person, effectiveness of treatment of e.g. cancer is dependent on a variety of factors including, e.g. cancer stage and cancer type.

The term “infectious disease” is, in principle, understood by the skilled person. Preferably, the term as used herein relates to an infectious disease in which the replicative cycle of the infectious agent comprises at least one stage in which the genome of the infectious agent is present in a permissive host cell. Thus the infectious disease, preferably, is a viral infection, preferably is immunodeficiency virus infection, herpes virus infection, papillomavirus infection, or hepatitis B virus infection.

The present invention further relates to a kit according to the present invention, a vector according to the present invention, a polypeptide according to the present invention and/or a host cell according to the present invention and an agent providing a Cas nuclease activity in a host cell.

The term “kit”, as used herein, refers to a collection of the aforementioned compounds, means or reagents of the present invention which may or may not be packaged together. The components of the kit may be comprised by separate vials (i.e. as a kit of separate parts) or provided in a single vial. Moreover, it is to be understood that the kit of the present invention, preferably, is to be used for practicing the methods referred to elsewhere herein, it is, in an embodiment, envisaged that all components arc provided in a ready-to-use manner for practicing the methods referred to above. Further, the kit, in an embodiment, contains instructions for carrying out said methods. The instructions can be provided by a user's manual in paper or electronic form. In addition, the manual may comprise instructions for interpreting the results obtained when carrying out the aforementioned methods using the kit of the present invention.

The kit of the present invention further comprises an agent providing Cas nuclease activity. The term “agent providing Cas nuclease activity” is understood by live skilled person and includes polynucleotides and vectors mediating expression of a Cas nuclease in a host cell, a Cas polypeptide, as well as a host cell releasing a Cas polypeptide or a polynucleotide mediating expression of a Cas nuclease. Preferably, the agent providing Cas nuclease activity is a polynucleotide or vectors mediating expression of a Cas nuclease in a host cell, wherein said Cas nuclease is a Cas nuclease as specified herein above.

Preferably, the kit comprises further components. Preferably, the kit further comprises a polynucleotide encoding at least one guide RNA (gRNA). Also preferably, the kit further comprises at least one delivery means for at least one component it comprises, the term “delivery means” relating to any means suitable to mediate entry of a polynucleotide, polypeptide, and/or host cell of the kit to enter the relevant site in the body of a subject. Preferably, the kit provides an agent providing an appropriate stimulus tor the receptor domain or an agent being the stimulus itself, e.g. rapamycin. Preferably, in case the kit comprises a host cell of the invention, the relevant site, preferably, is the blood stream, a tumor mass, or a body cavity. Preferably, in case the kit comprises a polynucleotide or a polypeptide of the invention, the relevant site preferably is the interior of a host cell. Suitable delivery means arc known in the an and include in particular transfection reagents, packaging means, and the like. Preferably, the polynucleotides of the present invention are pre-packaged in a delivery means, e.g. in viral particles.

The present invention further relates to a method of providing a host cell comprising a stimulus-modulatable activity of a CRISPR-associated (Cas) nuclease comprising

a) introducing into said host cell a Cas nuclease:

b) introducing into said host cell a fusion polypeptide comprising an Acr polypeptide and a receptor domain according to the present invention;

c) thereby, providing a host cell comprising a stimulus-modulatable activity of a Cas nuclease.

The method of the present invention, preferably, is an in vitro method. Moreover, it may comprise steps in addition to those explicitly mentioned above. For example, further steps may relate, e.g., to providing a host cell for step a), or incubating the host cell after step b). Moreover, one or more of said steps maybe performed by automated equipment.

As will be understood, the Cas nuclease and/or the fusion polypeptide may be introduced into a host cell as such, i.e. as (a) polypeptide(s). Preferably, introducing a Cas nuclease is introducing a polynucleotide and/or vector mediating expression of a Cas nuclease. Also preferably, introducing a fusion polypeptide comprising an Acr polypeptide and a receptor domain according to the present invention is introducing a polynucleotide according to the present invention and/or a vector according to the present invention into said host cell. Appropriate means and methods for introducing a polynucleotide or a vector into a cell are well-known in the art.

The present invention also relates to a host cell produced or producible by the method of providing a host cell comprising a stimulus-modulatable activity of a Cas nuclease.

The present invention also relates to a method for treating genetic disease, neurodegenerative disease, cancer, and/or Infectious disease in a subject suffering therefrom, said method comprising

a) contacting a host cell of said subject with a Cas nuclease and with a fusion polypeptide comprising an anti-CRISPR (Acr) polypeptide and a receptor domain according to the present invention;

b) optionally, providing a stimulus causing the receptor domain to change conformation; and

c) thereby, treating genetic disease, neurodegenerative disease, cancer, and/or infectious disease.

The method for treating a subject of the present invention, preferably, is an in vivo method. In an embodiment, at least some steps of the method may, however, also be applied in vitro. Moreover, the method may comprise steps in addition to those explicitly mentioned above. For example, further steps may relate, e.g., to providing a sample of host cells, preferably permissive host cells, for step a), or incubating said cells for an appropriate time in or after step b). Moreover, one or more of said steps may be performed by automated equipment. According to the present invention, the polynucleotide, the vector, the host cell, and/or the components of the kit are, preferably, administered to a sample of the subject comprising permissive host cells, e.g. a blood sample, and said sample or cells derived thereof are re-administered to said subject after they were genetically modified. More preferably, the polynucleotide, the vector, the host cell, and/or the components of the kit are administered to the subject directly, e.g. by intravenous injection or topical application.

Thus, preferably, the polynucleotide, the vector, the host cell, and or the components of the kit of the present invention are provided as a pharmaceutical composition. The term “pharmaceutical composition”, as used herein, comprises the compounds of the present invention and optionally one or wore pharmaceutically acceptable carrier. The compounds of the present invention can be formulated as pharmaceutically acceptable salts. Acceptable salts comprise acetate, methylester. HCl, sulfate, chloride and the like. The pharmaceutical compositions are, preferably, administered topically or systemically. Suitable routes of administration conventionally used for drug administration are oral, intravenous, or parenteral administration as well as inhalation. However, depending on the nature and mode of action of a compound, the pharmaceutical compositions may be administered by other routes as well. For example, polynucleotide compounds may be administered in a gene therapy approach by using viral vectors or viruses or liposomes, as specified herein above. Moreover, the compounds can be administered in combination with other drugs either in a common pharmaceutical composition or as separated pharmaceutical compositions wherein said separated pharmaceutical compositions may be provided in form of a kit of parts. The compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The pharmaceutical carrier employed may be, for example, either a solid, a gel or a liquid. Exemplary of solid carriers arc lactose, terra alba, sucrose, talc, gelatin, agar, pectin, acacia, magnesium stearate, stearic acid and the like. Exemplary of liquid carriers are phosphate-buffered saline solution, syrup, oil such as peanut oil and olive oil, water, emulsions, various types of wetting agents, sterile solutions and die like. Similarly, the carrier or diluent may include time delay material well known to the art, such as glyceryl mono-stearate or glyceryl distearate alone or with a wax. Said suitable carriers comprise those mentioned above and others well known in the art, sec, e.g., Remington's Pharmaceutical Sciences, Mack Publishing Company, Easton, Pa. The diluent(s) is/are selected so as not to affect the biological activity of the combination. Examples of such diluents are distilled water, physiological saline. Ringer's solutions, dextrose solution, and Hank's solution. In addition, the pharmaceutical composition or formulation may also include other carriers, adjuvants, or nontoxic, nontherapeutic, nonimmunogenic stabilizers and the like.

A therapeutically effective dose refers to an amount of the compounds to be used in a pharmaceutical composition of the present invention which prevents, ameliorates or treats the symptoms accompanying a disease or condition referred to in this specification. Therapeutic efficacy and toxicity of such compounds can be determined by standard pharmaceutical procedures in cell cultures or experimental animals, e.g.. ED50 (the dose therapeutically effective in 50% of the population) and LD50 (the dose lethal to 50% of the population). The dose ratio between therapeutic arid toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depends upon many factors, including the patient's size, body surface area, age. the particular compound to be administered, sex, time and route of administration, general health, and other drugs being administered concurrently. Progress can be monitored by periodic assessment. A typical dose can be, for example, in the range of 1 to 1000 μg; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. However, depending on the subject and the mode of administration, the quantity of substance administration may vary over a wide range to provide from about 0.01 mg per kg body mass to about 10 mg per kg body mass. In case a viral vector, in particular adeno-associated viral vector is administered, preferred doses are from 5×10¹¹, to 2×10¹³ viral particles or viral genomes/kg body weight; as will be understood, these exemplary doses may be modified depending, in addition to the factors described above, on additional factors like type of virus, target organ, and the like. Preferably, the dose of the stimulus is adjusted such that the Cas nuclease is inhibited in at least 25%, more preferably at least 50%, most preferably at least 75% of cells in which inhibition is intended. As will be understood by the skilled person, the stimulus can be adjusted to achieve a pre-determined probability for a cell to inhibit or not inhibit Cas nuclease; e.g. preferably, in a population of host cells, the dose of the stimulus may be adjusted such that a predetermined fraction of cells undergoes a Cas-mediated excision event over a predetermined time period. In case the Cas nuclease is a binding-only variant as specified herein above, preferably, by adjusting the dose of the stimulus, the degree of binding of the binding-only Cas nuclease to the target polynucleotide can be modulated.

The pharmaceutical compositions and formulations referred to herein are administered at least once in order to treat or ameliorate or prevent a disease or condition recited in this specification. However, the said pharmaceutical compositions may be administered more than one time, for example from one to four times daily up to a non-limited number of days.

Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a earner or the diluent, or enclosed or encapsulated in a capsule, sachet, cachet, paper or other suitable containers or vehicles. The resulting formulations are to be adopted to the mode of administration, i.e. in the forms of tablets, capsules, suppositories, solutions, suspensions or the like. Dosage recommendations shall be indicated in the presenters or users instructions in order to anticipate dose adjustments depending on the considered recipient. Furthermore, the present invention relates to a use of a polynucleotide according to the present invention, a vector according to the present invention, a polypeptide according to the present invention for conditionally activating a Cas nuclease, preferably in a host cell; and to a use of a polynucleotide according to the present invention, a vector according to the present invention, a polypeptide according to the present invention and or a host cell according to the present invention for the manufacture of a medicament, preferably for the manufacture of a medicament for treating and/for preventing genetic disease, neurodegenerative disease, cancer, and/or infectious disease.

Preferably, the present invent ton also relates to a method for providing a host cell having stimulus-modulatable gene expression, comprising a) introducing into said host cell a binding-only variant of a Cas nuclease, optionally fused to a polypeptide regulating gene expression; b) introducing into said host cell a fusion polypeptide comprising an Acr polypeptide and a receptor domain according to the present invention; c) thereby, providing a host cell having stimulus-modulatable gene expression.

The method for providing a host cell having stimulus-modulatable gene expression of the present invention, preferably, is an in vitro method. Moreover, it may comprise steps in addition to those explicitly mentioned above. For example, further steps may relate, e.g., to providing a host cell for step a), incubating the host cell after step b), and/or contacting said host cell with, a gRNA preceding, concomitantly to, or following step a). Moreover, one or more of said steps may be performed by automated equipment. Preferably, the present invention also relates to a method for modulating gene expression in a host cell in a stimulus-dependent manner, comprising the steps of the method for providing a host cell having stimulus-modulatable gene expression, and the further steps of contacting said host cell with a gRNA at least partially complementary to a gene of interest and of providing said stimulus to said cell.

Preferably, the gRNA is selected to mediate binding of the binding-only variant of a Cas nuclease in the promoter region of a gene of interest, preferably preventing transcription factors and or RNA polymerase from binding; as is understood by the skilled person, repression of gene expression is expected in such case. Also preferably, the gRNA is selected to mediate binding of the binding-only variant of a Cas nuclease in the promoter region, enhancer region, the coding region, or a region adjacent thereto and the binding-only variant of a Cas nuclease is fused to an activating polypeptide. The term “polypeptide regulating gene expression”, as used herein, preferably relates to a polypeptide or fragment thereof having the activity of modulating transcription from a gene, preferably lacking sequence-specific DNA-binding activity, more preferably lacking DNA-binding activity. Preferably, the polypeptide regulating gene expression is a polypeptide repressing transcription if bound in the vicinity of one of the aforesaid gene regions, i.e. is a repressor polypeptide or domain thereof. Appropriate transcriptional repressor polypeptides or domains thereof arc known to the skilled person. More preferably, the polypeptide regulating gene expression is a polypeptide activating transcription if bound in the vicinity of one of the aforesaid gene regions, i.e. is an activating polypeptide or domain thereof. Thus, the activating polypeptide preferably is an activating domain of a transcriptional activator, or is a polypeptide mediating epigenetic changes increasing transcription. Thus, preferably, the activating polypeptide is a catalytically active fragment of a histone demethylase or of a histone acctyltransferase, more preferably of a histone acetyltransferase. Most preferably, the activating polypeptide is a catalytically active fragment of a p300 histone acetyltransferase.

Preferably, the present invention further relates to a method tor providing a host cell enabling stimulus-modulatable labelling of a genomic sequence of interest, comprising a) introducing into said host cell a binding-only variant of a Cas nuclease fused to a detectable label; b) introducing into said host cell a fusion polypeptide comprising an Acr polypeptide and a receptor domain according to the present invention; c) thereby, providing a host cell enabling stimulus-modulatable labelling of a genomic sequence of interest.

The method for providing a host cell enabling stimulus-modulatable labelling of a genomic sequence of interest of the present invention, preferably, is an in vitro method. Moreover, it may comprise steps in addition to those explicitly mentioned above. For example, further steps may relate, e.g., to providing a host cell for step a), incubating the host cell after step b), and/or contacting said host cell with a gRNA preceding, concomitantly to, or following step a). Moreover, one or more of said steps may be performed by automated equipment. Preferably, the present invention further relates to a method of labelling a genomic sequence of interest in a cell, comprising the steps of the method for providing a host cell enabling stimulus-modulatable labelling of a genomic sequence of interest, and the further steps of contacting said cell with a gRNA at least in pan complementary to said genomic sequence of interest, and providing said stimulus to said host cell.

Preferably, the “detectable label” is a label detectable by optical means, which are in principle known in the art. More preferably, the detectable label is an optically detectable polypeptide, more preferably a fluorescent polypeptide, even more preferably a green fluorescent protein (GFP) or a variant thereof, in particular a GFP, a yellow fluorescent protein (YFP). a blue fluorescent protein (BFP), or a red fluorescent protein (RFP), most preferably an RFP. As will be understood by the skilled person, the sequence to be labelled (sequence of interest) may be defined by selecting and cotransfecting an appropriate gRNA. As will be also understood, a host cell enabling stimulus-modulatable labelling of a genomic sequence of interest may be produced as specified, i.e. without contacting said host cell with a gRNA, and a gRNA may be introduced into the host cell at a later point in time.

In view of the above, the following embodiments arc particularly envisaged:

1. A polynucleotide encoding a fusion polypeptide comprising an anti-CRISPR (Acr) polypeptide, wherein said fusion polypeptide further comprises a receptor domain changing conformation upon reception of a stimulus.

2. The polynucleotide of embodiment 1, wherein said fusion polypeptide mediates stimulus-modulated inhibition of a CRISPR-associated (Cas) nuclease in a host cell.

3. The polynucleotide of embodiment 1or 2, wherein said stimulus is light, preferably blue light, or wherein said stimulus is a chemical compound, preferably is rapamycin.

4. The polynucleotide of any one of embodiments 1 to 3, wherein said receptor domain is fused to one of the terminal amino acids of the Acr polypeptide and/or is inserted into the Acr at an insertion site corresponding to one of amino acids 62 to 69 of the AcrIIA4 polypeptide (SEQ ID NO: 1).

5. The polynucleotide of any one of embodiments 1 to 4, wherein said fusion polypeptide comprises

(i) a receptor domain inserted into the Acr at an insertion site corresponding to one of amino acids 62 to 69 of an AcrIIA4 polypeptide (SEQ ID NO:1);

(ii) a receptor domain fused to one of the N-terminal amino acids of the Acr polypeptide;

(iii) a receptor domain fused to one of the C-terminal amino acids of the Acr polypeptide, wherein said receptor domain is directly fused to a nuclear export sequence (NES); or

(iv) any combination of (i) to (iii).

6. The polynucleotide of any one of embodiments 1 to 5, wherein said receptor domain is fused directly to one of the terminal amino acids, preferably the N-terminal amino acids, of the Acr polypeptide.

7. The polynucleotide of any one of embodiments 1 to 6, wherein said fusion polypeptide comprises a receptor domain inserted into the Acr at an insertion site corresponding to one of amino acids 62 to 69 of the AcrIIA4 polypeptide (SEQ ID NO: 1).

8. The polynucleotide of any one of embodiments 1 to 7, wherein said fusion polypeptide comprises at least two receptor domains and wherein one of said receptor domains is comprised in a structure C-terminal amino acid of the Acr polypeptide—receptor domain—NES.

9. The polynucleotide of any one of embodiments 1 to 8, wherein said polypeptide further comprises at least one further receptor domain.

10. The polynucleotide of any one of claims 1 to 9, wherein said receptor domain is selected from a light-oxygen-or-voltage (LOV) domain, a rapamycin-binding domain, a phytochrome (Phy) domain, a cryptochrome (Cry) domain, a steroid receptor domain, and tetracycline binding domain, preferably is a LOV domain.

11. The polynucleotide of any one of embodiments 1 to 10, wherein said receptor domain is a LOV2 domain, preferably from Avena saliva or Arabidopsis thaliana, preferably from Arena saliva.

12. The polynucleotide of any one of embodiments 1 to 11, wherein said fusion polypeptide further comprises at least one nuclear localization sequence (NLS), preferably a SV40NXS or a cMyc NLS.

13. The polynucleotide of any one of embodiments 1 to 12, wherein said receptor domain comprises an amino acid sequence at least 70% identical to five amino acid sequence of SEQ ID NO: 34 or 36, preferably comprises the amino acid sequence of SEQ ID NO: 34 or 36.

14. The polynucleotide of any one of embodiments 1 to 13, wherein said Acr polypeptide is an AcrII polypeptide, preferably wherein the polynucleotide comprises, more preferably consists of, a nucleic acid sequence of any one of SEQ ID NOs: 134 to 176, more preferably any one of SEQ ID NO: 137, 150, 156 to 160, and 169 to 176.

15. The polynucleotide of any one of embodiments 1 to 14, wherein said Acr polypeptide is an AcrIIA4 polypeptide, preferably from listeria monocytogenes prophage.

16. The polynucleotide of any one of embodiments 1 to 15, wherein said Acr polypeptide comprises an amino acid sequence at least 70% identical to tin* amino acid sequence of SEQ ID NO: 1, preferably comprises the amino acid sequence of SEQ ID NO: 1.

17. The polynucleotide of any one of embodiments 1 to 16, wherein said fusion polypeptide comprises an amino acid sequence at least 70% identical to an amino acid sequence of SEQ ID NOs: 78 to 114, more preferably SEQ ID NOs 88 to 107, preferably comprises the amino acid sequence of one of SEQ ID NOs: 78 to 114, more preferably SEQ ID NOs: 88 to 107.

18. A vector comprising the polynucleotide according to any one of embodiments 1 to 17, preferably wherein said vector is an expression vector.

19. A bipartite anti-CRISPR (Acr) polypeptide comprising a first partial Acr polypeptide comprising amino acids corresponding to amino acids 10 to 62 of SEQ ID NO: 1, and a second partial Acr polypeptide comprising amino acids corresponding to amino acids 67 to 77 of SEQ ID NO: 1.

20. The bipartite Acr polypeptide of embodiment 19, wherein said first and second partial Acr polypeptide are comprised in the same fusion polypeptide.

21. The bipartite Acr polypeptide of embodiment 19 or 20, wherein said first and second partial Acr polypeptide are intervened by a receptor domain in said fusion polypeptide.

22. The bipartite Acr polypeptide of any one of embodiments 19 to 21, wherein in at least one conformation of said bipartite Acr polypeptide the C-terminal amino acid of the first partial Acr polypeptide is less than 3 nm from the N-terminus of the second partial Acr polypeptide.

23. The bipartite Acr polypeptide of embodiment 19, wherein said first and second partial Acr polypeptide are separately fused to the components of a receptor/ligand pair.

24. The bipartite Acr polypeptide of any one of embodiments 19 to 22, wherein said bipartite Acr polypeptide is a polypeptide encoded by a polynucleotide according to any one of embodiments 1 to 17, preferably encoded by a nucleic acid sequence comprising, more preferably consisting of. a nucleic acid sequence of any one of SEQ ID NOs:134 to 176, more preferably any one of SEQ ID NO: 137, 150, 156 to 160, and 169 to 176.

25. A fusion polypeptide encoded by a polynucleotide according to any one of embodiments 1 to 17.

26. A host cell comprising the polynucleotide according to any one of embodiments 1 to 17, the vector according to embodiment 18, and or the polypeptide according to any one of embodiments 19 to 25.

27. A polynucleotide according to any one of embodiments 1 to 17, a vector according to embodiment 18, a polypeptide according to any one of embodiments 19 to 25, and/or a host cell according to embodiment 26 for use in medicine.

28. A polynucleotide according to any one of embodiments 1 to 17, a vector according to embodiment 18, a polypeptide according to any one of embodiments 19 to 25, and/or a host cell according to embodiment 26 for use in treatment and/or prevention of genetic disease, neurodegenerative disease, cancer, and/or infectious disease.

29. The polynucleotide, vector, polypeptide, and/or host cell for use according to embodiment 22, wherein said genetic disease is Duchenne muscular dystrophy, Huntington's disease. Hemophilia A/B, cystic fibrosis, myotubular myopathy, a glycogen storage disorder, or sickle cell anemia; wherein said neurodegenerative disease is Alzheimer's disease, amyotrophic lateral sclerosis (ALS), Parkinson's disease, or spinocerebellar ataxia type 1 (SCA1); wherein said cancer is hepatocellular carcinoma, pancreatic cancer, osteosarcoma, leukemia or colorectal cancer; and/or wherein said infectious disease is human immunodeficiency virus infection, herpes virus infection, papillomavirus infection, or hepatitis B virus infection.

30. A kit comprising the polynucleotide according to any one of embodiments 1 to 17, a vector according to embodiment 18, a polypeptide according to any one of embodiments 19 to 25, and/or a host cell according to embodiment 26 and an agent providing a Cas nuclease activity in a host cell.

31. The kit of embodiment 30, wherein said Cas nuclease activity is provided by a Cas polypeptide and/or a polynucleotide encoding a Cas polypeptide.

32. A method of providing a host cell comprising a stimulus-modulatable activity of a CRISPR-associated (Cas) nuclease comprising

a) introducing into said host cell a Cas nuclease;

b) introducing into said host cell a fusion polypeptide comprising an Acr polypeptide and a receptor domain according to embodiment 26;

c) thereby, providing a host cell comprising a stimulus-modulatable activity of a Cas nuclease.

33. The method of embodiment 32, wherein introducing into said host cell a Cas nuclease is contacting said host cell with a polynucleotide comprising an expressible sequence encoding said Cas nuclease.

34. The method of embodiment 32 or 33, wherein introducing into said host cell a fusion polypeptide comprising an Acr polypeptide and a receptor domain is contacting .said host cell with a polynucleotide comprising an expressible gene encoding said fusion polypeptide comprising an Acr polypeptide and a receptor domain, preferably is contacting said host cell with a polynucleotide according to any one of embodiments 1 to 17.

35. A host cell produced or producible by the method according to any one of embodiments 32 to 34.

36. A method for treating genetic disease, neurodegenerative disease, cancer, and/or infectious disease in a subject suffering therefrom, said method comprising

a) contacting a host cell of said subject with a Cas nuclease and with a fusion polypeptide comprising an anti-CRISPR (Acr) polypeptide and a receptor domain according to embodiment 25;

b) optionally, providing a stimulus causing the receptor domain to change conformation; and

c) thereby, treating genetic disease, neurodegenerative disease, cancer, and/or infectious disease.

37. The method of embodiment 36, wherein said method comprises contacting at least a fraction of cells of said subject with said stimulus causing the receptor domain to change conformation,

38. The method of embodiment 36 or 37, wherein said method further comprises contacting said host cell with at least one gRNA.

39. The method of any one of embodiments 36 to 38, wherein contacting a host cell with a gRNA is contacting said host cell with a polynucleotide comprising an expressible gene encoding said gRNA.

40. The method of any one of embodiments 36 to 39, wherein contacting a host cell with a Cas nuclease is contacting said host cell with a polynucleotide comprising an expressible gene encoding said Cas nuclease.

41. The method of any one of embodiments 36 to 40, wherein contacting a host cell with a fusion polypeptide comprising an Acr polypeptide and a receptor domain is contacting said host cell with a polynucleotide comprising an expressible gene encoding said fusion polypeptide comprising an Acr polypeptide and a receptor domain, preferably is contacting said host cell with a polynucleotide according to any one of embodiments 1 to 17.

42. Use of a polynucleotide according to any one of embodiments 1 to 17, a vector according to embodiment 18, and/or a polypeptide according to embodiment 26 for conditionally activating a Cas nuclease, preferably in a host cell.

43. Use of a polynucleotide according to any one of embodiments 1 to 17, a vector according to embodiment 18, a polypeptide according to any one of embodiments 19 to 25, and/or a host cell according to embodiment 26 for the manufacture of a medicament, preferably for the manufacture of a medicament for treating and/or preventing genetic disease, neurodegenerative disease, cancer, and/or infectious disease.

44. Method for providing a host cell having stimulus-modulatable gene expression, comprising a) introducing into said host cell a binding-only variant of a Cas nuclease, optionally fused to a polypeptide regulating gene expression; b) introducing into said host cell a fusion polypeptide comprising an Acr polypeptide and a receptor domain according to the present invention; c) thereby, providing a host cell having stimulus-modulatable gene expression.

45. Method for modulating gene expression in a host cell in a stimulus-dependent manner, comprising the steps of the method of embodiment 44 and the further steps of contacting said host cell with a gRNA at least partially complementary to a gene of interest and of providing said stimulus to said cell.

46. Method for providing a host cell enabling stimulus-modulatable labelling of a genomic sequence of interest, comprising a) introducing into said host cell a binding-only variant of a Cas nuclease fused to a detectable label; b) introducing into said host cell a fusion polypeptide comprising an Acr polypeptide and a receptor domain according to the present invention; c) thereby, providing a host cell enabling stimulus-modulatable labelling of a genomic sequence of interest.

47. Method of labelling a genomic sequence of interest in a cell comprising the steps of the method of embodiment 46 and the further steps of contacting said cell with a gRNA at least in part complementary to said genomic sequence of interest, and providing said stimulus to said host cell.

All references cited in this specification are herewith incorporated by reference with respect to their entire disclosure content, and the disclosure content specifically mentioned in this specification.

### FIGURE LEGENDS

FIG. 1: Light-induced disorder enables tight control of AcrIIA4. (A) Schematic of LOV2 insertion library generation. The LOV2 domain was inserted at 41 different positions into AcrIIA4 (indicated in C). Light-induced unfolding of the LOV2 domain should then cause disorder of the AcrIIA4 structure and hence inhibition of AcrIIA4, i.e. release of Cas9 activity (see FIG. 1B). (B) LOV2-AcrIIA4 enables light-dependent genome editing. The LOV2-AcrIIA4 hybrid indicated with * in (C) was co-transfectcd into HEK 293T alongside a Cas9 expression vector and a CFTR locus-targeting guideRNA. Sixteen h post transfection, cells were irradiated with blue light (7 s ON. 7 s OFF, 3 W/m²) for 32 h or kept in the dark as control. A T7 endonuclease assay was performed to monitor target locus cleavage. (Q Screen of LOV2-AcrIIA4 hybrid constructs. The LOV2 domain was inserted at the indicated positions into AcrIIA4 sequence (SEQ ID NO: 1). primarily into loops (bottom; Sec.Strct., secondary structure, derived from Dong et al., 2017). AcrIIA4-LOV2 hybrid variants were then investigated for their ability to inhibit SpyCas9 in the absence of blue light (bar chart, top). HEK 293T cells were transfected with the constructs in FIG. 6A alongside the indicated AcrIIA4-LOV2 hybrid. Forty-eight h post-transfection, a dual luciferase assay was performed. Firefly photon counts were normalized to Renilla photon counts. One loop comprising amino acids 62 to 69 tolerates the LOV2 insertion. Data represent means±s.e.m., 3 replicates, polypeptides with insertions at positions between amino acids 62 and 69 are those of SEQ ID NOs: 78 to 84 (encoded by SEQ ID NOs: 38 to 44, respectively), insertion between amino acids 85 to 87 or appended to amino acid 87 are those of SEQ ID NOs: 85 to 87 (encoded by SEQ ID NOs: 45 to 47, respectively).

FIG. 2: Improved light-induced disorder by systematic linker variation and embedding into the target protein. (A) Second generation LOV2-AcrIIA4 hybrids based on the variant indicated with * in FIG. 1C. Variants differ by the presence or absence of short OS linkers (bold) as well as optional deletion of AcrIIA4 residue E66 or Q65/E66; fusion site sequences are those of SEQ ID NOs: 7 to 15; fusion polypeptide sequences are those of SEQ ID NOs: 88 to 96 (encoded by SEQ ID NOs: 48 to 56). (B) Screen of the second generation LOV2-AcrIIA4 hybrid variants (numbers correspond to constructs in A). HEK 293T cells were transfected with the constructs in FIG. 6A alongside the indicated AcrIIA4-LOV2 hybrid. Six h after transfection, cells were irradiated with blue light (5 s ON, 15 s OFF, 2 W/m2) or kept in the dark for 42 h. A dual luciferase assay was performed. Firefly photon counts were normalized to Renilla photon counts. Data represent means from 2 replicates. (C) Third generation LOV2-AcrIIA4 hybrids with elongated linkers. (B, D) Acr, Res. #, AcrIIA4 residue number; fusion site sequences are those of SEQ ID NOs: 7, 15, and 16 to 19, fusion polypeptide #10 to 13 sequences are those of SEQ ID NOs: 97 to 100 (encoded by SEQ ID NOs: 57 to 60). (D) Screen of the third generation L0V2-AcrIIA4 hybrid variants by luciferase assay (numbers correspond to constructs in A and C). Experiment was performed as in B. Data represent means from at least 3 replicates. (E) LOV2-AcrIIA4 enables light-dependent genome editing. Indicated LOV2-AcrIIA4 hybrids from B and D were co-transfected into HEK 2931 alongside a Cas9 expression vector and a CFTR locus-targeting guideRNA. Six h post transfection, cells were irradiated with blue light (5 s ON, 10 s OFF, 3 W/m2) for 42 h or kept in the dark as control. A T7 endonuclease assay was performed to monitor target locus cleavage.

FIG. 3: Rapamycin control of AcrIIA4. (A) Schematics of rapamycin-inducible AcrIIA4 variants bearing a UniRapR domain inserted into the identified engineering hotspot. Variants differ by the presence or absence of short OS linkers (bold) as well as optional deletion of AcrIIA4 residue E66 or Q65/E66; fusion site sequences are those of SEQ ID NOs: 1 (#1), 10 (#2), 13 (#3), 15 (#4), 21 to 22 (#5 to #6), and 17 (#7); fusion polypeptide sequences are those of SEQ ID NOs; 101 to 107 (encoded by SEQ ID NOs: 61 to 67). (B) Screen of UniRapR-AcrIIA4 hybrids (numbers correspond to constructs in A). HEK 293Y cells were transfected with the constructs in FIG. 6A alongside the indicated UniRapR-AcrIIA4 hybrid in A. Six h post transfection, cells were treated with 10 μM rapamycin for 42 h or DMSO (rapamycin solvent) as control. A dual luciferase assay was performed. Firefly photon counts were normalized to Renilla photon counts and resulting values were normalized to the corresponding positive controls (reporter+gRNA). Data represent means from 2 replicates.

FIG. 4: N-terminal LOV2 fusion also enables negative AcrIIA4 regulation with light. (A) Schematic of used constructs and partial sequences of the tested LOV2-Jα-AcrIIA4 hybrids. The LOV2-Jα part is underlined. Variant #8 is a fusion of the wild type LOV2 (L404-L546) to wild type AcrIIA4 (without the AcrIIA4 methionine-encoding start codon). All other variants bear mutations or insertions (indicated in bold), or deletions introduced to alter the Jα hydrophobicity or its possible interaction with the AcrIIA4 domain; fusion site sequences are those of SEQ ID NOs: 23 to 28; fusion polypeptide sequences are those of SEQ ID NOs: 108 to 113 (encoded by SEQ ID NOs: 68 to 73). (B) Bar plot showing dCas9-VP64 transactivation assay in HEK 293T cells transfected with the indicated vectors in A alongside a dCas9-VP64 vector (SEQ ID NO: 117), a TetO-dependent luciferase reporter (SEQ ID NO: 118), a TetO-targeting guideRNA as well as a constitutive Renilla expression vector for normalization purposes (refer to FIG. 6C). Cells were irradiated with blue light (3 s ON, 17 s OFF, 2 W/m²) for 42 h or kept in the dark as control. Subsequently, a dual luciferase assay was performed. Firefly photon counts were normalized to Renilla photon counts. Data represent means±s.e.m., 3 replicates. (C) Bar plot showing Cas9 reporter cleavage assay in HEK 293T cells transfected with the indicated vectors in FIG. 6A alongside the corresponding LOV2-Jα-AcrIIA4 hybrid in A. Cells were irradiated with blue light (3 s ON, 17 s OFF, 2 W/m2) for 42 h or kept in the dark, followed by a dual luciferase assay as in B. Data represent means±s.e.m., 3 replicates.

FIG. 5: Induced nuclear export inhibits AcrIIA4 function. (A) Schematic of AcrIIA4 construct fused to mCherry-LEXY. LEXY, light-inducible nuclear export system; fusion polypeptide sequence is SEQ ID NO: 114 (encoded by SEQ ID NO: 74). (B) Fluorescence images of HEK 293T cells transfected with the construct in A. mCherry images were taken prior to induction (Preinduction), after 15 min of irradiation with blue light (Post Activation) and after an additional 20 min recovery phase in the absence of blue light (Post Recovery). (C) Line profile of the indicated cell in B, (D) Bar plot showing control of dCas9-VP64-mediated luciferase reporter activation by NLS-AcrIIA4-mCherry-LEXY. HEK 293T cells were transfected with the constructs in FIG. 6C alongside a constitutive Renilla expression vector (for normalization purposes) and optionally a wildtype AcrIIA4 expression vector or the NLS-AcrIIA4-mCherry-LEXY vector in A. The used ratio of dCas9-VP64 and AcrIIA4-mCherry-LEXY-encoding vector during transfection was varied as indicated. Cells were irradiated with blue light (3 s ON, 17 s OFF, 2 W/m²) for 42 h or kept in the dark, followed by dual-luciferase assay. Firefly luciferase photon counts were normalized to Renilla photon counts, 1, reporter+guideRNA; 2-4, reporter+guide RNA+dCas9-VP64; 2, no AcrIIA4; 3, NLS-AcrIIA4-mCherry-LEXY; 4, wild type AcrIIA4.

FIG. 6: Targeting AcrIIA4 to the nucleus improves Cas9 inhibition. (A) Schematic of Cas9 reporter cleavage assay. Co-delivery of Cas9, a firefly luciferase reporter and a luciferase-targeting guideRNA results in potent luciferase knockdown. The firefly reporter also bears a constitutive Renilla expression cassette for normalization purposes (SEQ ID NO: 119). AcrIIA4-mediated Cas9 inhibition prevents reporter knockdown. (B) Cas9 reporter cleavage assay in HEK 293T cells co-transfected with the constructs in A alongside an AcrIIA4 expression vector bearing a SV40 NLS at the N-terminus or not; the N-terminal SV40 NLS fusion to AcrIIA4 had the amino acid sequence of SEQ ID NO: 115, encoded by the nucleic acid sequence of SEQ ID NO: 75. Forty-eight h post-transfection, firefly and Renilla luciferase activity were measured by dual-luciferase assay. Firefly luciferase photon counts were normalized to Renilla photon counts. 1, reporter+guideRNA: 2-4 reporter+guideRNA+Cas9; 2, no AcrII4; 3, wild-type AcrIIA4; 4, SV49 NLS-AcrIIA4. Data are means±s.e.m., 3 replicates. (C) Schematic of dCas9-VP64 reporter trans-activation assay. Co-delivery of dCas9-VP64 and a TetO-targeting guideRNA results in potent activation of a firefly luciferase reporter driven from a minimal promoter preceded by TetO repeats. (D) dCas9-VP64 reporter trans-activation assay in HEK 293T cells co-transfected with the constructs in C as well as a constitutive Renilla expression vector (for normalization) and an AcrIIA4 expression vector bearing a cMyc^{PIA} NLS at the N-terminus or not. Forty-eight h post -transfection, firefly and Renilla luciferase activity were measured by dual-luciferase assay. Firefly luciferase photon counts were normalized to Renilla photon counts. 1, reporter 4 guideRNA; 2-4 reporter+guideRNA+dCas9-VP64; 2, no AcrIIA4; 3, wild-type AcrIIA4; 4, cMyc^{PIA} NLS-mCherry-AcrIIA4. Data are means±s.e.m., 3 replicates; the N-terminal cMyc^{PIA} NLS fusion to AcrIIA4 had the amino acid sequence of SEQ ID NO: 116, encoded by the nucleic acid sequence of SEQ ID NO: 76.

FIG. 7: Schematic and sequences of further Acr-LOV hybrids. The Acr-LOV hybrids can e.g. be encoded by the nucleic acid sequences provided as SEQ ID NOs: 134 to 149.

FIG. 8: Eight control of luciferase reporter cleavage by different Act-LOV hybrids. HEK 293T cells expressing Cas9, a luciferase reporter (Rep), a reporter-targeting gRNA and the indicated LOV-Acr variant in FIG. 7 were irradiated with 3 W per m pulsatile blue light for 48 h or kept in the dark followed by luciferase assay. Data arc means±s.d.

FIG. 9: Cas9 inhibition is dose-dependent. HEK 293T cells were co-transfected with plasmids encoding (i) Acr-LOV hybrid, (ii) Cas9 and (iii) a luciferase reporter as well as a gRNA targeting the luciferase gene. The vector mass ratio of the transfected Cas9 and Acr-LOV construct was varied between 10:1 and 1:1, as indicated. Six hours post-transfection, cells were irradiated with pulsatile blue light (5 s ON, 10 s OFF; 2.5 W per m²) for 30 h or kept in the dark as control before assessing luciferase activity. Data are means±s.e.m.

FIG. 10: Light-dependent editing of endogenous loci in HEK 293T cells. Transgenes were delivered by transient plasmid DNA transfection or AAV transduction. Light-mediated indel mutation of human CCR5 (a), CFFR (b) or EMX1 (c) locus. Cells were co-transfected or transduced with vectors expressing CASANOVA, Cas9 and a locus-specific gRNA, and then exposed to blue light for 70 h or kept in the dark as control. For the transfection samples, the used vector mass ratio of the Acr:Cas9 construct is indicated. Editing frequencies wore evaluated by mismatch-sensitive T7 endonuclease assay. Representative gel images and corresponding quantifications of editing efficiencies are shown. Data arc means±s.e.m. Wt, wild-type. CN, CASANOVA. *P<0.05. **P<0.01, ***P<0.001 by Student's t-test. (g) Note that CCR5 T7 fragments have the same size.

FIG. 11: Acr-LOV hybrids carrying a C450A LOV2 pseudodark mutation arc still light-responsive. (a) Light-dependent luciferase reporter cleavage mediated by different Acr-LOV hybrid pseudodark mutants. HEK 293T cells were co-transfected with plasmids encoding (i) the indicated Acr-LOV hybrid variant, (ii) Cas9 and (iii) a luciferase reporter as well as a gRNA targeting the luciferase gene. Six hours post-transfection, cells were irradiated with pulsatile blue light for 48 h or kept in the dark as control before assessing luciferase activity. Data arc means±s.d., (b) Quantification of light-mediated indel mutation of the human CCR5 focus by T7 endonuclease assays. HEK 293T cells were co-transfected with constructs expressing Cas9, CCR5 locus-targeting gRNA and the indicated Acr-LOV hybrid variant and exposed to blue light for 470 h or kept in the dark as control. During transfection, the vector mass ratio of Acr-LOV:Cas9 construct was varied as indicated, n.d., not determined. The Acr-LOV hybrids can e.g. be encoded by the nucleic acid sequences provided as SEQ ID NOs:150 to 155.

FIG. 12: Cas9 inhibition can be modulated via mutations that affect docking of the LOV2 terminal helices, (a) Light-dependent luciferase reporter cleavage mediated by different Acr-LOV hybrid mutants. HEK 293T cells were co-transfected with plasmids encoding (i) the indicated Acr-LOV hybrid variant, (ii) Cas9 and (iii) a luciferase reporter as well as a gRNA targeting the luciferase gene. Six hours post-transfection, cells were irradiated with pulsatile blue light for 48 h or kept in the dark as control before assessing luciferase activity. Data arc means±s.d., (b) Quantification of light-mediated indel mutation of the human CCR5 locus by T7 endonuclease assay. HEK 293T cells were co-transfected with constructs expressing the Cas9, the CCR5 locus-targeting gRNA and the indicated Acr-LOV hybrid variant and exposed to blue light for 70 h or kept in the dark as control. During transfection, the vector mass ratio of Acr-LOV:Cas9 construct was varied as indicated. Data arc moans. The Acr-LOV hybrids can e.g. be encoded by the nucleic acid sequences provided as SEQ ID NOs:156 to 165.

FIG. 13; In silico docking analysis reveals Acr mutations that improve CASANOVA performance, (a) Light-dependent luciferase reporter cleavage mediated by different Acr-LOV hybrid mutants. HEK 293T cells were co-transfected with vectors encoding (i) the indicated Acr-LOV hybrid variant, (ii) Cas9 and (in) a luciferase reporter as well as a gRNA targeting the luciferase gene. Six hours post-transfection, cells were irradiated with pulsatile blue light for 48 h or kept in the dark as control before assessing the luciferase activity. Data arc means±s.d., (b) Quantification of light-mediated indel mutation of the human CCR5 locus by T7 endonuclease assay. HEK 293T cells were co-transfected with constructs expressing Cas9, the CCR5 locus-targeting gRNA and the indicated Acr-LOV hybrid variant and exposed to blue light for 70 h or kept in the dark as control. During transfection, the vector mass ratio of Acr-LOV:Cas9 construct was varied as indicated. n.d., not determined. The Acr-LOV hybrids can e.g. be encoded by the nucleic acid sequences provided as SEQ ID NOs:166 to 176.

FIG. 14: Comparison of light-dependent indel mutation by CASANOVA and its corresponding S46D and T16F mutants. HEK 293T cells were co-transfected with constructs expressing Cas9, a gRNA and the indicated CASANOVA variant and exposed to blue light for 70 h or kept in the dark as control. During transfection, the vector mass ratio of Acr-LOV:Cas9 construct was varied as indicated. Editing frequencies were evaluated by mismatch-sensitive T7 endonuclease assay, (a) Indel mutation of CCR5 locus and (b) indel mutation of EMX1 locus, (a-b) Data arc means±s.e.m.

FIG. 15: Optogenetic control of xCas9. Light-dependent luciferase reporter cleavage mediated by different Acr-LOV hybrid mutants. HEK 293T cells were co-transfected with vectors encoding (i) the indicated Acr-LOV hybrid variant, (ii) xCas9 and (iii) a luciferase reporter as well as a gRNA targeting the luciferase gene. Six hours post-transfection, cells were irradiated with pulsatile blue light for 48 h or kept in the dark as control before assessing luciferase activity. Data are mean±s.d.

FIG. 16: Optogenetic control of gene expression, (a) Schematics showing concept of light-mediated activation of IL1RN expression using CASANOVA and a dCas9-based acetyltransferase (dCas9-p300) targeted to the IL1RN promoter, (b) Light control of IL1RN expression. HEK 293T cells expressing CASANOVA and a dCas9-p300 fusion targeted to the IL1RN promoter via four gRNAs were exposed to blue light for 44 h or kept in the dark. IL1RN expression was assessed by quantitative RT-PCR. Data arc means±s.e.m.

FIG. 17: Schematics showing concept of optogenetic control of telomere labeling.

FIG. 18; Analysis of light-mediated telomere recruitment in fixed samples. U2OS cells expressing dCas9-3xRFP. a tetomere-targeting gRNA and CASANOVA for wild-type or no Acr instead of CASANOVA) were exposed to blue light for 20 h or kept in the dark as control. Telomere labeling in individual nuclei was then quantified by automated image analysis in KNIME. The violin plot shows the distribution, while black bars and grey dots indicate the median and individual data points, respectively. **P<2.2·10⁻¹⁶ by Wilcoxon rank-sum test.

The following Examples shall merely illustrate the invention. They shall not be construed, whatsoever, to limit the scope of the invention.

### EXAMPLE 1

### Cell Culture

Human embryonic kidney cells with SV40 large T-antigen (HEK 293T) were maintained in phenol red-free Dulbecco's Modified Eagle Medium (DMEM) supplemented with 10% fetal calf serum (Biochrom AG), 2 mM L-glutamine (Invitrogen/Gibco), 100 U/ml penicillin and 100 mg/ml streptomycin (Invitrogen/Gibco). Cells were cultivated at 37° C. and 5% CO2 and passaged when reaching ˜90% confluency. Before usage, the cell line was authenticated and tested tor mycoplasma contamination using the commercial Multiplex Cell Line Authentication and Mycoplasma Test services (Multiplexion).

### EXAMPLE 2

### Plasmid Construction

Constructs were generated using classical restriction enzyme cloning. Oligonucleotides were obtained from Sigma-Aldrich or Integrated DNA Technologies (IDT) and codon-optimized DMA sequences were purchased as gBlocks from IDT. Restriction enzymes were purchased from New England Biolabs and Thermo Fisher. PCR amplification of DNA fragments was performed using primers at a concentration of 0.5 μM with Phusion® High-Fidelity DNA Polymerase, by Thermo Fisher, or Q5® High-Fidelity DNA Polymerase, by New England Biolabs, according to the manufacturer's recommendations. Gels for electrophoresis were prepared with 1% agarose in 0.5× TAB. QIAquick Gel Extraction Kit (QIAGEH) was used to isolate DNA from prepared gel fragments. QIAquick Nucleotide Removal Kit or QIAquick PCR Purification Kit (both QIAGEN) were used for purification of DNA fragments without gelelectrophoresis from enzymatic digests or PCR reactions. QIAprep Spin Miniprep Kit and QIAQEN Midi Kit (both QIAGEN) were used for isolation of plasmid DNA for subsequent cloning, sequencing or transfection. Plasmids and ligation products were transformed into chemically competent E. coli TOP 10 and plated without recovery in liquid culture. Bacteria were cultivated on LB-agar plates or in LB-liquid cultures with 100 μg/ml ampicillin at 37° C. Sequences of new plasmids were validated through Sanger sequencing using the services of GATC.

### EXAMPLE 3

### Luciferase Assay

HEK 293T cells were seeded into black, clear-bottom 96-well plates (Corning) at a density of ˜12.500 cells per well Twenty-four hours after seeding, cells were transfected according to the manufacturer's instructions using Lipofectamine® 2000 and Opti-MEM® reduced serum medium (Thermo Fisher).

For the dCas9-VP64 trans-activation assays, 50 ng/well of each, a plasmid encoding dCas9-VP64_GFP (Addgene plasmid #61422, kind gift from Feng Zhang), a tet-inducible firefly luciferase reporter (Addgene plasmid #64127, kind gift from Moritoshi Sato) and a TetO-targeting guide RNA vector (Addgene plasmid #64161, kind gift from Moritoshi Sato) were co-transfected alongside 1 ng/well pRL-TK (TK-driven Renilla expression vector for normalization; Promega) as well as 1-50 ng/well of the AcrIIA4 constructs and, optionally, stuffer DMA (pcDNA3.1(−) (Invitrogen)). For the reporter cleavage assay, a plasmid encoding an H1 promoter-driven guideRNA as well as firefly luciferase and Renilla luciferase was co-transfected alongside Cas9 expression vector pSpCas9(BB)-2A-GFP (PX453; Addgene plasmid #48138, kind gift from Feng Zhang) as weft as the corresponding AcrIIA4 variant (50 ng/well each).

To assess the effects of NLS fusion to AcrIIA4, 40 ng/well of the reporter construct, 40 ng/well of plasmid encoding Cas9 and 120 ng/well of the AcrIIA4 constructs or stuffer DNA were co-transfected. To investigate the different AsLOV2 insertion sites in AcrIIA4, typically 50 ng/well of each vector encoding the luciferase cleavage reporter, Cas9 and the AcrIIA4-LOV2 hybrid were co-transfected.

Six h post-transfection, medium was replaced and the cells were illuminated with 460 nm pulsatile blue light (light intensity 2-3 W/m² as measured with a LI-COR LI-250A Light Meter, pulsatile illumination regime is indicated in figure legends) or kept in the dark under otherwise identical conditions for 32-42 h (indicated in figure legends). A custom-made LED device composed of six high-power LEDs (type CREE XP-E D5-15; LED-TECH.DE) empowered by a Switching Mode Power Supply (Manson, model: HCS-3102) served as light source. A Raspberry Pi with a custom-made python script was used for light intensity and pulsing control. Subsequently, the luciferase activity was measured using the Dual-Glo luciferase assay kit (Promega) according to the manufacturer's protocol. In brief, the cells were lysed using the provided lysis buffer and tire activity of firefly and Renilla luciferase was quantified using a GLOMAX 96 Microplate Luminometer (Promega) with automated injectors (delay time 2 s, integration time 10 s). The relative luciferase activity was calculated by dividing the firefly luciferase photon counts by the Renilla luciferase photon counts. In some eases, for reasons of comparison, the average relative luciferase activities were then normalized to the corresponding light/dark values of the reporter maximum control (cleavage assay=reporter control transactivation assay=dCas9-VP64 control) (indicated with “normalized” on the figure axis).

### EXAMPLE 4

### T7 Assay

HEK 293T cells were seeded into transparent 96-well plates (Coming). The next day, cells were transfected with equal amounts of Cas9 expression vector, CFTR guideRNA (sequence 5-GAATGGTGCCAGGCATAATCC-3′, SEQ ID NO: 29) expression vector as well as vector encoding wild-type AcrIIA4 or AcrIIA4-LOV2 hybrid (carrying AsLOV2 inserted between N64 and Y67) using Lipotectamine 2000. Sixteen It post-transfection, cells were irradiated with blue light (7 s ON, 7 s OFF, 3 W/m²) for 32 h or kept in the dark as control. Subsequently, cells were washed with PBS and lysed using the DirectPCR lysis reagent (PcqLab). A fragment spanning the edited part of the CFTR locus was PCR-amplified using Q5 polymerase and the following primers: CFTR_fw: 5′-GCACATAGAACAGCACTCGAC-3′, SEQ ID NO: 30; CFTR_re: 5′-GATCCATICACAGTAGCTTACCC-3′, SEQ ID NO: 31). The PCR reaction was run on an agarose gel and the amplicon was purified using a QIAGEN Gel Extraction Kit. Two hundred ng of PCR diluted in 19.5 μl 1× NEB Buffer 2 were then healed up to 95° C. followed by re-annealing in a thermocycler. Next, 0.5 μl of T7 Endonuclease (NEB) were added, and the reaction was incubated at 37° C. for 15 min and then stopped by adding EDTA-containing gel loading dye. Lastly, T7 reactions were analyzed on a 2% agarose gel stained with GelRed (Biotium, Inc.).

### EXAMPLE 5

### Control of AcrIIA4 by Light-Induced Disorder

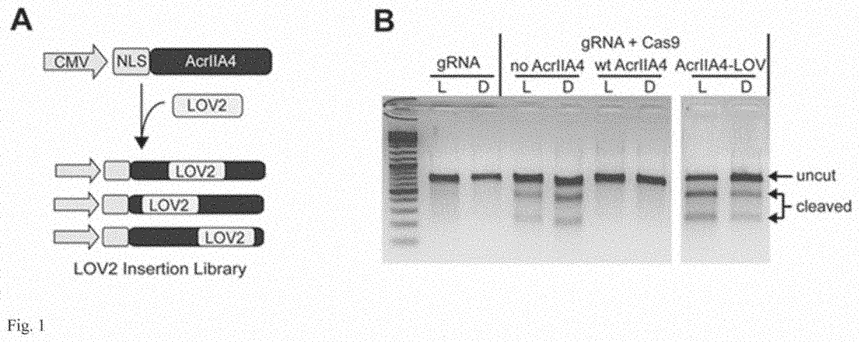

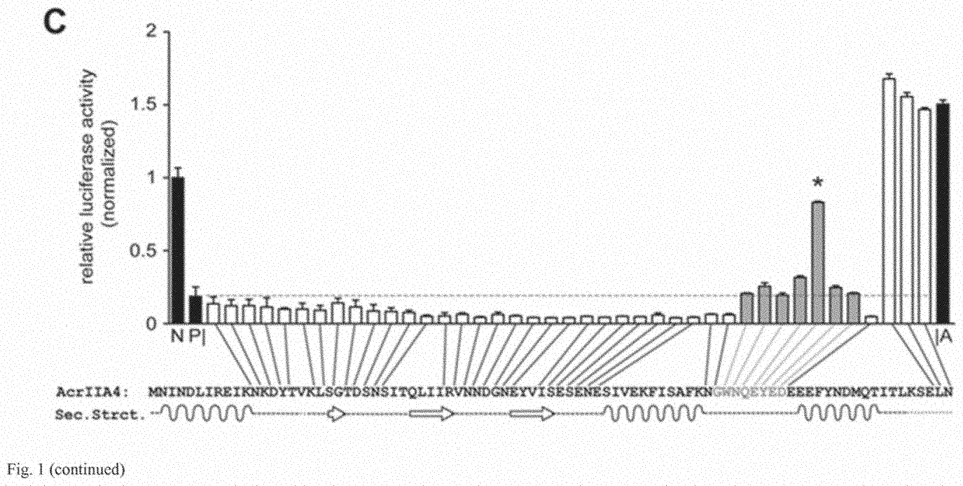

We investigated whether AcrIIA4 generally tolerates insertion of a receptor into a surface-exposed region, ideally a loop. As receptor, we chose the LOV2 blue light sensor from Avena sativa phototropin-1 (residues L404-L546 of Genbank Acc No: AAC05083.1, SEQ ID NO: 34). 41 different AcrIIA4 positions were chosen for insertion the AsLOV2 domain (As phototropin-1 residues L104-LS46) without any additional linkers (FIG. 1A and C, bottom). A comparison of insertion sites with the recently reported AcrIIA4 (secondary) structure (Dong et al, 2017) is included in (FIG. 1C, bottom).

Next, we tested the AcrIIA4-LOV2 hybrids for their ability to inhibit Cas9 catalytic activity using a luciferase reporter cleavage assay (FIG. 1C). One loop spanning from G62 to D69 tolerated A&LOV2 insertions. Cas9 inhibition was highest for the AcrIIA4 variant bearing AsLOV2 between residues E66 and Y67 (FIG. 1C; indicated with *). This is particularly surprising, as residue Y67 as well as neighboring residues D69 and H70 mediate important interactions with the PAM-binding residues of Cas9 and are thus of high importance for Cas9 inhibition by AcrIIA4 (Dong et al., 2017).

It thus appeared plausible that light-induced unfolding of the LOV2 terminal helices, in particular the AsLOV2-Jα directly preceding residues Y67/D69/E70, could disturb the Cas9 binding of AcrIIA4, thereby releasing Cas9 activity. To prove that light-induced unfolding of the LOV2 terminal helices interferes with Cas9 binding of AcrIIA4. HEK. 293T cells were co-transfected with a Cas9 construct, a vector expressing a CFTR locus-targeting guideRNA as well as a vector encoding the AcrIIA4 variant with the LOV2 domain inserted between residues E66/Y67 or wild-type AcrIIA4 as control. Note, that the ratio of Cas9:AcrIIA4 construct was 1:4.Sixteen h post transfection, cells were exposed to blue light for 32 h or kept in the dark, followed by T7 assay to measure target locus editing. As anticipated, the AcrIIA4-LOV2 hybrid inhibited Cas9 in a light-dependent manner, indicated by the increased editing of the CFTR locus in the light as compared to the dark control sample (FIG. 1B).

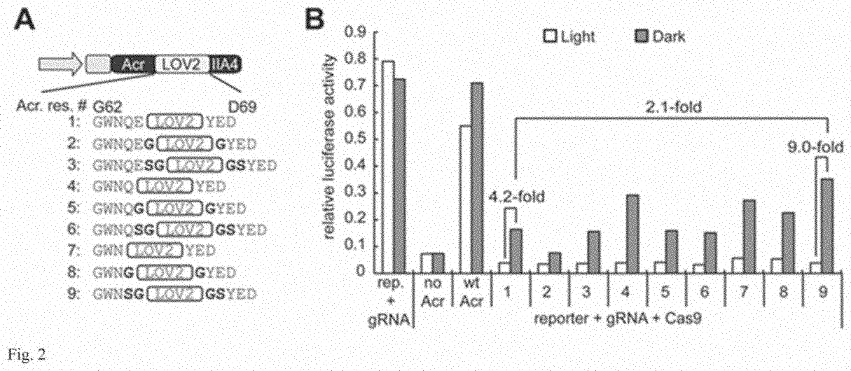

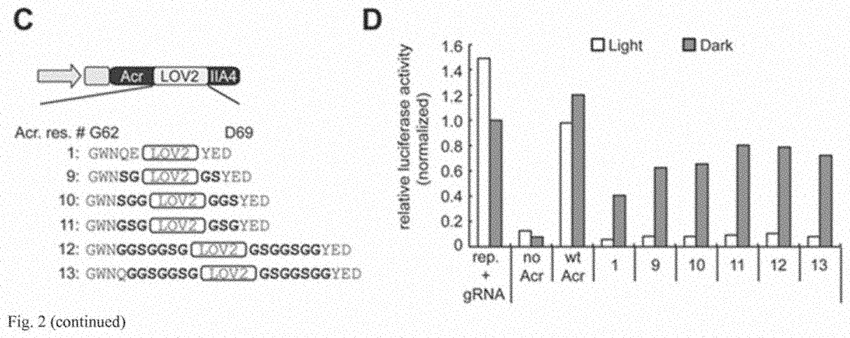

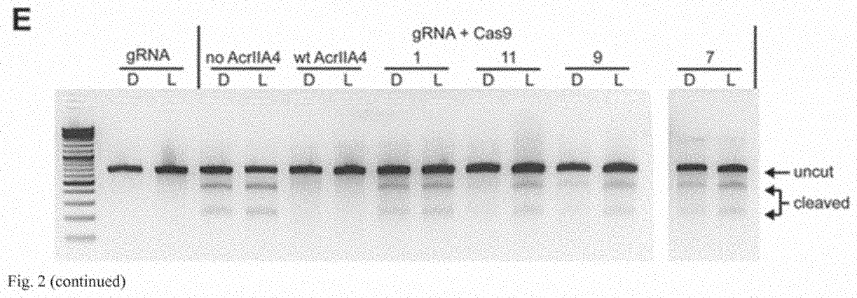

Noticeable CFTR editing was also observed in the dark sample, suggesting that the AcrIIA4-LOV2 hybrid did not fully block Cas9. Next, it was tested whether inserting short, flexible linkers at the LOV2-AcrIIA4 junction sites could improve Cas9 inhibition in the dark. Using our lead construct (LOV2 inserted between AcrIIA4 residues E66/YC7; FIG. 2A, construct #1) as scaffold, we generated a set of 8 additional AcrIIA4-LOV2 hybrids bearing short G or GS linkers at the LOV termini and optionally an E66 single or Q65/E66 double deletion on AcrIIA4 (FIG. 2A, constructs 42-9). The newly generated variants were transfected into HEK 293T cells together with a Cas9 expression vector as well as a corresponding luciferase cleavage reporter. Cells were irradiated with blue light for 42 h or kept in the dark, followed by luciferase assay (FIG. 2B). Several new variants (#4, #7, #8 and #9) outperformed the parental variant (#1) and showed a potent Cas9 inhibition in the dark as well as strong light-induced release of Cas9 catalytic activity (FIGS. 2A and B). Variant #9 (FIGS. 2A and B) showed a ˜2-fold increase in Cas9 inhibition in the dark compared to the parental construct (#1) and a strong (9-fold) increase in Cas9-mediated reporter knockdown upon irradiation.

Notably, this candidate bears the AcrIIA4 Q65/E66 double deletion and LOV2-flanking GS linkers.

Based on this knowledge, further constructs bearing either the Q65/E66 double or E66 single deletion with elongated GS linkers were designed (FIG. 2C) and evaluated for light-dependent Cas9 inhibition using a luciferase assay as before (FIG. 2D). This identified LOV-AcrIIA4 hybrids that arc even more potent at inhibiting Cas9 in the dark and strongly releasing this inhibition upon irradiation (FIG. 2D. compare construct 9 to constructs 10-13).

A subset of these newly created LOV2-AcrIIA4 hybrids (construct 7, 9, 11) was investigated for their ability to control Cas9 editing of the endogenous CPTR locus by T7 assay and compared to the parent variant (construct #1). In an assay using a ratio of transfected Cas9:AcrIIA4 construct of 1:1, the parent LOV2-AcrIIA4 hybrid gave no significant Cas9 inhibition in the dark (FIG. 2E, construct 1). In contrast, the new variants showed moderate (construct 7) to highly potent (constructs 9 and 11) Cas9 inhibition in the dark and a strong release of Cas9 activity upon activation (FIG. 2E). indicating successful light control of Cas9 editing of an endogenous locus using our engineered LOV2-AcrIIA4 hybrids.

### Example 6

### Rapamycin Control of Cas9 Activity

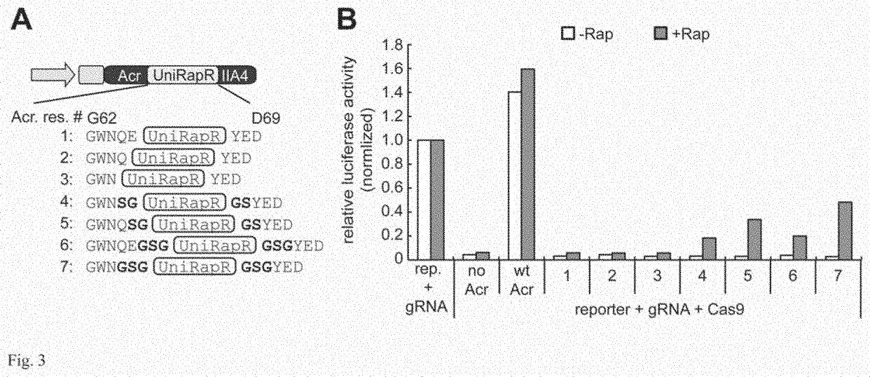

To evaluate whether chemical triggers could be used for systemic Cas9 control, e.g. in animals, we replaced the light input by a clinically approved drug. To this end, we employed the UniRapR receptor, a previously reported FRB-iFKBP fusion (Dagliyan et al., 2013), whose conformation is stabilized upon rapamycin binding. Using the LOV2-AcrIIA4 hybrids as blueprint (FIG. 2), we inserted the UniRapR domain between AcrIIA4 residues N64 and Y67 (FIG. 3A). We again introduced optional GS-linkers at the AcrllA4-UniRapR interfaces as well as Q65/E66 deletions on AcrIIA4 (FIG. 3A), both of which has been beneficial in the context of the LOV2-AcrIIA4 hybrids engineered before. The generated constructs were then investigated for their ability to inhibit Cas9 mediated luciferase reporter cleavage in HEK 293T cells in the presence or absence of 10 μM rapamycin (FIG. 3B). Remarkably, in particular engineered UniRapR-AcrIIA4 hybrids comprising linkers showed a prominent regulation of Cas9 catalytic activity upon rapamycin treatment (FIG. 3B, constructs #4-7). Thus, rapamycin treatment activates AcrIIA4 which in turn results in Cas9 inhibition, while AcrIIA4 is disordered (fully inactive) in the absence of the trigger (FIG. 3B).

Of note, the lead candidate obtained from this initial, small UniRapR-AcrIIA4 hybrid screen (construct 7, FIGS. 3A and B) not only shares the GSG-thinking linkers, but also the beneficial Q65/E66 double deletion with the lead candidate of our LOV2-AcrIIA4 screen (compare construct 11 m FIG. 2C-E and construct 7 in FIG. 3). It can thus be concluded that the AcrIIA4 engineering hotspot identified in this work can be targeted by different receptors to control Cas9 inhibition with diverse triggers.

### Example 7

### N-terminal photoreceptor fusion also enables AcrIIA4 light control

An allosteric AcrIIA4 light-switch was also created by connecting the rigid C-terminal LOV2 Jα helix and the N-terminal AcrIIA4 helix. To this end, we generated different LOV2-AcrIIA4 fusions, in which the LOV2-Jα-AcrIIA4 interface was optionally modified as follows. We either (i) inserted previously described mutations into the Jα helix (FIG. 4A; #41, #51, #53-41, #69-41, #71-51), that could improve helix docking and photoswitching (Niopek et al, 2016), (ii) introduced short, N-terminal AcrIIA4 deletions (FIG. 4A; #53-41) or (iii) inserted short linkers between the C-terminal LOV2 and the N-terminal AcrIIA4 helices (FIG. 4A: #69-41, #71-51). The resulting LOV2-AcrIIA4 hybrids were then tested using either a dCas9-VP64 luciferase reporter trans-activation assay (FIG. 4B) or a Cas9 luciferase reporter cleavage assay (FIG. 4C). As hypothesized, the constructs showed noticeable light-dependent dCas9-VP64 and Cas9 inhibition (FIGS. 4B and C), although the dynamic range of regulation was modest (2- to 3-fold change in reporter activity upon irradiation).

### Example 8

### Light-Induced AcrIIA4 Cytoplasmic Sequestration Releases Cas9 Activity

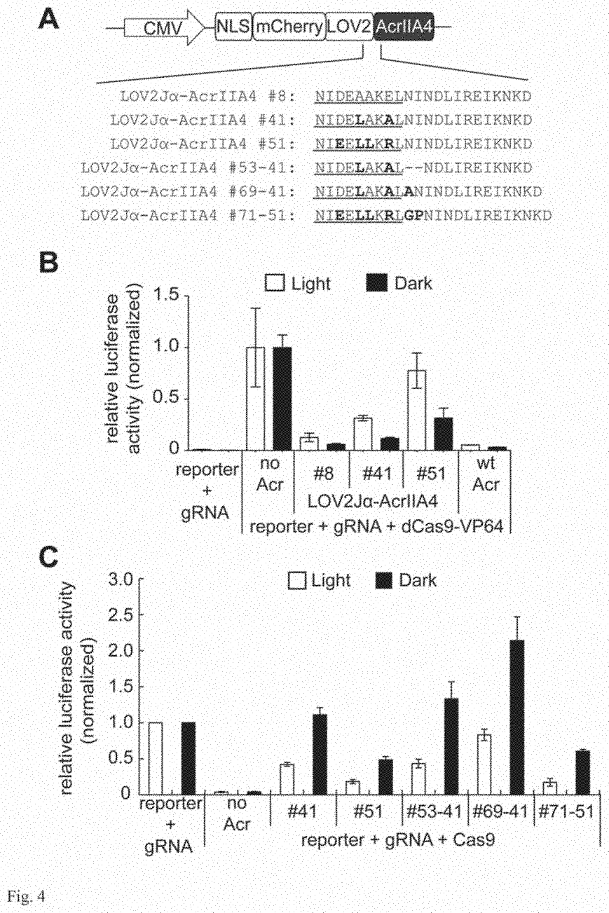

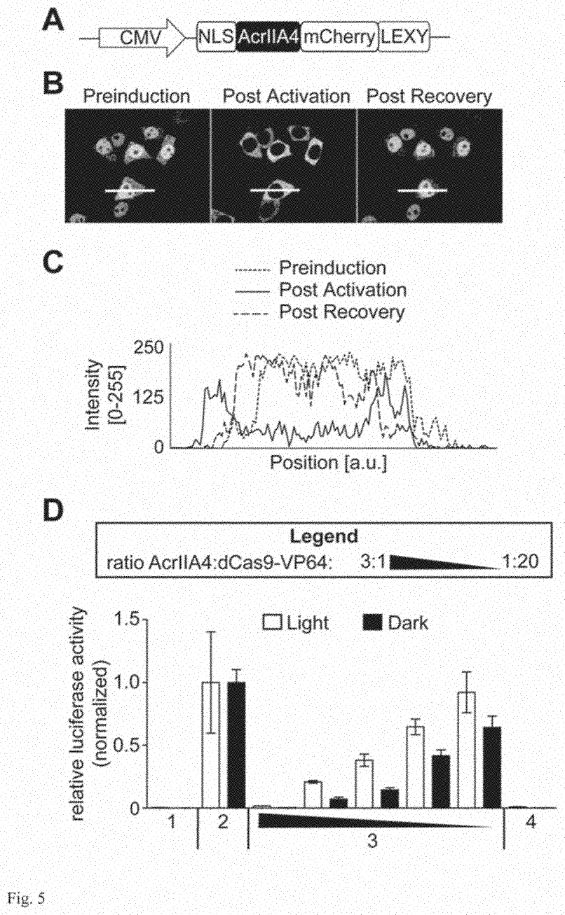

To test whether sequestering AcrIIA4 away from the nucleus could be a possible mode for allosteric Acr control we employed a light-inducible nuclear export system (LEXY) previously reported (Niopek et al., 2016), which mediates the nuclear export of proteins fused to the LEXY domain (an AsLOV2-Nuclear Export Signal-Hybrid) in response to blue light. We generated a CMV promoter-driven construct expressing an NLS-tagged AcrIIA4 fused to mCherry (for visualization purposes) and LEXY (FIG. 5A), and transfected it into HEK 293T cells. Upon blue-light irradiation, we observed a fast and strong decrease in nuclear mCherry fluorescence, indicating successful sequestration of the AcrIIA4 fusion proteins into the cytosol (FIGS. 5B and C). The nuclear mChcrry fluorescence fully recovered within 20 min when blue-light irradiation was stopped, showing that AcrIIA4 nuclear export was reversible (FIGS. 5B and Q.

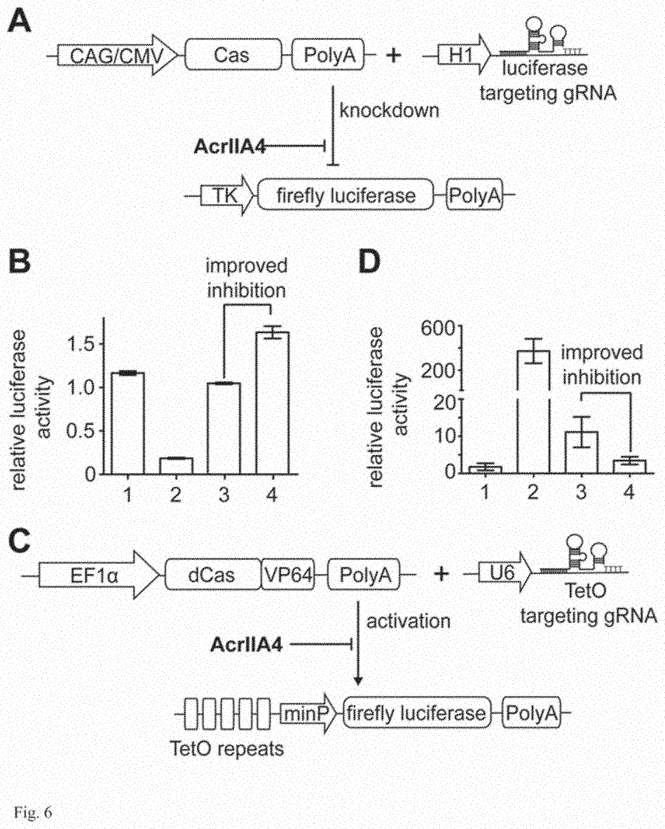

Finally, we tested whether cytosolic AcrIIA4 sequestration had an impact on Cas9 activity. To this end, we co-transfected HEK 293T cells with different amounts of the NLS-AcrIIA4-mCherry-LEXY fusion-encoding vector, dCas9-VP64 and a corresponding luciferase reporter (see FIG. 6C). Cells were then exposed to blue light for 48 h or kept in the dark, followed by luciferase assay.

As expected, nuclear export of AcrIIA4 caused a noticeable increase in dCas9-VP64-mediated luciferase reporter induction, indicating successful light control of AcrIIA4 (FIG. 5D). As moreover expected, the efficiency of dCas9-VP64 inhibition and release upon irradiation was dependent on the used NLS-AcrIIA4-mCherry-LE XY dose (FIG. 5C). Of note, the dynamic range of regulation was modest and concentration-dependent, indicating that cytosolic sequestration was not 100% efficient or that AcrIIA4-bound dCas9-VP64 was partially co-exported.

### Example 9

### AcrIIA4 Nuclear Targeting Improves Cas9 Inhibition

We aimed at independently verifying the results by Rauch et al. (2017) suggesting that AcrIIA4 efficiently inhibits target DNA binding of the catalytically active SpyCas9 as well as the catalytically impaired dCas9 mutant. Therefore, we co-transfected HEK 293T cells with vectors expressing Cas9, a firefly luciferase reporter also encoding a firefly luciferase targeting guideRNA as well as two different AcrIIA4 vectors differing by the presence or absence of an additional N-terminal SV40 NLS (nuclear localization signal). In the absence of AcrIIA4, Cas9 caused a prominent firefly luciferase knockdown, which was strongly reduced upon co-expression of AcrIIA4 (FIG. 6B). Remarkably, the AcrIIA4 construct bearing the additional NLS resulted in a more potent Cas9 inhibition, as reflected by a full recovery of the luciferase reporter signal (FIG. 6B). This boost in AcrIIA4 activity due to nuclear targeting could be further confirmed using a cMyc^{PIA}-NLS-tagged AcrIIA4 variant in combination with a dCas9-VP64 trans-activator and corresponding reporter test system (FIGS. 2C and D).

These experiments verify the reported, high potency of AcrIIA4 at inhibiting SpyCas9 or dCas9. They further show that targeting AcrIIA4 to the nucleus improves Cas9 inhibition, likely by increasing the AcrIIA4:Cas9 ratio in the relevant cellular compartment. Therefore, we included an N-terminal NLS into all AcrIIA4 constructs used in the Examples above.

### Example 10

### Materials and Methods for Examples 11 to 13

### Computational Design of Improved Acr-LOV Mutants

Interface design was performed for the interface residues in AcrIIA4 using the RosettaScripts application (Fleishman et al (2011). In silico saturation mutagenesis was performed for residues in close spatial proximity (residue set 1:16, 18, 33 and set 2:19, 28, 45). Designs with interaction energies (ddGs) within the same range (+2.5 rosetta energy units) or lower than that of the wild-type complex were manually inspected and the best mutations were selected for experimental characterization. Table 1 presents the metrics of the mutants experimentally characterized.

TABLE 1
CASANOVA mutants selected for experimental characterization.
ddG indicates the predicted change in free energy upon binding to
the Cas9/gRNA complex. The dHbond_gain_overall shows the number
of additionally formed buried hydrogen bonds of the designs
compared to the wild-type (baseline).

| Construct | Rosetta score | ddG | dHbond_gain_overall |
|---|---|---|---|
| baseline | −1434.482 | −124.294 | 0 |
| T16Y | −1412.166 | −124.173 | 1 |
| T16F | −1432.37 | −125.793 | 0 |
| K18Q | −1435.027 | −125.65 | 3 |
| T22H | −1432.85 | −125.192 | 0 |
| T28E | −1436.272 | −124.224 | 0 |
| T28N | −1433.502 | −125.348 | 1 |
| T28Q | −1440.055 | −125.657 | 0 |
| E45K | −1438.617 | −124.158 | 1 |
| S46D | −1396.953 | −122.129 | 1 |
| N64K | −1435.808 | −125.231 | 0 |
| N64R | −1409.696 | −124.297 | 1 |

General Methods and Cloning

Plasmids were created using classical restriction enzyme cloning, Golden-gate cloning (Chen et al. (2013)) or Gibson assembly (New England Biolabs). Oligonucleotides were obtained from IDT or Sigma Aldrich. Synthetic, double-stranded DNA fragments were obtained from IDT. The CMV promoter-driven SpyCas9 expression vector was obtained by PCR-amplifying the SpyCas9 gene from vector pSpCas9(BB)-2A-GFP (kind git) from Feng Zhang (Addgene plasmid #48138)) followed by ligation into pcDNA3.1^{(.)} (ThermoFisher) via XhoI/HindIII. AAV vectors encoding SpyCas9 or a U6 promoter-driven, improved gRNA scaffold (F+E Chen et al. (2013)) and RSV promoter-driven GFP (Senis et al. (2014)) were employed for gRNA expression. Annealed oligonucleotides corresponding to the target site sequence were cloned into the gRNA AAV vector via BbsI using Golden-gate cloning. The luciferase reporter for measuring SpyCas9 activity (luciferase cleavage reporter) was developed by cloning an H1-driven expression cassette encoding a firefly luciferase-targeting gRNA into pAAVpsi2Borner et al. (2013). The resulting vector co-encodes an SV40 promoter-driven Renilla luciferase gene and a TK promotor-driven Firefly luciferase gene. The AcrIIA4 coding sequence was obtained as human codon-optimized, synthetic DNA fragment from IDT and cloned into pcDNA3.1^{(.)} via NheI/NotI. Acr-LOV hybrids were created by linearizing the Acr-encoding vector by PCR followed by insert ion of a human codon-optimized Avena saliva LOV2-encoding fragment (IDT) via blunt-end ligation or Golden-gate cloning. GS linkers were optionally appended to the LOV-encoding DNA fragment via PCR prior to ligation. Mutations were introduced into the Acr part of the Acr-LOV hybrids by site-directed mutagenesis using 5′ phosphorylated primers. Mutations were inserted into the LOV part of the Acr-LOV hybrids by PCR-amplifying the LOV2 domain with primers introducing the mutations into the N- and C-terminal helix and cloning the altered LOV fragment back into a PCR-linearized, patent Acr-LOV hybrid vector using Golden-gate cloning. Note that wild-type Acr as well as all Acr-LOV hybrids bear an N-terminal SV40 nuclear localization signal, which we added to target the Cas9 inhibitor to the nucleus. The xCas9 cDNA was created by Gibson assembly on basis of the reported SpyCas9 mutations (Hu et al. (2018)) using synthetic, double-stranded DNA fragments cloned into pcDNA3.1^{(.)}. The dCas9-p300 construct was a kind gift from Charles Gersbach (Addgene plasmid #61357). pEJS477-pHAGH-TO-SpydCas9_3XmCherry-sgRNA/Telomere-All-in-one was a gift from Erik Sontheimer (Addgene plasmid #85717). Based on this vector, constructs co-expressing dCas9_3XmCherry and CASANOVA or wild-type AcrIIA4 via a P2A peptide were created by cloning a P2A-CASANOVA or P2A-AcrIIA4 cDNA (IDT) behind the SpyCas9-3XmCherry coding sequence.

In all cloning procedures, PCRs were performed using Q5 Hot Start High-Fidelity DNA Polymerase (New England Biolabs) or Phusion Flash High-Fidelity polymerase (ThermoFisher). Agarose gel electrophoresis was used to analyze PCR products. Bands of the expected size were cut out and DNA extracted using a QIAquick Gel Extraction Kit (Qiagen). Ligations were performed using T4 DNA ligase (New England Biolabs) and optionally heat-inactivated at 70° C. for 45 min before transformation. Chemically-competent Top 10 cells (ThermoFisher) were used for DNA vector amplification. Plasmid DNA was purified using the QIAamp DNA Mini, Plasmid Plus Midi or Plasmid Maxi Kit (all from Qiagen).

Cell Culture, Transient Transfection and AAV Lysate Production

Cells lines w ere cultured at 5% CO₂ and 37° C. in a humidified incubator and passaged when reaching 70 to 90% confluency (every two to four days). HEK 293T (human embryonic kidney) and U2OS (human osteosarcoma; kindly provided by Karsten Rippe, German Cancer Research Center (DKFZ), Heidelberg) were maintained in phenol red-free Dulbecco's Modified Eagle Medium (DMEM; ThermoFisher/GIBCO) supplemented with 10% (v/v) fetal calf scrum (Biochrom AG), 2 mM L-glutamine and 100 U per ml penicillin/100 μg per ml streptomycin (both ThermoFisher/GIBCO). The U2OS medium was additionally supplemented with 1 mM sodium pyruvate (GIBCO). Cell lines were authenticated and tested for mycoplasma contamination prior to use via a commercial service (Multiplexion). Transient transfections were performed with JetPrime (Polyplus transfection) or Turbofect (ThermoFisher) according to the manufacturer's protocols. Details are listed in the corresponding experimental sections below. For production of AAV-containing cell lysates, low-passage HEK 293T cells were seeded into 6-well plates (CytoOne) at a density of 350,000 cells per well. The following day, cells were triple-transfected with (i) the AAV vector plasmid, (ii) an AAV helper plasmid carrying AAV serotype 2 rep and cap genes, and (iii) an adenoviral plasmid providing helper functions for AAV production, using 1.33 μg of each construct and 8 μl of Turbofect reagent per well. The AAV vector plasmid encoded either (1) Cas9 driven from an engineered, short CMV promoter (Senis et al. (2014)), (2) a U6 promoter-driven gRNA (Senis et al. (2014)) (based on the improved F+E scaffold: Chen et al. (2013)) and a RSV promoter-driven GFP marker, or (3) a CMV promoter-driven CASANOVA variant. Seventy-two hours post-transfection, cells were collected in 300 μl PBS and subsequently subjected to five freeze-thaw cycles by alternating between snap freezing in liquid nitrogen and 37° C. Finally, the cell debris was removed by centrifugation at ˜18,000 g and the AAV-containing supernatant was stored at −20° C. until use.

Blue Light Setup

For blue light illumination of samples in the cell culture incubator, a custom-made blue light setup comprising six blue light, high-power LEDs (type OREL XP-E D5-15; emission peak ˜460 nm; emission angle ˜130°; LED-TECH.DE) empowered by a Switching Mode Power Supply (Manson, model: HCS-3102) was used. A Raspberry Pi running a custom Python script was used to control the power supply. Samples were irradiated from below, i.e., through the transparent bottom of the culture dishes or well plates by positioning them on an acrylic glass table installed in the incubator, with the LEDs being located underneath the table. A pulsatile illumination regime (5 s ON, 10 s OFF) was used for sample irradiation. Light intensity was ˜3 W per m² as measured with a LI-COR LI-250A light meter, unless indicated otherwise below.

Luciferase Reporter Assays

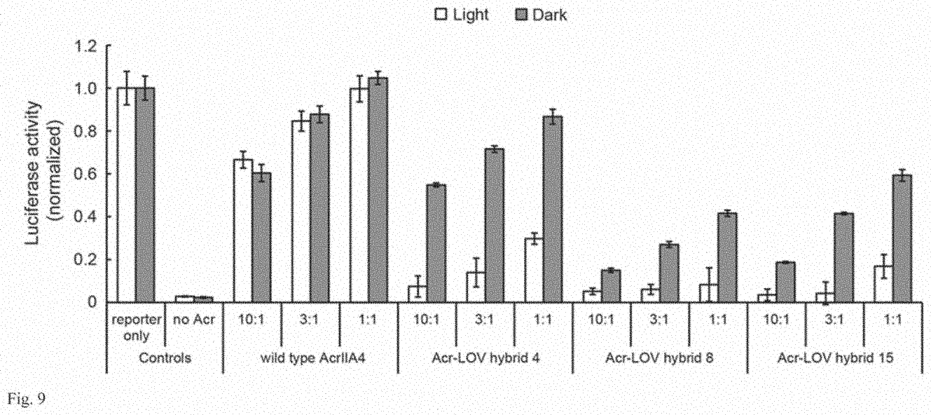

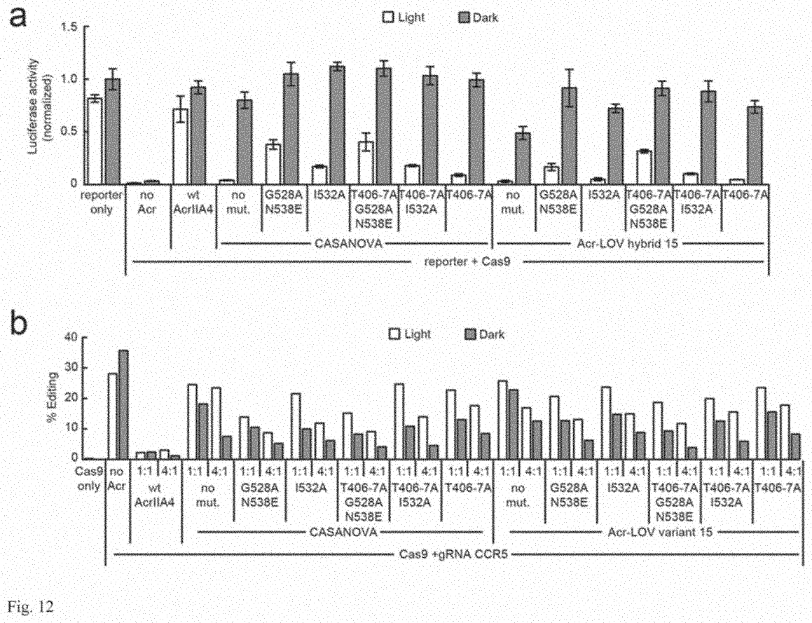

HEK 293T were seeded into black, clear-bottom 96-well plates (Corning) at a density of ˜12,500 cells per well. The following day, cells were co-transfected with 33 ng of a Cas9 or xCas9 expression vector, 33 ng of a construct co-expressing Firefly and Renilla luciferase as well as an H1 promoter-driven gRNA targeting the Firefly luciferase cDNA, and, in most cases, 33 ng of the CMV promoter-driven Acr-LOV hybrid using 0.2 μl JetPrime (amounts are per well). For the titration experiment in FIG. 9, either 3, 10 or 33 ng of Acr-LOV hybrid was co-transfected with 30, 23 or 0 ng of an irrelevant DNA to vary the vector mass ratio of Cas9 and Acr-LOV construct between 10:1 and 1:1. For the experiment shown in FIG. 12. 8.25 ng of Acr-LOV constructs and 24.75 ng of an irrelevant DNA was used. Six hours post-transfection, the medium was exchanged and cells were exposed to blue light for 48 h or kept in the dark as control For the titration experiment shown in FIG. 9. irradiation time was 30 h and light intensity ˜2.5 W per m². Subsequently, a Dual-Glo Luciferase Assay System (Promega) was applied to quantify luciferase activity. In brief, cells were harvested into the supplied lysis buffer and Firefly and Renilla luciferase activities were measured using a GLOMAX™ Discover or GLOMAX™ 96 microplate luminometer (both Promega). Integration time was 10 s, and delay time between automated substrate injection and measurement was 2 s. Firefly photon counts were normalized to Renilla photon counts and resulting values were further normalized to the positive control.

T7 Endonuclease Assay

Cells were seeded into black, clear-bottom 96-well plates (Coming) at a density of 12,500 cells per well for transfection based experiments or 3,500 cells per well for AAV transduction-based experiments. Transfections were performed with JetPrime using 0.3 μl of JetPrime reagent and 200 ng total DNA per well comprising either 33 ng of each, the gRNA, Cas9 and CASANOVA expression vectors as well as 100 ng of an irrelevant DNA (1:1 ratio Cas9:CASANOVA) or 33 ng gRNA, 33 ng Cas9 and 133 ng CASANOVA expression vector (1:4 ratio Cas9:CASANOVA). For AAV-based experiments, cells were co-transduced with 7 μl of each, the Cas9, gRNA and CASANOVA AAV lysates on two subsequent days when targeting the CCR5 locus. For CFTR and EMX1, 33 μl of each AAV lysate was used. Following transfection or transduction, cells were irradiated with blue light for 70 h or kept in the dark as control. Cells were washed with PBS and harvested in DirectPCR Lysis Reagent (Peqlab) supplemented with Proteinase K (Sigma). The genomic CRISPR/Cas9 target locus was PCR-amplified with primers flanking the target site using Q5 Hot Stan High-Fidelity DNA Polymerase (New England Bio labs).

Quantitative RT-PCR

HER 293T cells were seeded into transparent 6-well plates (CytoOne) at 250,000 cells per well. The next day, cells were co-transfected with (i) 750 ng IL1RN gRNA construct mix (Hilton et al. (2015) (187.5 ng per vector), (ii) 500 ng of a construct encoding dCas9-p300-P2A-CASANOVA (or an irrelevant DNA as control), and (iii) 250, 500 or 750 ng CASANOVA-encoding vector and 500, 250 or 0 ng of irrelevant stuffer DNA, respectively, using 6 μl JetPrime reagent (all amounts arc per well). The medium was replaced 4 h post-transfection and cells were irradiated with blue light pulses for 44 h or kept in the dark as control, before lysing cells using the QIAzol Lysis Reagent (Qiagen) according to the manufacturer's instructions. Reverse transcription was performed with the RevertAid First Strand cDNA Synthesis Kit (Thermo Fisher) and equal amounts of input RNA for each experiment. Real-time PCR reactions were set up using 2 μl cDNA mix (25 ng per μl). 1.4 μl of each 10 μM primer, respectively, 10 μl PowerSYB® Green PCR Master Mix (Thermo Fisher) and 5.2 μl water. A StepOne Plus real-time PCR system (Applied Biosystems) was employed with the following cycling conditions: 95° C./10 min initial denaturation followed by 45 cycles of (95° C./15-s-58° C./60 s). Fold-changes in IL1RN levels were then calculated using the ΔΔCt method (Livak et al., 2001).

Telomere Labeling Experiments

U2OS cells were seeded into 4-compartment CELLview cell culture dishes (Greiner Bio-One) at a density of 30,000 cells per compartment. The next day, cells were transfected with vectors encoding (i) a CMV promoter-driven dCas9-3xRFP-P2A-CASANOVA and a U6 promoter-driven telomere-targeting gRNA, (ii) a telomere-targeting gRNA and GFP transfection marker, and (iii) a CMV promoter-driven CASANOVA in a ratio of 20:6:3 using 362.5 ng total DNA and 1.5 μl JetPrime for transfection (per compartment).

In the positive control samples, vector i was replaced by a vector encoding dCas9-3xRFP (without the P2A-CASANOVA) and a U6 promoter-driven telomere-targeting RNA, and vector iii was replaced by an irrelevant DNA. In the negative control samples, the CASANOVA in vectors i and iii was replaced by wild-type AcrIIA4. Four hours post-transfection, the medium was changed and cells were either irradiated with blue light pulses for 20 h or kept in the dark followed by fixation of samples with 4% PFA. SlowFade™ Diamond Antifade Mountant with DAPI (Invitrogen) was added and imaging was performed using the aforementioned microscopy setup and the following excitation/detection settings: 405 nm (1% laser powerV410-490 nm for DAPI, 488 nm (2% laser power)/493-578 nm for GFP, or 552 nm (1% laser power)/578-789 nm for RFP. RFP fluorescent spots (i.e., labeled telomeres) were then detected and quantified using a fully automated image analysis pipeline as follows. The ImageJ2 (beta) Integration in KNIME Version 3.5.2 (KNIME AG) was used to create an automated image processing and analysis pipeline employed for quantification of labeled telomeres. Analysts of all images was performed using the identical workflow configuration, apart from the configuration of data input and output nodes. In brief, raw image stacks (Jif files) were imported into KNIME followed by splitting the three fluorescence channels (DAPI, nuclear marker; GFP, a transfection marker co-encoded on foe gRNA vector; RFP corresponding to dCas9-3XmCherry). Nuclei were segmented based on foe DAPI signal. GFP-negative nuclear segments (i.e., negative for the telomere-targeting gRNA construct) were excluded from the analysis. Furthermore, nuclear segments with a mean RFP signal higher than 170 (as images were 8 bit, this corresponds to ⅔ of the maximum) were also excluded from the analysis, as the very high RFP background fluorescence impaired reliable spot detection. The Spot Detection node was employed to identify and segment fluorescent spots in the RFP channel. All spots lying outside of the nuclear segments were excluded and random fluorescence fluctuations were filtered out by selecting for spots with an average fluorescence at least 1.7-fold higher than the RFP background fluorescence in the corresponding nuclear segment. The workflow output comprises a CSV table listing the nuclear segments and corresponding spots detected in each image. Subsequent data visualization and statistical analysis was performed in R version 3.3.2.

### Example 11

### AcrIIA4-LOV2 Variants

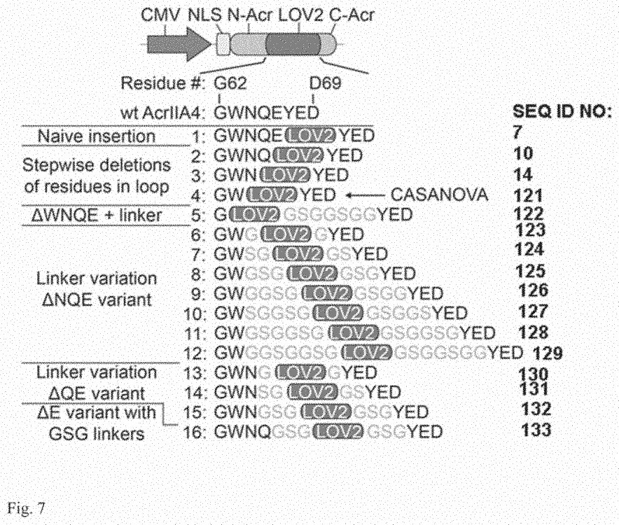

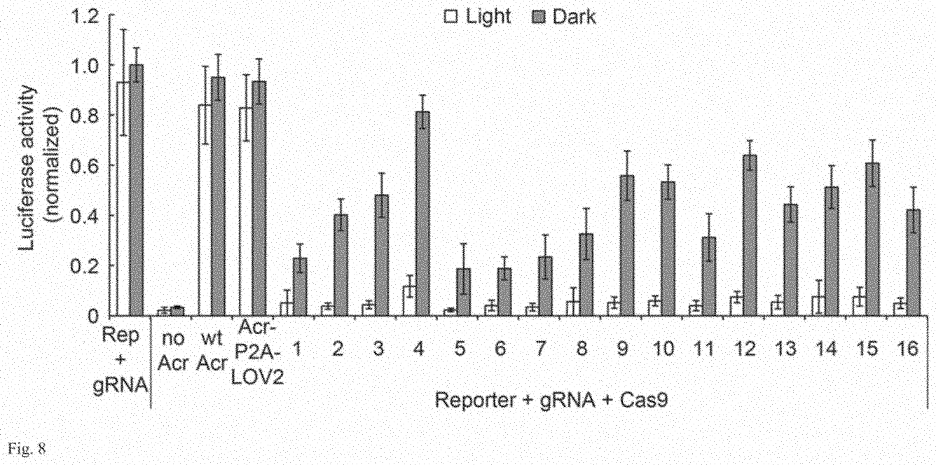

To further improve AcrIIA4-LOV2 hybrids, we aimed at embedding the LOV2 domain further into the C-terminal AcrIIA4 loop. Therefore, we stepwise deleted AcrIIA4 residues that directly precede the insertion site, but do not mediate critical contacts with Cas91 (FIG. 7, variants 2-5). Furthermore, GS-linkers of variable length were optionally included at the Acr-LOV boundaries (FIG. 7. variants 5-16). Indeed, several of the so-obtained Acr-LOV hybrids showed a potent Cas9 inhibition in the dark and almost full recovery of Cas9 activity in the light condition (FIG. 8). As expected for a competitive inhibitor, the degree of Cas9 inhibition depended on the dose of transfected Acr-LOV hybrid (FIG. 9). The most potent hybrid inhibitor (Acr-LOV variant 4) carried a three amino acid deletion (delta N64/Q65/E66) preceding the LOV domain insertion site and no GS-linkers (FIGS. 7 and 8). In the following, we will refer to Acr-LOV variant 4 or further optimized mutants thereof (see below) as CASANOVA (for CRISPR/Cas9 activity switching via a novel optogenetic variant of AcrIIA4).

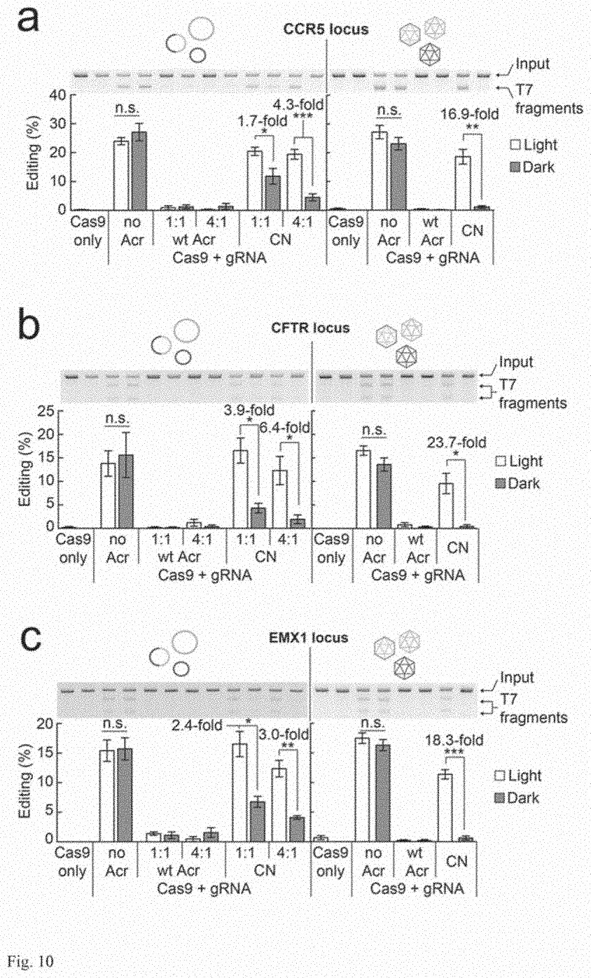

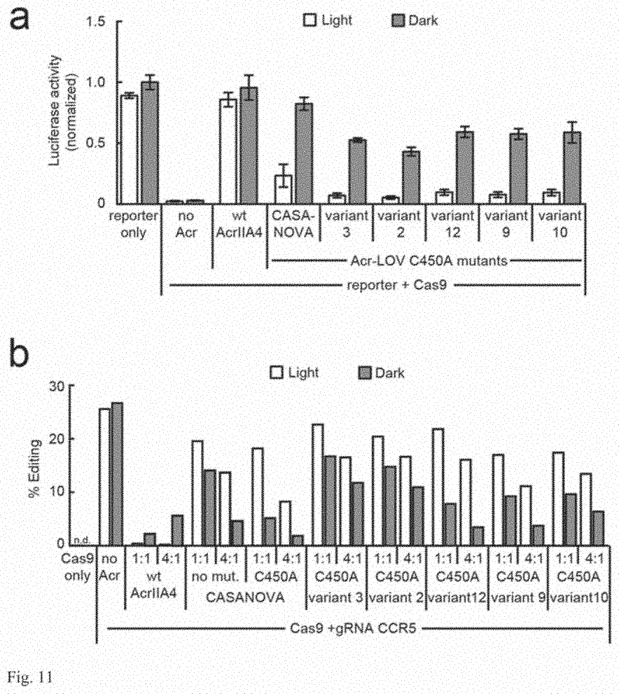

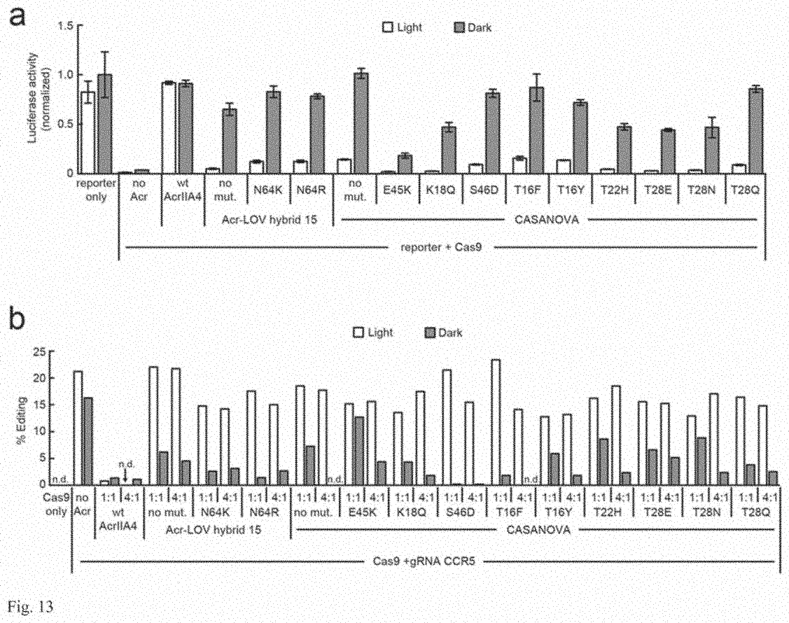

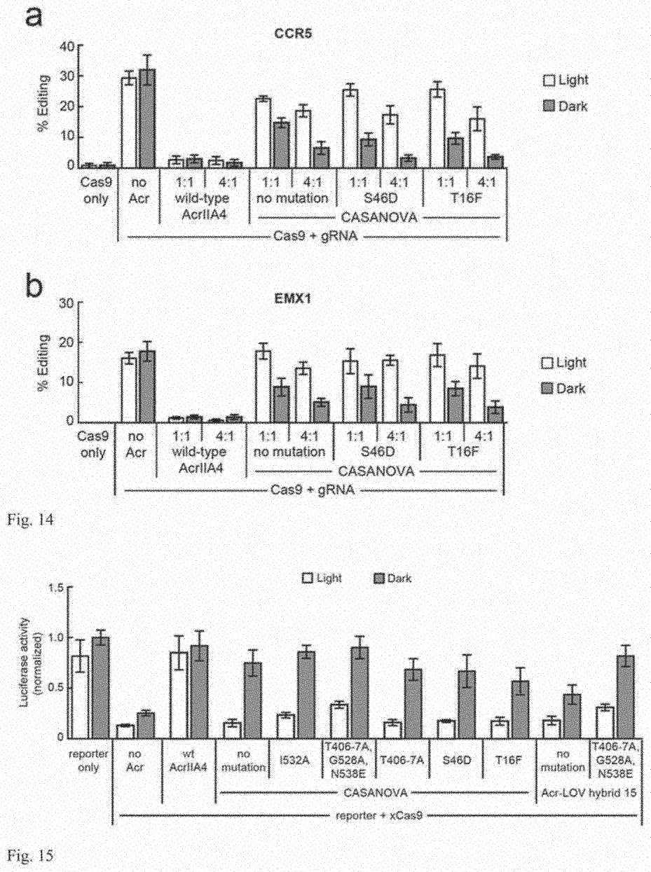

Using transient transfection or Adeno-associated virus (AAV)-mediated transduction, we co-expressed CASANOVA alongside Cas9 and gRNAs targeting the CCR5, CFTR or EMX1 locus in HEK 293T cells, indel mutations were strongly light-dependent (up to ˜24-fold regulation) for all target loci (FIG. 10). However, in the transfected samples, we observed significant background editing in the dark, suggesting Cas9 inhibition to be imperfect, at least under heterogeneous expression conditions (FIG. 10). Further, mutations known to improve docking of the terminal helices against the LOV core in the dark were introduced into the L0V2 domain of CASANOVA. As expected, Cas9 background activity was reduced in several mutants, albeit at the cost of a low er dynamic range in most eases (FIGS. 11 and 12). Performance was further tuned by introducing mutations in the AcrIIA4 part of CASANOVA. These mutations were screened computationally and selected by manual inspection and several structural metrics aiming to enhance Cas9 binding affinity (FIG. 13). Two mutants (CASAMOVAT16F and CASANOVAS46D) obtained in this manner showed enhanced Cas9 inhibition in the dark without compromising light activation noticeably (FIG. 14). CASANOVA and several of its optimized mutants also conferred strong light regulation on xCas9, a recently developed PAM-relaxed, highly specific SpyCas9 derivative2 (FIG. 15).

### Example 12

### Transcriptional Activation

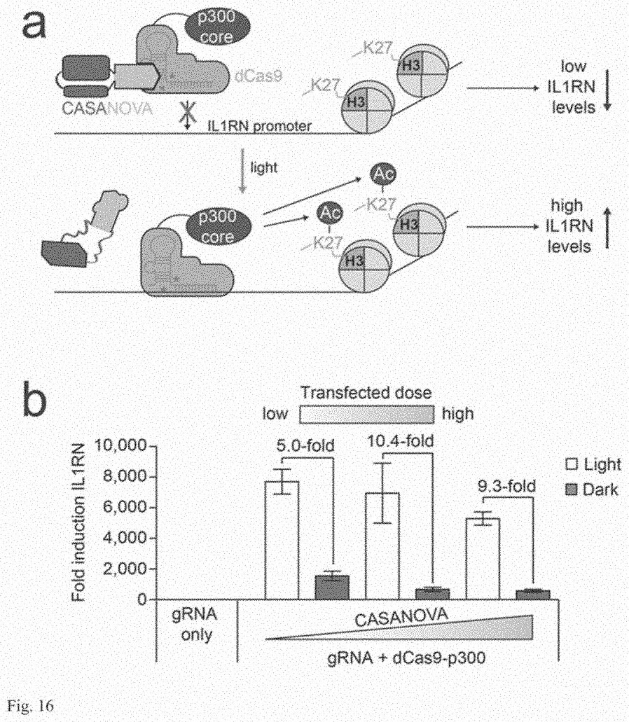

Next, we investigated whether CASANOVA would enable light-mediated regulation of d(ead)Cas9-effector fusions. To this end, we employed a previously reported dCas9 variant fused to the p300 histone acetyltransferase core domain3 and targeted the Interleukin 1 receptor antagonist (IL1RN) promoter, known to be strongly activated upon induced H3K27 acetylation, in HEK 293T cells via four different gRNAs (FIG. 16a). We titrated the transfected CASANOVA dose and incubated cells in the dark or light for 44 h before assessing IL1RN expression by quantitative RT-PCR. IL1RN transcript levels were increased up to 10-fold in the illuminated samples, indicating successful control of the dCas9-p300 epigenetic modifier (FIG. 16b).

### Example 13

### Labelling of DNA

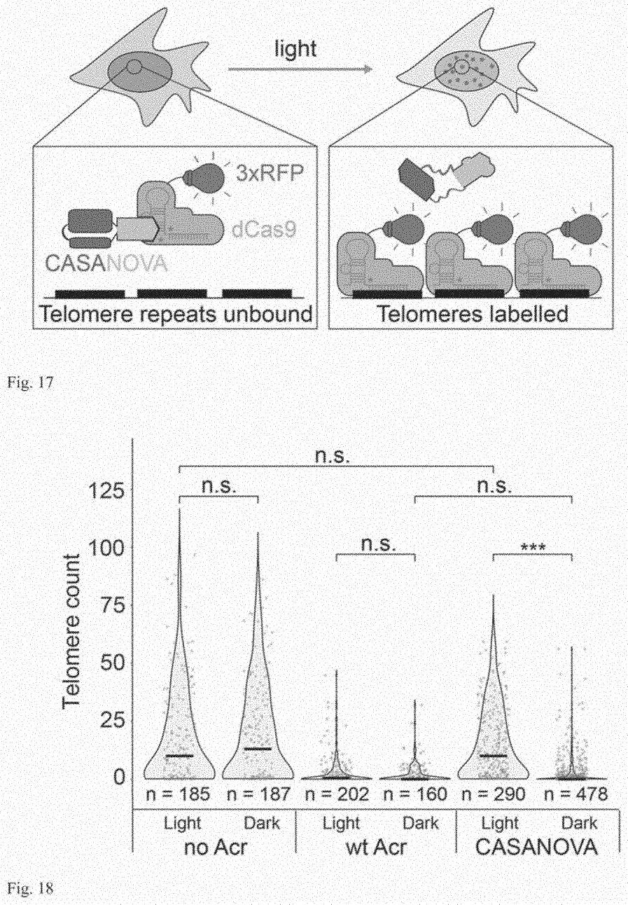

Next to conditional CRISPR/Cas9-mediated cellular perturbations, we assessed CaSANOVA's potential for studying the kinetics of Cas9 DNA targeting in living cells. To this end, we performed a CRISPR labeling experiment, in which a dCas9-3xRFP fusion, a telomere-targeting gRNA and CASANOVA were co-expressed in U2OS cells (FIG. 17). Transfected cells were incubated either in the light or dark for 20 h and samples were fixed before microscopy analysis. We also included control samples expressing wild-type AcrIIA4 instead of CASANOVA or no inhibitor at all, and analyzed telomere labeling in a fully unbiased manner using an automated image analysis workflow implemented in KNIME. The CASANOVA samples showed strong telomere labeling similar to the positive control in the light (˜80% of nuclei labeling positive; ˜40% showed more than 15 dots in the nucleus), while labeling was highly diminished and comparable to the negative control in the dark (˜60% of cells labeling negative; <5% cells showed more than 15 dots in the nucleus) (FIG. 18). The no inhibitor and wild-type AcrIIA4 controls showed light-independent, strong or weak telomere labeling, respectively (FIG. 18).

Confining CRISPR/Cas9 activity in time and space is a key prerequisite for informative genome perturbation experiments. Here, we showed that anti-CRISPR proteins can be substantially modified to render Cas9 inhibition dependent on an exogenous trigger, thereby providing a blueprint for the engineering of conditional Cas9 inhibitors. CASANOVA is not only a highly important add-on to the CRISPR toolbox, but conceptually advances our ability to confer light regulation on non-enzymatic proteins.

Non-standard literature cited:

Barrangou et al. (2007), Science 315, 1709-1712.

Bhaya et al. (2011), Annu Rev Genet 45, 273-297.

Borner et al. (2013), Nucleic Acids Res 41, e199.

Brinkman (2014), Nucleic Acids Res 42, e168.

Chen et al. (2013), Cell 155, 1479-1491.

Cong et al. (2013), Science 339, 819-823.

Daglivan et al. (2013), PNAS 110: 6800

Dai et al. (2016), Mol Ther Nucleic Acids 5, e349.

de Solis et al. (2016), Front Mol Neurosci 9, 70.

Dong et al. (2017), Nature 546, 436-439.

Fleishman et al. (2011), PloS one 6, e20161.

Engler et al. (2008), PloS one 3, e3647.

Hilton et al. (2015), Nat Biotechnol 33, 510-517.

Hu, et al. (2018) Nature 556, 57-63.

Hynes et al. (2017), Nat Microbiol 2: 1374.

Jain et al (2016), Angew Chem Int Ed Engl 55, 12440-12444.

Jinek et al. (2012), Science 337, 816-821.

Jinek et al. (2013), Elife 2, e00471.

Livak et al. (2001), Methods 25, 402-408.

Maji, et al. (2017), Nat Chem Biol 13, 9-11.

Mali et al. (2013), Science 339, 823-826.

Nihongaki et al. (2015a). Nat Biotechnol 33, 755-760.

Nihongaki et al. (2015b). Chemistry & Biology 22, 169-174.

Niopek et al. (2016), Nat common 7, 10624.

Oakes et al. (2016), Nat Biotechnol 34, 646-651.

Pawluk et al. (2016), Cell 167: 1829

Platt et al. (2014), Cell 159, 440-455.

Polstein and Gersbach (2015), Nat Chem Biol 11, 198-200.

Rauch et al. (2017), Cell 168, 150.

Schmidt and Grimm (2015), Biotechnol J 10, 258-272.

Senis et al. (2014), Biotechnol J 9, 1402-1412.

Terns and Terns (2011), Curr Opin Microbiol 14, 321-327.

Wang et al. (2013), Cell 153, 910-918.

Wiedenheft et al. (2012), Nature 482, 331-338.

Xue et al. (2016), Gene Ther 23, 557-559.

Yin et al. (2014). Nat Biotcchnol 32, 551-553.

Zetsche et al. (2015), Nat Biotechnol 33, 139-142.

1. A polynucleotide encoding a fusion polypeptide comprising an anti-CRISPR (Acr) polypeptide, wherein said fusion polypeptide further comprises a receptor domain changing conformation upon reception of a stimulus.

2. The polynucleotide of claim 1, wherein said stimulus is light, preferably blue light, or wherein said stimulus is a chemical compound, preferably is rapamycin.

3. The polynucleotide of claim 1, wherein said receptor domain is inserted into the Acr at an insertion site corresponding to one of amino acids 62 to 69 of the AcrIIA4 polypeptide (SEQ ID NO:1) and/or is fused to one of the terminal amino acids of the Acr polypeptide.

4. The polynucleotide of claim 1, wherein said receptor domain is selected from a light-oxygen-or-voltage (LOV) domain, a rapamycin-binding domain, a phytochrome (Phy) domain, a cryptochrome (Cry) domain, a steroid receptor domain, and tetracycline binding domain, preferably is a LOV domain.

5. The polynucleotide of claim 1, to wherein said fusion polypeptide comprises an amino acid sequence at least 70% identical to an amino acid sequence selected from SEQ ID NOs: 78 to 114, preferably to an amino acid sequence selected from SEQ ID NOs: 88 to 107.

6. (canceled)

7. A bipartite anti-CRISPR (Acr) polypeptide comprising a first partial Acr polypeptide comprising amino acids corresponding to amino acids 10 to 62 of SEQ ID NO: 1, and a second partial Acr polypeptide comprising amino acids corresponding to amino acids 67 to 77 of SEQ ID NO: 1.

8. The bipartite Acr polypeptide of claim 7, wherein said first and second partial Acr polypeptide are comprised in the same fusion polypeptide; or wherein said first and second partial Acr polypeptide are separately fused to the components of a receptor/ligand pair.

9. A bipartite anti-CRISPR (Acr) polypeptide comprising a first partial Acr polypeptide comprising amino acids corresponding to amino acids 10 to 62 of SEQ ID NO: 1, and a second partial Acr polypeptide comprising amino acids corresponding to amino acids 67 to 77 of SEQ ID NO: 1, wherein said bipartite Acr polypeptide is encoded by a polynucleotide according to claim 1.

10. (canceled)

11. (canceled)

12. (canceled)

13. A method of providing a host cell comprising a stimulus-modulatable activity of a CRISPR-associated (Cas) nuclease comprising
a) introducing into said host cell a Cas nuclease;
b) introducing into said host cell a fusion polypeptide comprising an Acr polypeptide and a receptor domain according to claim 9;
c) thereby, providing a host cell comprising a stimulus-modulatable activity of a Cas nuclease.

14. (canceled)

15. (canceled)

16. A method for treating genetic disease, neurodegenerative disease, cancer, and/or infectious disease in a subject suffering therefrom, said method comprising
a) contacting a host cell of said subject with a Cas nuclease and with a fusion polypeptide comprising an anti-CRISPR (Acr) polypeptide and a receptor domain according to claim 7;
b) optionally, providing a stimulus causing the receptor domain to change conformation; and
c) thereby, treating genetic disease, neurodegenerative disease, cancer, and/or infectious disease.

17. The method of claim 16, wherein said method comprises contacting at least a fraction of cells of said subject with said stimulus causing the receptor domain to change conformation.

18. The method of claim 16, wherein said method further comprises contacting said host cell with at least one gRNA.

19. The method of claim 18, wherein contacting a host cell with a gRNA is contacting said host cell with a polynucleotide comprising an expressible gene encoding said gRNA.

20. The method of claim 16, wherein contacting a host cell with a Cas nuclease is contacting said host cell with a polynucleotide comprising an expressible gene encoding said Cas nuclease.

21. The method of claim 16, wherein contacting a host cell with a fusion polypeptide comprising an Acr polypeptide and a receptor domain is contacting said host cell with a polynucleotide comprising an expressible gene encoding said fusion polypeptide comprising an Acr polypeptide and a receptor domain.
